(19) United States
(12) Patent Application Publication
(10) Pub. No.: US 2008/0269170 A1
(43) Pub. Date: Oct. 30, 2008

(54) Novel 2,4-Dianilinopyrimidine Derivatives, the Preparation Thereof, Their Use as Medicaments, Pharmaceutical Compositions and, in Particular, as IKK Inhibitors

(75) Inventors: Michael Bosch, Marsillargues (FR); Monsif Bouaboula, Juvignac (FR); Pierre Casellas, Montpellier (FR); Samir Jegham, Montferrier-Sur-Lez (FR); Serge Mignani, Chatenay-Malabry (FR); Jean-Flaubert Nguefack, Lattes (FR); Jacob-Alsboek Olsen, Francfort (DE); Bernard Tonnerre, Vailhauques (FR); Jean Wagnon, Montpellier (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 11/971,389

(22) Filed: Jan. 9, 2008

Related U.S. Application Data
(63) Continuation of application No. PCT/FR2006/001619, filed on Jul. 6, 2006.

(30) Foreign Application Priority Data
Jul. 11, 2005 (FR) 0507370
Nov. 25, 2005 (FR) 0511950

(51) Int. Cl. C07D 403/02; C07D 401/02; A61K 31/397; A61K 31/506; A61K 31/505; A61P 3/10; A61P 37/00; A61P 35/00

(52) U.S. Cl. 514/86; 544/297; 514/275; 514/210.2

(57) ABSTRACT

The disclosure relates to compounds of formula (I):

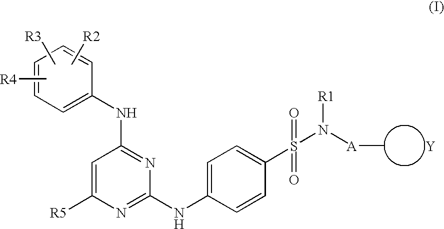

wherein R1-R5, A and Y are as defined in the disclosure, to compositions comprising said compounds, and to processes for making and methods of using the same.

The present invention relates to novel 2,4-dianilino-pyrimidine derivatives, the process for their preparation, the novel intermediates obtained, their application as medicaments, the pharmaceutical compositions containing them and the novel use of such 2,4-dianilinopyrimidine derivatives.

Patent WO 2001/64654 A1 mentions 2,4-di(hetero)aryl-pyrimidines substituted at the 5-position and which are inhibitors of the kinases CDK2 and FAK, likewise other aminopyrimidines which are inhibitors of serine-threonine kinases and of CDK are presented in WO 2003/030909 A1. Patent WO 2004/046118 A2 describes 2,4-diphenylamino-pyrimidine derivatives as cell proliferation inhibitors.

A series of 5-cyano-2-aminopyrimidines are presented as inhibitors of the kinases KDR and FGFR, in WO 2000/78731 A1, other pyrimidines as inhibitors of FAK and IGFR in WO 2004/080980 A1 and also of ZAP-70, FAK and/or Syk tyrosine kinase in WO 2003/078404 A1, and polokinases PLK in WO 2004/074244 A2, as cytostatic agents.

Likewise, other patents describe reverse transcriptase inhibiting pyrimidines for the treatment of HIV-related infections (WO 2001/85700 A2; WO 2001/85699 A2; WO 2000/27825 A1 and WO 2003/094920 A1).

The subject of the present invention is thus novel 2,4-dianilinopyrimidine derivatives having inhibitory effects on protein kinases.

The products of the present invention may thus in particular be used for the prevention or treatment of conditions capable of being modulated by the inhibition of the activity of protein kinases.

Among these protein kinases, there may be mentioned more particularly protein kinase IKK-alpha (IKKα) and IKK-beta (IKKβ).

The compounds of the present invention are kinase inhibitors, in particular IKK-alpha and IKK-beta inhibitors, and consequently inhibit the NF-KB (nuclear factor kappa B) activity; they may thus be used in the treatment or the prophylaxis of inflammatory diseases, in cancer and diabetes.

NF-kB (Nuclear factor kappa B) belongs to a family of complexes of transcription factors consisting of various combinations of polypeptides Rel/NF-KB. Members of this family of polypeptides linked to NF-KB regulate the expression of genes involved in immune and inflammatory responses (Bames P J, Karin M (1997) N Engl J Med 336, 1066-1071) and (Baeuerle P A, Baichwal V R (1997) Adv Immunol 65, 111-137)). Under basal conditions, the NF-KB dimers are retained in the inactive form in the cytoplasm by inhibitory proteins which are members of the IKB family (Beg et al., genes Dev., 7:2064-2070, 1993; Gilmore and Morin, Trends Genet. 9:427-433), 1993); Haskil et al., Cell 65: 1281-1289, 1991). The proteins of the IKB family mask the signal for nuclear translocation of NF-KB. The stimulation of the cell by various types of ligands such as cytokines, the anti-CD40 ligand, lipopolysaccharides (LPS), oxidants, mitogens such as phorbol ester, viruses and many other stimulants, causes the activation of the IKB-kinase (IKK) complex which will in turn phosphorylate IKB at the level of the serine 32 and 34 residues. Once phosphorylated, IKB will be subject to ubiquitinations leading to its degradation by the proteasome (26S), thus allowing the release and translocation of NF-KB in the nucleus where it will bind to specific sequences at the level of the target gene promoters, thus inducing their transcription.

In the IKB-kinase (IKK) complex, the principal kinases are IKK1 (IKKα) and IKK2 (IKKβ) which are capable of directly phosphorylating the various classes of IKB. In this IKK complex, IKK2 is the dominant kinase (Mercurio et al., Mol. Cell. Biol., 19:1526, 1999, Zandi et al., Science; 28 1: 1 3) 60, 1998; Lee et al., Proc. Natl. Acad. Sci. USA 95:93) 19, 1998).

Among the genes regulated by NF-KB, many encode pro-inflammatory mediators, cytokines, cell adhesion molecules, acute phase proteins, which will in turn induce the activation of NF-KB by autocrine or paracrine mechanisms.

The inhibition of the activation of NF-KB appears to be very important in the treatment of inflammatory diseases.

In addition, NF-KB plays a role in the growth of normal cells but also of malignant cells.

The proteins produced by the expression of genes regulated by NF-KB comprise cytokines, chemokines, adhesion molecules, mediators of cell growth and mediators of angiogenesis. Moreover, various studies have shown that NF-KB plays an essential role in neoplastic transformations. For example, NF-KB may be associated with the transformation of cells in vitro and in vivo following overexpression, amplification, rearrangement or translocation events (Mercurio, R, and Manning, A. M. (1999) Oncogene, 18: 6163-6171). In some human lymphoid tumor cells, the genes encoding the various NF-KB members are rearranged or amplified. It has been shown that NF-KB may promote cell growth by inducing the transcription of cyclin D, which, when associated with the hyperphosphorylation of Rb, causes the g1 to S phase transition and the inhibition of apoptosis.

It has been shown that in a large number of tumor cell lines, a constitutive NF-KB activity is present following the activation of IKK2. NF-KB is constitutively activated in Hodgkin's diseases and the inhibition of NF-KB blocks the growth of these lymphomas. Moreover, the inhibition of NF-KB by the expression of the IKBa repressor induces the apoptosis of cells expressing the oncogenic allele H-Ras (Baldwin, J. Clin. Invest., 107:241 (2001), Bargou et al., J. Clin. Invest., 100:2961 (1997), Mayo et al., Science 178:1812 (1997).

The constitutive activity of NF-KB appears to contribute to oncogenesis through the activation of several anti-apoptotic genes such as Al/Bfi-1, IEX-1, MAP, which thus causes the suppression of the cell death pathway. Through the activation of cyclin D, NF-KB may promote the growth of tumor cells. The regulation of adhesion molecules and of surface proteases suggest a role for NF-KB signaling in metastases.

NF-KB is involved in the induction of chemoresistance. NF-KB is activated in response to a certain number of chemotherapy treatments. It has been shown that the inhibition of NF-KB by the use of the super-repressor form of IKBa in parallel with the chemotherapy treatment increases the efficacy of the chemotherapy in the xenograft models.

The subject of the present invention is products of formula (I):

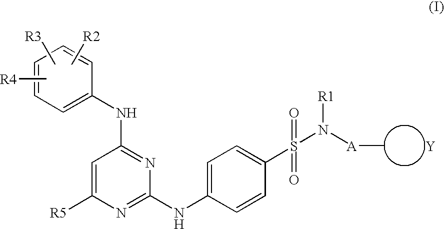

in which:
R2, R3 and R4, which are identical or different, are such that one represents a halogen atom and the other two, which are identical or different, represent a hydrogen atom or a halogen atom or an alkyl radical or an alkoxy radical;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9, the alkyl radicals which R1 represents being further optionally substituted with a 5-membered saturated or unsaturated heterocyclic radical attached by a carbon atom and optionally substituted with one or more radicals chosen from halogen atoms and alkyl or alkoxy radicals;
A represents a single bond or a radical —CH2-CO—NR6-, and R6, which is identical to or different than R1, is chosen from the values of R1;
the ring containing Y (or ring (Y)) consisting of 4 to 8 members and being saturated or partially saturated, with Y representing an oxygen atom O, a sulfur atom S optionally oxidized with one or two atoms of oxygen or a radical chosen from N—R7, C═O or its dioxolane as protecting group for the carbonyl functional group, CF2, CH—OR8 or CH—NR8R9;
it being understood that the ring containing Y (or ring (Y)), when Y represents NR7, may contain a carbon bridge consisting of 1 to 3 carbons,
R7 represents the hydrogen atom, a cycloalkyl radical or an alkyl, CH2-alkenyl or CH2-alkynyl radical, all optionally substituted with a naphthyl radical or with one or more radicals, which are identical or different, chosen from halogen atoms and hydroxyl, alkoxy, phenyl and heteroaryl radicals, the alkyl radicals which R7 represents being further optionally substituted with a phosphonate radical, with an alkylthio radical optionally oxidized to a sulfone or with an optionally substituted heterocycloalkyl radical,
R8 represents the hydrogen atom or the alkyl, cycloalkyl or heterocycloalkyl radicals which are themselves optionally substituted with one or more radicals chosen from halogen atoms and hydroxyl, alkoxy, NH2, NHalkyl or N(alkyl)2 radicals, the alkyl radicals which R8 represents being further optionally substituted with an alkylthio radical, with an optionally substituted phenyl radical or with an optionally substituted saturated or unsaturated heterocyclic radical,
NR8R9 is such that either R8 and R9, which are identical or different, are chosen from the values of R8, or R8 and R9 form with the nitrogen atom to which they are attached, a cyclic amine which may optionally contain one or two other heteroatoms chosen from O, S, N or NR10, the cyclic amine thus formed being itself optionally substituted with one or more alkyl radicals;
all the above heterocyclic, heterocycloalkyl and heteroaryl radicals consisting of 4 to 10 members (unless specified) and containing 1 to 3 heteroatoms chosen, where appropriate, from O, S, N and NR10;
all the above naphthyl, phenyl, heterocyclic, heterocycloalkyl and heteroaryl radicals being themselves optionally substituted with one or more radicals, which are identical or different, chosen from halogen atoms and hydroxyl, alkoxy, alkyl, hydroxyalkyl, alkoxyalkyl, CN, CF3, NH2, NHalk or N(alk)2 radicals;
R10 represents a hydrogen atom or an alkyl radical,
said products of formula (I) being in all the possible racemic, enantiomeric and diastereoisomeric isomer forms as well as the addition salts with inorganic and organic acids of said products of formula (I).

Among the products of formula (I) as defined above, in which R1, R2, R3, R4, R5 and A have the meanings indicated above, there may be mentioned particularly those in which the ring (Y) is chosen from the following definitions:
when ring (Y) is such that Y represents C—OH, CF2, CH—OR8 or CH—NR8R9, the ring formed may in particular be a cyclobutyl, cyclopentyl, cyclohexyl or cycloheptyl and particularly a cyclohexyl, these radicals being therefore substituted in particular at the para-position respectively with OH, 2 F, the radial OR8 or the radical NR8R9 in which R8 and R9 are chosen from the meanings defined above;
when ring (Y) is such that Y represents NR7, the ring formed may in particular be an azetidine, pyrrolidine or piperidine radical with the nitrogen atom N at the para or meta position, which therefore carries the substituent R7 as defined above. When ring (Y), such that Y represents NR7, contains a carbon-based bridge consisting of 1 to 3 carbons, the ring formed may in particular be the ring 8-(azabicyclo(3.2.1)oct-3-yl) or alternatively a ring chosen from the following: N,9-dimethyl-9-azabicyclo[3.3.1]nona-3-yl, N,6-dimethyl-6-azabicyclo[3.2.1]octan-3-yl, N,3-dimethyl-3-azabicyclo[3.2.1]octan-8-yl or alternatively N,3-dimethyl-3-azabicyclo[3.3.1]nonan-9-yl;
when ring (Y) is such that Y represents S, the ring formed may in particular be a tetrahydro-2H-thiopyran or a tetrahydro-2H-thiofuran; when ring (Y) is such that Y represents SO2, the ring formed may in particular be a dioxidotetrahydro-3-thienyl;
when ring (Y) is such that Y represents O, the ring formed may in particular be a tetrahydrofuran or tetrahydropyran. When ring (Y) is such that Y represents the dioxolane of C═O, the ring formed may in particular be dioxaspiro(4,5)dec-8-yl.

The present invention relates particularly to the products of formula (I) as defined above in which R2, R3, R4, R5, A and ring (Y) have the meanings indicated above and R1 represents a hydrogen atom or a linear or branched alkyl radical containing from 1 to 5 carbon atoms or alternatively R1 represents this alkyl radical substituted with a saturated or unsaturated, preferably monocyclic 5-membered heterocycle which is itself optionally substituted as indicated above.

The present invention relates particularly to the products of formula (I) as defined above in which R2, R3, R4, R5 and A have the meanings indicated above, R1 represents a hydrogen atom or a linear or branched optionally substituted alkyl radical containing from 1 to 4 carbon atoms and in particular CH3 and ring (Y) is such that Y represents NR7 with R7 represents an alkyl radical containing from 1 to 6 linear or branched carbon atoms substituted with a radical chosen from hydroxyl, CF3, phosphonate, sulfone, phenyl and saturated or unsaturated monocyclic or bicyclic heterocyclic radicals, these phenyl and heterocyclic radicals being themselves optionally substituted as indicated above.

The present invention relates most particularly to the products of formula (I) as defined above in which R2, R3, R4, R5 and A have the meanings indicated above, R1 represents a linear or branched alkyl radical containing from 1 to 4 carbon atoms and in particular CH3, and ring (Y) is such that Y represents NR8R9 in which R8 represents a hydrogen atom or CH3 and R9 represents a linear or branched alkyl radical containing from 1 to 6 carbon atoms substituted with a radical chosen from the hydroxyl, CF3, phosphonate, sulfone, phenyl and saturated or unsaturated, monocyclic or bicyclic heterocyclic radicals, these phenyl and heterocyclic radicals being themselves optionally substituted as indicated above: in particular R9 represents a linear or branched alkyl radical containing from 1 to 4 carbon atoms in particular CH3 or C2H5 substituted with a saturated or unsaturated, preferably monocyclic 5-membered heterocycle which is itself optionally substituted as indicated above.

The present invention thus relates in particular to the products of formula (I) as defined above in which R2, R3, R4, R5 and A are chosen from the meanings indicated above and the other substituents are chosen from the preferred values defined as follows:
R1 represents a hydrogen atom, a CH3 radical or a linear or branched alkyl radical containing from 1 to 4 carbon atoms optionally substituted with a radical NH2, NHalk, N(alk)2 or with a saturated or unsaturated heterocycle, preferably a 5-membered monocycle such as pyrrolidine and ring (Y) represents a piperidine substituted on its nitrogen atom with R7 which represents an alkyl radical carrying a phosphonate;
R1 is chosen from the values defined above and ring (Y) represents a cyclohexyl radical substituted with a radical NR8R9 as defined above;
R1 represents a CH3 radical optionally substituted with a saturated or unsaturated heterocycle as defined above and R7 represents a CH3 radical;
R1 represents a hydrogen atom or a CH3 radical and ring (Y) represents a piperidine or a ring 8-azabicyclo(3.2.1)oct-3-yl substituted on their nitrogen atom with R7, with R7 as defined above.

Among the products of formula (I) as defined above, in which R1, R2, R3, R4, R5 and A have the meanings indicated above, there may be mentioned for example those in which ring (Y) is chosen from the following definitions:
ring (Y) such that Y represents —N—R7, with R7 representing H;
ring (Y) such that Y represents —N—R7, with R7 representing CH3;
ring (Y) such that Y represents —N—R7, with R7 representing cycloalkyl such as in particular cyclopropyl;
ring (Y) such that Y represents —N—R7, with R7 representing an alkyl radical, in particular CH3, C2H5 or C3H7 substituted with a phosphonate;
ring (Y) such that Y represents —N—R7, with R7 representing an alkyl radical, in particular CH3, C2H5 or C3H7, substituted with an alkylthio such as S—CH3 or S—C2H5 with S optionally oxidized to a sulfone in order to form for example SO2-CH3 or SO2-C2H5;
ring (Y) such that Y represents —N—R7, with R7 representing alkyl such as in particular CH3 or C2H5 substituted with one or more radicals chosen from halogen atoms such as in particular F, and the radicals phenyl and mono- or bicyclic heterocycle, phenyl and heterocycles which are themselves optionally substituted with one or more radicals chosen from halogen atoms and the radicals alkyl, alkoxy, OH, CN, CF3, NH2, NHalk and N(alk)2: among these heterocycles which R7 carries, there may be mentioned in particular the unsaturated 5-membered heterocycles containing one to three heteroatoms chosen from N, O and S: thus R7 may represent in particular the radicals —CH2-thienyl, —CH2-thiazole (N,S), —CH2-thiadiazole (N,N,S), CH2-furan (O), —CH2-pyrazole (N,N), —CH2-isoxazole (N,O), —CH2-pyrrole (NH, NCH3), these radicals, in particular pyrazole, isoxazole or pyrrole, being themselves optionally substituted in particular with alkyl containing from 1 to 3 carbon atoms such as in particular CH3 or C2H5.

R7 may also carry heterocycles as defined above such as the radicals pyridin-(with N of the pyridine at 3 different positions); 2,3-dihydro-1H-indole; quinoline; isoquinoline; pyrimidine; 2,3-dihydrobenzofuran; ([1,8]naphthyridin-; pyridine N-oxide; 4-[(benzo-[1.2.5]oxadiazole; (2,3-dihydrobenzofuran.
ring (Y) such that Y represents CH—NR8R9 with NR8R9 such that R8 represents a hydrogen atom or an alkyl radical such as in particular CH3, and R9 represents a linear or branched alkyl radical such as in particular CH3, C2H5 or —CH2- or —CH(CH3)- or —CH(CH3)-CH2-substituted either with a saturated or unsaturated mono- or bicyclic heterocycle optionally substituted either with an optionally substituted phenyl radical. Among the heterocycles which R9 carries, there may be mentioned in particular the following radicals: pyridine (with N of the pyridine at 3 different positions); 2,3-dihydro-1H-indole; quinoline; isoquinoline; pyrimidine; 2,3-dihydrobenzofuran; ([1,8]naphthyridine; 4-[(benzo[1.2.5]-oxadiazole; (2,3-dihydrobenzofuran.

Such heterocycles are optionally substituted with one or more radicals as defined above and chosen in particular from the radicals CH3, CN, NH2, NHCH3. The phenyl radical is optionally substituted with one or more radicals chosen in particular from OH and CF3.

Among the products of formula (I) as defined above, in which R2, R3, R4, R5, A and ring (Y) have the meanings indicated above, there may be mentioned for example those in which R1 is chosen from the following definitions:
R1 represents H
R1 represents CH3
R1 represents alkenyl (3C) radicals such as allyl or alkynyl (3C) radicals such as propargyl
—R1 represents alkyl and in particular CH3, C2H5, C3H7 substituted with a radical chosen from NH2, NH(alk), N(alk)2, NH—CH2-CH2OH, NH—CH2-C3H7-OH, NH(CH2-CF3), alkoxy, OH, or a saturated heterocycle such as for example pyrrolidine, tetrahydrofuran or an unsaturated heterocycle such as in particular those defined above for R7: thus R1 may in particular represent the radicals —CH2-thienyl, —CH2-thiazole (N,S), —CH2-thiadiazole (N,N,S), CH2-furan (O), —CH2-pyrazole (N,N), —CH2-isoxazole (N,O), —CH2-pyrrole (NH, NCH3), these radicals, in particular pyrazole, isoxazole or pyrrole, being themselves optionally substituted in particular with alkyl containing from 1 to 3 carbon atoms such as in particular CH3 or C2H5 to give for example —CH2-pyrazole-CH3, —CH2-isoxazole-CH3, —CH2-pyrrole-CH3.

The subject of the present invention is thus the products of formula (I):

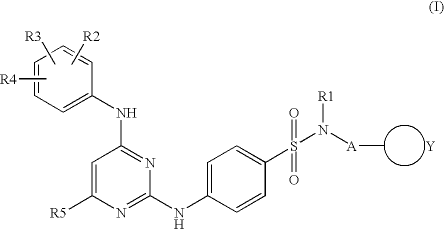

in which:
R2, R3 and R4, which are identical or different, are such that one represents a halogen atom and the other two, which are identical or different, represent a hydrogen atom or halogen atom or an alkyl radical;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9;
A represents a single bond or a radical —CH2-CO—NR6-, and R6, which is identical to or different than R1, is chosen from the values of R1;
the ring containing Y consisting of 4 to 8 members and being saturated or partially saturated, with Y representing an oxygen atom O, a sulfur atom S optionally oxidized with one or two oxygen atoms or a radical chosen from N—R7, C═O, CF2, CH—OR8 or CH—NR8R9;
R7 represents a hydrogen atom or an alkyl, CH2-alkenyl or CH2-alkynyl radical, all optionally substituted with a naphthyl radical or with one or more radicals which are identical or different, chosen from halogen atoms and hydroxyl, phenyl and heteroaryl radicals, all these naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals;
the heteroaryl radicals consisting of 5 to 10 members and containing 1 to 3 heteroatoms chosen from O, S, N and NR10;
R8 represents the hydrogen atom or alkyl, cycloalkyl or heterocycloalkyl radicals which are themselves optionally substituted with one or more radicals chosen from hydroxyl, alkoxy, NH2, Nalkyl or N(alkyl)2 radicals;
NR8R9 is such that either R8 and R9, which are identical or different, are chosen from the values of R8 or R8 and R9 form with the nitrogen atom to which they are attached a cyclic amine which may optionally contain one or two other heteroatoms chosen from O, S, N or NR10;
R10 represents a hydrogen atom or an alkyl radical; said products of formula (I) being in all the possible racemic, enantiomeric and diastereoisomeric isomer forms, as well as the addition salts with inorganic and organic acids of said products of formula (I).

The subject of the present invention is thus the products of formula (I):

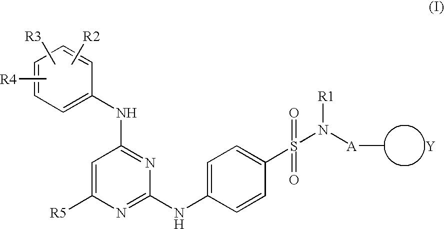

in which:
R2, R3 and R4, which are identical or different, are such that one represents a halogen atom and the other two, which are identical or different, represent a hydrogen atom or a halogen atom;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9;
A represents a single bond or a radical —CH2-CO—NR6-, and R6, which is identical to or different than R1, is chosen from the values of R1;
the ring containing Y consisting of 4 to 8 members and being saturated or partially saturated, with Y representing an oxygen atom O, a sulfur atom S optionally oxidized with one or two oxygen atoms or a radical chosen from N—R7, C═O, CF2, CH—OR8 or CH—NR8R9;
R7 represents a hydrogen atom or an alkyl, CH2-alkenyl or CH2-alkynyl radical, all optionally substituted with a naphthyl radical or with one or more radicals, which are identical or different, chosen from halogen atoms and phenyl and heteroaryl radicals, all these naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals;
the heteroaryl radicals consisting of 5 to 10 members and containing 1 to 3 heteroatoms chosen from O, S, N and NR10;
R8 represent the hydrogen atom or alkyl, cycloalkyl or heterocycloalkyl radicals which are themselves optionally substituted with one or more radicals chosen from hydroxyl, alkoxy, NH2, Nalkyl or N(alkyl)2 radicals;
NR8R9 is such that either R8 and R9, which are identical or different, are chosen from the values of R8, or R8 and R9 form with the nitrogen atom to which they are attached a cyclic amine which may optionally contain one or two other heteroatoms chosen from O, S, N or NR10;
R10 represents a hydrogen atom or an alkyl radical;
said products of formula (I) being in all the possible racemic, enantiomeric and diastereoisomeric isomer forms, as well as the addition salts with inorganic and organic acids of said products of formula (I).

In the products of formula (I) and in the text that follows, the terms indicated have the following meanings:
the term halogen denotes fluorine, chlorine, bromine or iodine atoms, and preferably fluorine, chlorine or bromine atoms;
the term alkyl radical denotes a linear or branched radical containing at most 6 carbon atoms and in particular methyl, ethyl, propyl, isopropyl, n-butyl, isobutyl, sec-butyl, tert-butyl, pentyl, isopentyl, sec-pentyl, tert-pentyl, neopentyl, hexyl, isohexyl, sec-hexyl, tert-hexyl radicals and their linear or branched position isomers;
the term hydroxyalkyl radical denotes the alkyl radicals indicated above substituted with at least one hydroxyl radical;
the term alkenyl radical denotes a linear or branched radical containing at most 6 carbon atoms and preferably 4 carbon atoms, chosen for example from the following values: ethenyl or vinyl, propenyl or allyl, 1-propenyl, n-butenyl, i-butenyl, 3-methylbut-2-enyl, n-pentenyl, hexenyl and their linear or branched position isomers: among the alkenyl values, the allyl or butenyl values may be mentioned in particular;
the term alkynyl radical denotes a linear or branched radical containing at most 6 carbon atoms and preferably 4 carbon atoms, chosen for example from the following values: ethynyl, propynyl or propargyl, butynyl, n-butynyl, i-butynyl, 3-methylbut-2-ynyl, pentynyl or hexynyl and their linear or branched position isomers: among the alkynyl values, the propargyl value may be mentioned more particularly;
the term alkylene radical denotes a linear or branched bivalent radical containing at most 12 carbon atoms, derived from the alkyl radical above and thus chosen for example from methylene, ethylene, propylene, isopropylene, butylene, isobutylene, sec-butylene and pentylene radicals;
the term alkoxy radical denotes a linear or branched radical containing at most 12 carbon atoms and preferably 6 carbon atoms, chosen for example from methoxy, ethoxy, propoxy, isopropoxy, linear, secondary or tertiary butoxy, pentoxy, hexoxy and heptoxy radicals and their linear or branched position isomers;
the term cycloalkyl radical denotes a monocyclic or bicyclic carbocyclic radical containing from 3 to 7 rings and denotes in particular cyclopropyl, cyclobutyl, cyclopentyl, cyclohexyl and cycloheptyl radicals;
the term —O-cycloalkyl radical denotes a radical in which the cycloalkyl radical has the meaning indicated above;
the term aryl radical denotes unsaturated radicals which are monocyclic or consist of fused, carbocyclic rings. As examples of such an aryl radical, mention may be made of phenyl or naphthyl radicals;
the term heterocyclic radical denotes a saturated carbocyclic (heterocycloalkyl) radical or a partially or totally unsaturated carbocyclic (heteroaryl) radical consisting of 4 to 10 members interrupted by one or 3 heteroatoms, which are identical or different, chosen from oxygen, nitrogen or sulfur atoms.

Among the 5-membered heteroaryl radicals, there may be mentioned furyl, 2-furyl, pyrrolyl, thiazolyl, isothiazolyl, diazolyl, thiadiazolyl, 1,3,4-thiadiazolyl, oxazolyl, oxadiazolyl, isoxazolyl, 3-isoxazolyl, 4-isoxazolyl, imidazolyl, pyrazolyl, thienyl, 2-thienyl or 3-thienyl radicals, or triazolyl groups.

Among the 6-membered heteroaryl radicals, there may be mentioned in particular pyridyl radicals such as 2-pyridyl, 3-pyridyl and 4-pyridyl, and pyrimidyl, pyrimidinyl, pyridazinyl, pyrazinyl, pyrazinyl, pyridazinyl, pyridyl and pyrimidinyl radicals.

As fused heteroaryl radicals containing at least one heteroatom chosen from sulfur, nitrogen and oxygen, there may be mentioned for example benzothienyl such as 3-benzothienyl, benzofuryl, benzofuranyl, benzimidazolyl, benzoxazolyl, indolyl, quinolyl, isoquinolyl, azaindolyl and naphthyridinyl.

Among the fused heteroaryl radicals, there may be mentioned more particularly the benzothienyl, benzofuranyl, indolyl, benzimidazolyl, benzothiazolyl, naphthyridinyl, indazolyl, quinolyl such as 4-quinolyl, 5-quinolyl, isoquinolyl, azaindolyl such as 4-azaindolyl, 3-azaindolyl, imidazo(4,5)pyridine, indolizinyl and quinazolinyl radicals.
The amino NH2 radical may be substituted with one or two radicals, which are identical or different, in particular chosen from alkyl, cycloalkyl and heterocycloalkyl radicals as defined above, to give in particular alkylamino, NHalk, dialkylamino N(alk)2, cycloalkylamino, alkylcycloalkylamino, heterocyclo-alkylamino or alternatively alkylheterocycloalkylamino radicals in which the alkyl, cycloalkyl or heterocycloalkyl radicals are optionally substituted in particular with one or more radicals, which are identical or different, chosen from hydroxyl, alkoxy, NH2, Nalkyl, N(alkyl)2;
the terms alkylamino or NH(alk) radical and dialkylamino or N(alk)2 radical thus denotes amino radicals substituted respectively with one or two linear or branched alkyl radicals, which are identical or different in the case of dialkylamino, chosen from alkyl radicals as defined above: there may be mentioned for example methylamino, ethylamino, propylamino or butylamino radicals, dimethylamino, diethylamino, methylethylamino radicals;
the term cycloalkylamino radical thus denotes an amino radical substituted in particular with a cycloalkyl radical chosen from the radicals defined above: there may thus be mentioned for example cyclopropylamino, cyclobutylamino, cyclopentylamino or alternatively cyclohexylamino radicals;
the term cyclic amine denotes a monocyclic or bicyclic radical containing from 3 to 10 members in which at least one carbon atom is replaced with a nitrogen atom, it being possible for this cyclic radical to also contain one or more other heteroatoms chosen from O, S, SO2, N or NR10 with R10 as defined above: as examples of such cyclic amines, there may be mentioned for example pyrrolyl, piperidyl, morpholinyl, piperazinyl, pyrrolidinyl, azetidinyl radicals. There may be mentioned more particularly piperidinyl, morpholinyl, piperazinyl or azetidinyl radicals.

The term patient denotes human beings but also other mammals.

The term “Prodrug” denotes a product which may be converted in vivo by metabolic mechanisms (such as hydrolysis) to a product of formula (I). For example, an ester of a product of formula (I) containing a hydroxyl group may be converted by hydrolysis in vivo to its parent molecule.

There may be mentioned, by way of examples, esters of products of formula (I) containing a hydroxyl group such as acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexyl-sulfamates and quinates.

Particularly useful esters of products of formula (I) containing a hydroxyl group may be prepared from acid residues such as those described by Bundgaard et al., J. Med. Chem., 1989, 32, page 2503-2507: these esters include in particular substituted (aminomethyl)benzoates, dialkylaminomethylbenzoates in which the two alkyl groups may be linked together or may be interrupted by an oxygen atom or by an optionally substituted nitrogen atom, that is an alkylated nitrogen atom or alternatively (morpholino-methyl)benzoates, e.g. 3- or 4-(morpholinomethyl)benzoates, and (4-alkylpiperazin-1-yl)benzoates, e.g. 3- or 4-(4-alkylpiperazin-1-yl)benzoates.

When the products of formula (I) contain an amino radical salifiable with an acid, it is clearly understood that these acid salts also form part of the invention. There may be mentioned for example the salts obtained with hydrochloric or methanesulfonic acids.

The addition salts with inorganic or organic acids of the products of formula (I) may be, for example, the salts formed with hydrochloric, hydrobromic, hydroiodic, nitric, sulfuric, phosphoric, propionic, acetic, trifluoroacetic, formic, benzoic, maleic, fumaric, succinic, tartaric, citric, oxalic, glyoxylic, aspartic and ascorbic acids, alkylmonosulfonic acids such as for example methanesulfonic acid, ethanesulfonic acid, propanesulfonic acid, alkyldisulfonic acids such as for example methanedisulfonic acid, alpha-, beta-ethanedisulfonic acid, arylmonosulfonic acids such as benzenesulfonic acid and aryldisulfonic acids.

It may be recalled that the stereoisomerism may be defined in its broad sense as the isomerism of compounds having the same structural formulae but whose different groups are arranged differently in space, such as in particular in monosubstituted cyclohexanes whose substituent may be at the axial or equatorial position. However, another type of stereoisomerism exists, due to the different spatial arrangements of substituents attached either to double bonds, or to rings, which is often called E/Z geometric isomerism or cis-trans isomerism or diastereoisomer. The term stereoisomer is used in the present application in its broadest sense and therefore relates to all the compounds indicated above.

The subject of the present invention is in particular the products of formula (I) above in which:

R2, R3 and R4, which are identical or different, are such that one represents a fluorine or chlorine atom and the other two, which are identical or different, represent a hydrogen atom or a fluorine or chlorine atom;
R5 represents a hydrogen atom or a fluorine or chlorine atom;
R1 represents a hydrogen atom, a cycloalkyl radical or an alkyl radical optionally substituted with one or more radicals, which are identical or different, chosen from the fluorine atom, OR8 and NR8R9;
A represents a single bond or a radical —CH2-CO—NR6-, and R6 representing a hydrogen atom or a linear or branched alkyl radical containing at most 4 carbon atoms;
the ring containing Y consisting of 4 to 7 members and being saturated or partially saturated, with Y representing an oxygen atom O, a sulfur atom S optionally oxidized with one or two oxygen atoms or a radical chosen from N—R7, C═O, CF2, CH—OR8 or CH—NR8R9;
R7 represents a hydrogen atom or an alkyl radical optionally substituted with one or more radicals, which are identical or different, chosen from halogen atoms and phenyl and heteroaryl radicals, the phenyl and heteroaryl radicals themselves being optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals;
the heteroaryl radicals consisting of 5 to 7 members and containing 1 to 3 heteroatoms chosen from O, S, N and NR10;
R8 represents the hydrogen atom, linear or branched alkyl radicals containing at most 4 carbon atoms or cycloalkyl radicals containing 3 to 6 members, alkyl and cycloalkyl radicals which are themselves optionally substituted with a hydroxyl radical;
NR8R9 is such that either R8 and R9, which are identical or different, are chosen from the values of R8, or R8 and R9 form with the nitrogen atom to which they are attached a cyclic amine chosen from pyrrolyl, piperidyl, morpholinyl, pyrrolidinyl, azetidinyl and piperazinyl radicals, optionally substituted on its second atom with an alkyl radical;
said products of formula (I) being in all the possible racemic, enantiomeric and diastereoisomeric isomer forms, and the addition salts with inorganic or organic acids of said products of formula (I).

The subject of the present invention is in particular the products of formula (I) as defined above in which

R2, R3 and R4, which are identical or different, are such that one represents a fluorine atom and the other two, which are identical or different, represent a hydrogen atom or a fluorine or chlorine atom;
R5 represents a hydrogen atom or a chlorine atom;
R1 represents a hydrogen atom, a cycloalkyl radical or an alkyl radical optionally substituted with one or more radicals chosen from the fluorine atom and hydroxyl, amino, methylamino, dimethylamino, piperidinyl, morpholinyl, azetidinyl or piperazinyl radicals;
A represents a single bond or a radical —CH2-CO—NR6-, and R6 representing a hydrogen atom or an alkyl radical containing at most 1 or 2 carbon atoms;
the ring containing Y consisting of 4 to 7 members, being saturated, with Y representing an oxygen atom O, a sulfur atom S optionally oxidized with one or two oxygen atoms or a radical chosen from N—R7, CH—NH2, CH—NHalk or CH—N(alk)2;
R7 represents a hydrogen atom or an alkyl radical optionally substituted with a phenyl, pyridyl, thienyl, thiazolyl, pyrazinyl, furyl or imidazolyl radical which are themselves optionally substituted with one or more radicals chosen from halogen atoms and hydroxyl, methoxy, methyl, hydroxymethyl, methoxymethyl, trifluoromethyl, amino, methylamino and dimethylamino radicals;
said products of formula (I) being in all the possible racemic, enantiomeric and diastereoisomeric isomer forms, and the addition salts with inorganic and organic acids of said products of formula (I).

The subject of the present invention is in particular the products of formula (I) as defined above in which R2, R3 and R4, which are identical or different, are such that one represents a fluorine atom and the other two represent, one a hydrogen atom and the other a fluorine or chlorine atom or a methyl radical;

R5 represents a hydrogen atom or a chlorine atom;
R1 represents a hydrogen atom; a cyclopropyl radical; a methyl radical; or an ethyl, propyl or butyl radicals optionally substituted with the fluorine atom or a hydroxyl radical or an amino, alkylamino, dialkylamino or pyrrolidinyl radical;
A represents a single bond, —CH2-CO—NH— or —CH2-CO—NCH3- and the ring containing Y is chosen from cyclohexyl radicals, itself optionally substituted with amino; tetrahydropyran; dioxydothienyl; and the pyrrolidinyl, piperidinyl and azepinyl radicals optionally substituted on their nitrogen atom with a methyl, propyl, isopropyl, isobutyl, isopentyl or ethyl radical, themselves optionally substituted with one or more radicals chosen from halogen atoms and a hydroxyl radical, a phenyl radical which is itself optionally substituted with one or more halogen atoms, a quinolyl radical, a pyridyl radical which is optionally oxidized on its nitrogen atom, a thienyl radical, a thiazolyl radical, a pyrazinyl radical, a furyl radical and an imidazolyl radical which is itself optionally substituted with alkyl;
said products of formula (I) being in all the possible racemic, enantiomeric and diastereoisomeric isomer forms, and the addition salts with inorganic and organic acids of said products of formula (I).

The subject of the present invention is in particular the products of formula (I) as defined above in which R2, R3 and R4, which are identical or different, are such that one represents a fluorine atom and the other two represent, one a hydrogen atom and the other a fluorine or chlorine atom or a methyl radical;

R5 represents a hydrogen atom;
R1 represents a methyl radical; or an ethyl radical, optionally substituted with an amino, alkylamino, dialkylamino or pyrrolidinyl radical;
A represents a single bond and the ring containing Y represents a cyclohexyl radical which is itself optionally substituted with amino or a piperidinyl radical optionally substituted on its nitrogen atom with a methyl, propyl, isopropyl, isobutyl, isopentyl or ethyl radical, which are themselves optionally substituted with one or more halogen atoms or a radical chosen from hydroxyl; phenyl which is itself optionally substituted with halogen; quinolyl; pyridyl optionally oxidized on its nitrogen atom; furyl; and imidazolyl which is itself optionally substituted with alkyl;
said products of formula (I) being in all the possible racemic, enantiomeric and diastereoisomeric isomer forms, and their addition salts with inorganic and organic acids of said products of formula (I).

The subject of the present invention is in particular the products of formula (I) above in which

R2, R3 and R4, which are identical or different, are such that one represents a fluorine atom and the other two represent, one a hydrogen atom and the other a fluorine or chlorine atom;
R5 represents a hydrogen atom or a chlorine atom;
R1 represents a hydrogen atom; a cyclopropyl radical; a methyl radical; or an ethyl, propyl or butyl radical optionally substituted with the fluorine atom or a hydroxyl radical or a dialkylamino radical;
A represents a single bond, —CH2-CO—NH— or —CH2-CO—NCH3- and the ring containing Y is chosen from tetrahydropyran and dioxidothienyl radicals and pyrrolidinyl, piperidyl and azepinyl radicals optionally substituted on their nitrogen atom with a methyl or ethyl radical, which are themselves optionally substituted with a phenyl, pyridyl, thienyl, thiazolyl, pyrazinyl, furyl or imidazolyl radical;
said products of formula (I) being in all the possible racemic, enantiomeric and diastereoisomeric isomer forms, and the addition salts with inorganic and organic acids of said products of formula (I).

In the products of formula (I) according to the present invention, when R1 represents hydrogen, then A preferably represents the radical —CH2-CO—NR6 as defined above.

There may be mentioned particularly the products of formula (I) in which A represents a single bond, the other substituents R1, R2, R3, R4, R5 and ring (Y) of said products of formula (I) being chosen from the values indicated above.

There may thus be particularly mentioned the products of formula (I) in which R5 represents a hydrogen atom, the other substituents R1, R2, R3, R4, A and ring (Y) of said products of formula (I) being chosen from the values indicated above.

The products of formula (I) as defined above are preferred, in which when NR8R9 does not form a cyclic amine, then NR8R9 is such that R8 represents a hydrogen atom and R9 is chosen from all the values defined for R8.

When one of R2, R3, R4 represents alkoxy, methoxy is preferred.

The subject of the present invention is particularly the products of formula (I) corresponding to the following names:
2-{4-[4-(4-fluorophenylamino)pyrimidin-2-ylamino]-benzenesulfonylamino}-N-(tetrahydropyran-4-yl)-acetamide
4-[4-(4-fluorophenylamino)pyrimidin-2-ylamino]-N-methyl-N-(1-pyridin-2-ylmethylpiperidin-4-yl)benzene-sulfonamide
N-(2-dimethylaminoethyl)-4-[4-(4-fluorophenylamino)-pyrimidin-2-ylamino]-N-(1-methylpiperidin-4-yl)benzene-sulfonamide
4-[4-(4-fluorophenylamino)pyrimidin-2-ylamino]-N-(2-hydroxyethyl)-N-(1-methylpiperidin-4-yl)benzene-sulfonamide
4-[4-(4-fluorophenylamino)pyrimidin-2-ylamino]-N-methyl-N-(1-pyridyl-3-ylmethylpiperidin-4-yl)benzene-sulfonamide
4-[4-(4-fluorophenylamino)pyrimidin-2-ylamino]-N-piperidin-4-yl-N-(2-pyrrolidin-1-ylethyl)benzenesulfon-amide hydrochloride
N-(2-aminoethyl)-4-[4-(3-chloro-4-fluorophenylamino)-pyrimidin-2-ylamino]-N-piperidin-4-ylbenzenesulfonamide hydrochloride
   said products of formula (I) being in all the possible racemic, enantiomeric and diastereoisomeric isomer forms, and the addition salts with inorganic and organic acids of said products of formula (I).

The subject of the present invention is more particularly the products of formula (I) above corresponding to the following names:
2-{4-[4-(4-fluorophenylamino)pyrimidin-2-ylamino]-benzenesulfonylamino}-N-(tetrahydropyran-4-yl)acetamide
4-[4-(4-fluorophenylamino)pyrimidin-2-ylamino]-N-methyl-N-(1-pyridin-2-ylmethylpiperidin-4-yl)benzene-sulfonamide
N-(2-dimethylaminoethyl)-4-[4-(4-fluorophenylamino)-pyrimidin-2-ylamino]-N-(1-methylpiperidin-4-yl)benzene-sulfonamide
4-[4-(4-fluorophenylamino)pyrimidin-2-ylamino]-N-(2-hydroxyethyl)-N-(1-methylpiperidin-4-yl)benzenesulfon-amide
4-[4-(4-fluorophenylamino)pyrimidin-2-ylamino]-N-methyl-N-(1-pyridyl-3-ylmethylpiperidin-4-yl)benzene-sulfonamide
   said products of formula (I) being in all the possible racemic, enantiomeric and diastereoisomeric isomer forms, and the addition salts with inorganic and organic acids of said products of formula (I).

The subject of the present invention is also the processes for preparing the products of formula (I) as defined above.

The subject of the present invention is in particular the process for preparing the products of formula (I) as defined above, characterized in that a product of formula (II):

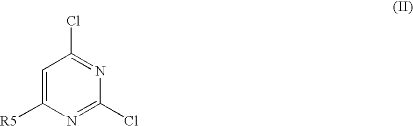

in which R5 has the meaning indicated above,
is reacted with a product of formula (III):

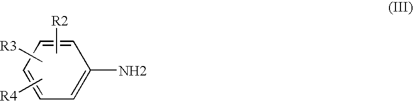

in which R2, R3 and R4 have the meanings indicated above, in order to obtain a product of formula (IV),

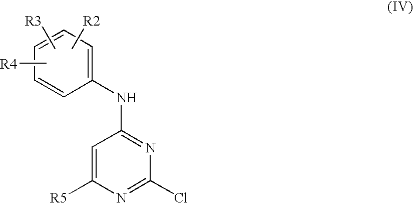

in which R2, R3, R4 and R5 have the meanings indicated above,
which product of formula (IV) is reacted with the aniline of formula (V):

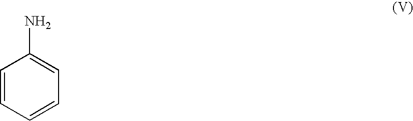

in order to obtain a product of formula (VI):

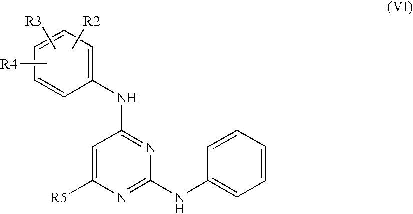

in which R2, R3, R4 and R5 have the meanings indicated above,
which product of formula (VI) is reacted with chlorosulfonic acid SO2(OH)Cl in order to obtain the corresponding product of formula (VII):

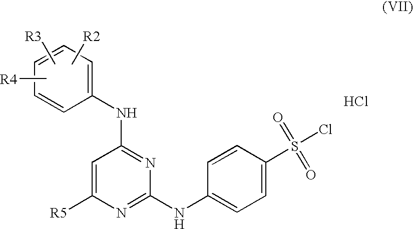

in which R2, R3, R4 and R5 have the meanings indicated above, which product of formula (VII) is reacted with an amine of formula (VIII):

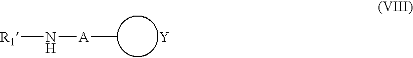

in which R₁′ has the meaning indicated above for R1, in which the reactive functional groups which may be present are optionally protected with protecting groups, in order to obtain a product of formula (I₁):

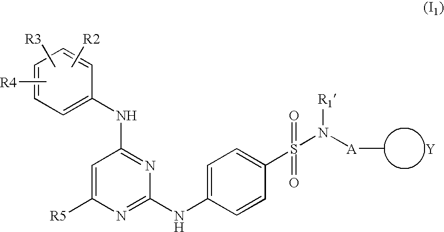

in which R₁′, R2, R3, R4 and R5 have the meanings indicated above,
which products of formula (I₁) may be products of formula (I) and which, in order to obtain products or other products of formula (I), may be subjected, if desired and if necessary, to one or more of the following conversion reactions in any order:
a) a reaction for oxidation of an alkylthio group to the corresponding sulfoxide or sulfone,
b) a reaction for conversion of an alkoxy functional group to a hydroxyl functional group, or alternatively of a hydroxyl functional group to an alkoxy functional group,
c) a reaction for oxidation of an alcohol functional group to an aldehyde or ketone functional group,
d) a reaction for removal of the protecting groups which the protected reactive functional groups may carry,
e) a reaction for salification with an inorganic or organic acid in order to obtain the corresponding salt,
f) a reaction for resolution of the racemic forms to resolved products,
said products of formula (I) thus obtained being in all the possible racemic, enantiomeric and diastereoisomeric isomer forms.

The subject of the present invention is also a process for preparing the products of formula (I) as defined above, in which Y represents the radical NR7 as defined above, with R7 representing CH2-RZ and RZ represents an alkyl, alkenyl or alkynyl radical, all optionally substituted with a naphthyl radical or with one or more radicals, which are identical or different, chosen from halogen atoms and phenyl and heteroaryl radicals, all these naphthyl, phenyl and heteroaryl radicals being themselves optionally substituted with one or more radicals, which are identical or different, chosen from halogen atoms and phenyl and heteroaryl radicals, all these naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals.

Such a process is characterized in that the compound of formula (A):

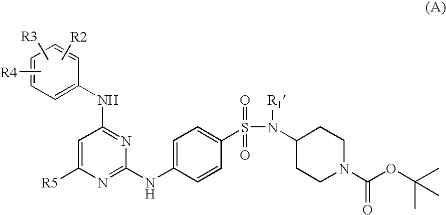

in which R₁′, R2, R3, R4 and R5 are the meanings indicated above,
is subjected to a reaction for deprotection of the carbamate functional group in order to obtain a product of formula (IX):

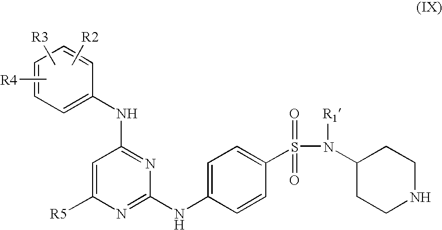

in which R₁′, R2, R3, R4 and R5 have the meanings indicated above, which product of formula (IX) is subjected to reductive amination conditions in the presence of the aldehyde of formula (X):

RZ′-CHO (X)

in which RZ′ has the meaning indicated above for RZ, in which the reactive functional groups which may be present are optionally protected with protecting groups, in order to obtain a product of formula (I₂):

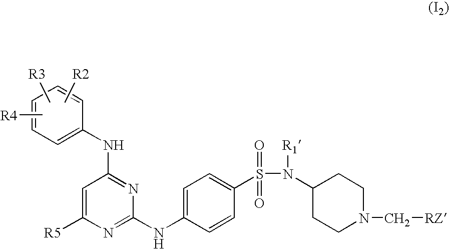

in which R₁′, R2, R3, R4, R5 and RZ′ have the meanings indicated above,
which products of formula (I₂) may be products of formula (I) and which, in order to obtain products or other products of formula (I), may be subjected, if desired and if necessary, in any order, to one or more conversion reactions a) to f) as defined above,
said products of formula (I₂) thus obtained being in all the possible racemic, enantiomeric and diastereoisomeric isomer forms.

Under preferred conditions for carrying out the invention, the processes described above may be carried out in the following manner:

The product of formula (II) is subjected to the action of the product of formula (III) as defined above, in particular in an alcohol such as for example butanol, propanol, ethanol or dimethylformamide between 80 and 140° C., in order to give a product of formula (IV) as defined above.

The product of formula (IV) thus obtained is subjected to the action of the aniline of formula (V) as defined above, in particular in an alcohol such as for example butanol or dimethylformamide, in the presence or absence of a strong acid (HCl) in a catalytic quantity under reflux conditions in order to give a product of formula (VI) as defined above.

The product of formula (VI) thus obtained is subjected to the action of chlorosulfonic acid in particular first at 0° C. and then at room temperature in order to give a product of formula (VI) as defined above.

The product of formula (VII) thus obtained is subjected to the action of an amine of formula (VIII) as defined above, in particular in dichloromethane or a dichloromethane/THF mixture or dimethylformamide at room temperature, in the presence of an organic base such as triethylamine, diisopropylethylamine or N-methylmorpholine, in order to give a product of formula (I′) as defined above.

The reaction for deprotection of the carbamate functional group of the compound of formula (A) in order to obtain a product of formula (IX) may be carried out using for example an acidic agent such as pure trifluoroacetic acid at a temperature close to 0° C. or a mixture of this acid with a suitable solvent such as methylene chloride at about 0° C. or alternatively using hydrochloric acid in solution in ether or dioxane at a temperature between 0° C. and room temperature.

The product of formula (IX) is subjected to reductive amination conditions in the presence of the aldehyde or the ketone of formula (X) in order to give a product of formula (I₂) as defined above for example in sodium borocyanide or sodium triacetoxyborohydride in a solvent such as methanol, tetrahydrofuran (THF) or a mixture thereof in a pH medium between 4 and 7.

Depending on the values of R₁′, R2, R3, R4 and R5, and RZ′, the products of formulae (I) and (I₂) as defined above may therefore constitute products of formula (I) as defined above or may be converted to products of formula (I) by the customary methods known to a person skilled in the art and for example by subjecting to one or more of the reactions a) to f) indicated above.

Moreover, it may be noted that such conversion reactions a) to f) of substituents to other substituents may also be carried out on the starting materials and on the intermediates as defined above before continuing the synthesis according to the reactions indicated in the above processes.

The various reactive functional groups which certain compounds of the reactions defined above may carry may, if necessary, be protected: these are for example hydroxyl, acyl or alternatively amino and monoalkylamino radicals which may be protected with appropriate protecting groups.

The following nonexhaustive list of examples of protection of reactive functional groups may be mentioned:
the hydroxyl groups may be protected for example with alkyl radicals such as tert-butyl, trimethylsilyl, tert-butyldimethylsilyl, methoxymethyl, tetrahydropyranyl, benzyl or acetyl,
the amino groups may be protected for example with acetyl, trityl, benzyl, tert-butoxycarbonyl, benzyloxy-carbonyl and phthalimido radicals or other radicals known in peptide chemistry: the amine functional groups may in particular be protected with a group such as Boc or CH2-phenyl and may then be released under the customary conditions known to a person skilled in the art.

The reactions to which the products of formula (I′) as defined above may be subjected, if desired or if necessary, may be carried out, for example, as indicated below.

The saponification reactions may be carried out according to the customary methods known to a person skilled in the art, such as for example in a solvent such as methanol or ethanol, dioxane or dimethoxyethane, in the presence of sodium hydroxide or potassium hydroxide.

The reduction or oxidation reactions may be carried out according to the customary methods known to a person skilled in the art such as for example in a solvent such as ethyl ether or tetrahydrofuran, in the presence of sodium borohydride or lithium aluminum hydride, or for example in a solvent such as acetone or tetrahydrofuran in the presence of potassium permanganate or pyridinium chlorochromate.

a) The optional alkylthio groups of the products described above may be, if desired, converted to the corresponding sulfoxide or sulfone functional groups under the customary conditions known to a person skilled in the art such as for example with peracids such as for example peracetic acid or meta-chloroperbenzoic acid or alternatively with oxone, sodium periodate in a solvent such as for example methylene chloride or dioxane at room temperature.

The production of the sulfoxide functional group may be promoted by an equimolar mixture of the product containing an alkylthio group and the reagent such as in particular a peracid.

The production of the sulfone functional group may be promoted by a mixture of the product containing an alkylthio group with an excess reagent such as in particular a peracid.

b) The optional alkoxy functional groups such as in particular methoxy of the products described above may be, if desired, converted to a hydroxyl functional group under the customary conditions known to a person skilled in the art, for example with boron tribromide in a solvent such as for example methylene chloride, with pyridine hydrobromide or hydrochloride or alternatively with hydrobromic or hydrochloric acid in water or trifluoroacetic acid under reflux.

c) The optional alcohol functional groups of the products described above may be, if desired, converted to an aldehyde or ketone functional group by oxidation under the customary conditions known to a person skilled in the art, such as for example by the action of manganese oxide in order to obtain the aldehydes or by the action of potassium permanganate or pyridinium chlorochromate in order to obtain the ketones.

d) The removal of the protecting groups such as for example those indicated above may be carried out under the customary conditions known to a person skilled in the art, in particular by acid hydrolysis carried out with an acid such as hydrochloric, benzenesulfonic or para-toluenesulfonic, formic or trifluoroacetic acid or alternatively by catalytic hydrogenation.

The phthalimido group may in particular be removed with hydrazine.

A list of the various protecting groups which can be used may be found for example in patent BF 2 499 995.

e) The products described above may, if desired, be subjected to salification reactions for example with an inorganic or organic acid according to the customary methods known to a person skilled in the art.

f) The optional optically active forms of the products described above may be prepared by resolution of the racemates according to the customary methods known to a person skilled in the art.

Illustrations of such reactions defined above are given in the preparation of the examples described below.

The starting materials of formulae (II), (III), (V), (VIII) and (IX) may be known, may be commercially obtained or may be prepared according to the customary methods known to a person skilled in the art, in particular from commercial products, for example by subjecting them to one or more reactions known to a person skilled in the art such as for example the reactions described above in a) to f).

The products of formula (II) which are therefore pyrimidine derivatives and the products of formula (III) which are aniline derivatives may be commercial products such as for example dichloropyrimidine, trichloropyrimidine, 4-fluoroaniline, 3,4-difluoro-aniline, 4-fluoro-3-chloroaniline, or aniline.

The anilines of formula (III) may be in particular commercial anilines such as for example the following trihalogenated anilines:
3,4,5-trifluoroaniline
2,3,4-trifluoroaniline
2-chloro-4,6-difluoroaniline
2,4,5,-trifluoroaniline
3-chloro-2,4-difluoroaniline
2,4-dichloro-5-fluoroaniline.

The aniline of formula (V) is commercially available.

The amines of formula (VIII) may also be commercially available, such as for example methyl-(1-methylpiperidin-4-yl)amine.

The amines of formula (VIII) used in stage 4 of examples 1, 4 to 8, 11, 12, 14 and 18 to 20 are commercially available.

The preparations of the noncommercial amines of formula (VIII) may be carried out according to methods known to a person skilled in the art and in particular by the three procedures 1, 2 and 3 indicated below in the experimental part.

It may be stated that in order to obtain the products of formula (I) as defined above in which R1, R2, R3, R4, R5 and A have the meanings indicated above, and ring (Y) is such that Y represents NR7 and contains a carbon bridge consisting of 1 to 3 carbons, it is possible to use, as starting materials, bicyclic amines which may be obtained from commercial compounds such as tropinone, pseudo-pelletrivine according to the references below:
Tetrahedron 2002, 58, 5669-5674
J. Org. Chem. 1996, 61, 3849-3862
J. Med. Chem. 1993, 36, 3703-3720
J. Chem. Soc. Perkin Trans1 1991, 1375-1381
J. Med. Chem. 1994, 37, 2831-2840

By way of examples, the following compounds may be mentioned:

### N,9-dimethyl-9-azabicyclo[3.3.1]nona-3-amine

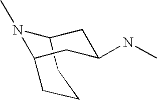

### N,6-dimethyl-6-azabicyclo[3.2.1]octan-3-amine

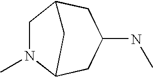

### N,3-dimethyl-3-azabicyclo[3.2.1]octan-8-amine

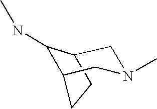

### N,3-dimethyl-3-azabicyclo[3.3.1]nonan-9-amine

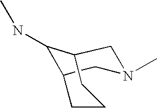

Examples of aldehydes or ketones of formula (X) are given in the experimental part by way of nonlimiting examples.

The present invention also relates to the process according to scheme 1 below for preparing products of formula (I) as defined above:

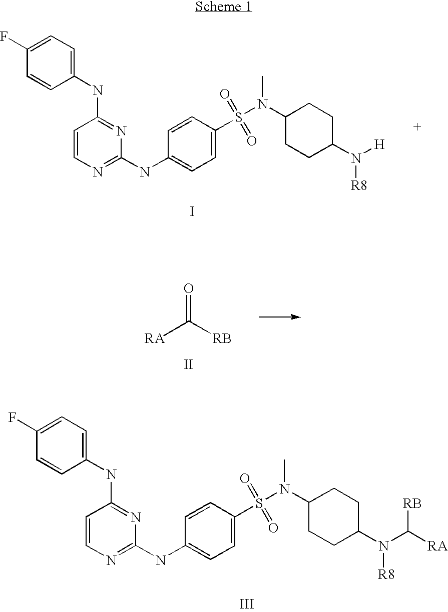

In such a scheme 1, the radical NR8-CH(RA)(RB) represents certain values of NR8R9 as defined above with R8 as defined above and R9 represents —CH(RA)(RB), that is to say, as defined for R9, a linear or branched alkyl radical optionally substituted with one or more radicals chosen from halogen atoms and the radicals hydroxyl, alkoxy, NH2, NHalkyl, N(alkyl)2, alkylthio, phenyl and saturated or unsaturated heterocycles, phenyl and heterocycle which are themselves optionally substituted as indicated above.

In particular, RA may represent a hydrogen atom or CH3, and RB may represent (CH2)n-A with A representing a heterocycle or phenyl radical optionally substituted as defined above and n representing an integer from 0 to 5.

The steps of the synthesis process of scheme 1 above may be carried out according to the customary methods known to a person skilled in the art and in particular as described below for the preparation of Examples 66 to 85.

The present invention also relates to the method according to scheme 2 below, for preparing products of formula (I) as defined above:

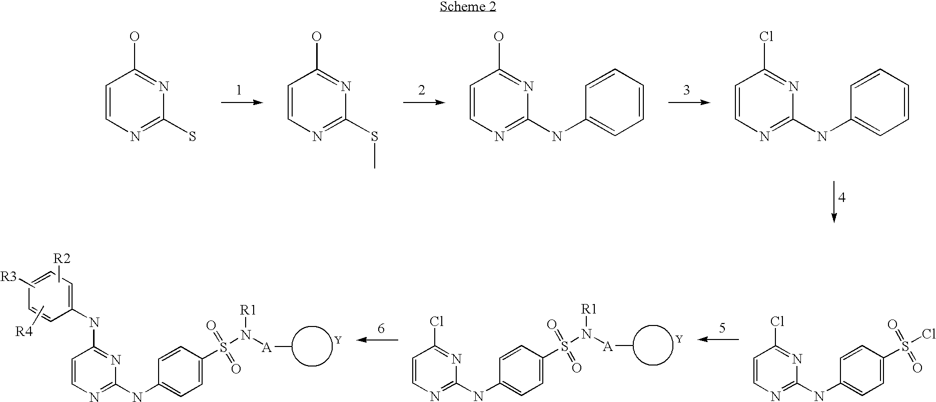

In such a scheme 2, R1, R2, R3, R4, A and ring (Y) have the meanings indicated above for the products of formula (I).

The steps of the process of synthesis of scheme 2 above may be carried out according to the customary methods known to a person skilled in the art and in particular as illustrated below for the preparation of examples 152 and 156.

The experimental part below gives nonlimiting examples of preparation of products of formula (I) according to the present invention and also examples of nonlimiting starting materials used in these preparations.

The subject of the present invention is finally, as novel industrial products, certain compounds of formulae (VII) and (IX).

The products of formula (I) as defined above and their addition salts with acids have advantageous pharmacological properties.

The compounds of the present invention may therefore inhibit the activity of kinases, in particular IKK1 and IKK2 with an IC50 of less than 10 μM.

The compounds of the present invention may thus inhibit the activation of NF-KB, and the production of cytokines with IC50 values of less than 10 μM.

The compounds of the present invention may thus inhibit the proliferation of a large panel of tumor cells with IC50 values of less than 10 μM.

The compounds of formula (I) may therefore have a medicament activity, in particular as IKK1 and IKK2 inhibitors and may be used in the prevention or treatment of diseases in which the inhibition of IKK1 or IKK2 is beneficial. For example, the prevention or treatment of diseases such as inflammatory diseases or diseases with an inflammatory component such as for example inflammatory arthritis including rheumatoid arthritis, spondylitic osteoarthritis, Reiter's syndrome, psoriatic arthritis, bone resorption diseases; multiple sclerosis, inflammatory bowel diseases including Crohn's disease; asthma, chronic pulmonary obstruction, emphysema, rhinitis, acquired myasthenia, graves' disease, graft rejection, psoriasis, dermatitis, allergic disorders, immune system diseases, cachexia, severe acute respiratory syndrome, septic shock, cardiac insufficiency, myocardial infarction, atherosclerosis, reperfusion lesions, AIDS, cancer and disorders characterized by insulin resistance such as diabetes, hyperglycemia, hyperinsulinemia, dyslipidemia, obesity, polycystic ovary diseases, hypertension, cardiovascular disorders, syndrome X, autoimmune diseases such as in particular systemic lupus, lupus erythematosus, glomerulonephritis induced by immune system deficiencies, insulin-dependent autoimmune diabetes, retinitis pigmentosa, aspirin-sensitive rhinosinusitis.

The products of formula (I) according to the present invention as modulators of apoptosis may be useful in the treatment of various human diseases including aberrations in apoptosis such as cancer: such as in particular, but without limitation, follicular lymphomas, carcinomas with p53 mutations, hormone-dependent breast tumors, prostate and ovarian tumors, and precancerous lesions such as familial adenomatous polyposis, viral infections (such as in particular, but without limitation, those caused by the Herpes virus, the poxvirus, the Epstein-Barr virus, Sindbis virus and adenovirus), myelodysplastic syndromes, ischemic disorders associated with myocardial infarction, cerebral congestion, arrhythmia, atherosclerosis, hepatic disorders induced by toxins or alcohol, hematological disorders such as in particular, but without limitation, chronic anemia and a plastic anemia, degenerative diseases of the musculoskeletal system such as in particular, but without limitation, osteoporosis, cystic fibrosis, kidney diseases and cancer.

It is therefore evident that the compounds according to the invention have anticancer activity and activity in the treatment of other proliferative diseases such as psoriasis, restenosis, atherosclerosis, AIDS for example, and in diseases caused by the proliferation of the vascular smooth muscle cells, angiogenesis and in rheumatoid arthritis, neurofibromatosis, atherosclerosis, pulmonary fibrosis, restenosis following angioplasty or vascular surgery, the formation of hypertrophic scars, angiogenesis and endotoxic shock.

These medicaments find their use in therapy, in particular in the treatment or prevention of diseases caused or exacerbated by the proliferation of cells and in particular of tumor cells.

As inhibitor of tumor cell proliferation, these compounds are useful in the prevention and treatment of leukemia, both primary and metastatic solid tumors, carcinomas and cancers, in particular: breast cancer, lung cancer, cancer of the small intestine, cancer of the colon and rectum, cancer of the respiratory tracts, of the oropharynx and of the hypopharynx, cancer of the esophagus, liver cancer, stomach cancer, cancer of the bile ducts, cancer bile vesicle, pancreatic cancer, cancer of the urinary tracts including kidney, urothelium and bladder, cancers of the female genital tract including uterine, cervical and ovarian cancers, chloriocarcinoma and trophoblastoma; cancers of the male genital tract including cancer of the prostate, the seminal vesicles and the testicles, tumors of the germ cells; cancers of the endocrine glands including cancer of the thyroid, the pituitary gland and the adrenal glands; skin cancer including hemangiomas, melanomas, sarcomas, including Kaposi's sarcoma; tumors of the brain, the nerves, the eyes and the meninges, including astrocytomas, gliomas, glioblastomas, retino-blastomas, neurinomas, neuroblastomas, schwannomas, meningiomas; hematopoietic malignant tumors; leukemias such as acute lymphoid leukemia, acute myeloid leukemia, chronic myeloid leukemia, chronic lymphoid leukemia, chloromas, plasmocytomas, T or B cell leukemia, Hodgkin or nonHodgkin lymphomas, myelomas, various malignant hemopathies.

The subject of the present invention is in particular the combinations defined as follows.

According to the present invention, the compound(s) of formula (I) may be administered in combination with one or more anticancer active ingredients, in particular antitumor compounds such as alkylating agents such as alkyl sulfonates (busulfan), dacarbazine, procarbazine, nitrogen mustards (chlormethine, melphalan, chlor-ambucil), cyclophosphamide, ifosfamide; nitrosoureas such as carmustine, lomustine, semustine, streptozocin; antineoplastic alkaloids such as vincristine, vinblastine; taxanes such as paclitaxel or taxotere; antineoplastic antibiotics such as actinomycin; intercalating agents, antineoplastic antimetabolites, antagonists of folates, methotrexate; inhibitors of the synthesis of purines; purine analogues such as mercaptopurine, 6-thioguanine; inhibitors of the synthesis of pyrimidines, aromatase inhibitors, capecitabine, pyrimidine analogues such as fluorouracil, gemcitabine, cytarabine and cytosine arabinoside; brequinar; inhibitors of topoisomerases such as camptothecin or etoposide; anticancer hormonal agonists and antagonists including tamoxifen; kinase inhibitors, imatinib; growth factor inhibitors; anti-inflammatory agents such as pentosan polysulfate, corticosteroids, prednisone, dexamethasone; antitopo-isomerases such as etoposide, anthracyclines including doxorubicin, bleomycin, mitomycin and methramycin; anticancer metal complexes, platinum complexes, cisplatin, carboplatin, oxaliplatin; interferon-alpha, triphenylthiophosphoramide, altretamine; antiangiogenic agents; thalidomide; immunotherapy adjuvants; vaccines.

According to the present invention, the compounds of formula (I) may also be administered in combination with one or more other active ingredients useful in one of the pathologies indicated above, for example an antiemetic, antipain, anti-inflammatory and anticachexia agent.

The subject of the present invention is thus, as medicaments, the products of formula (I) as defined above and the addition salts with pharmaceutically acceptable inorganic and organic acids of said products of formula (I).

The subject of the present invention is in particular, as medicaments, the products of formula (I) as defined in any one of the preceding claims whose names are as follows:
2-{4-[4-(4-fluorophenylamino)pyrimidin-2-ylamino]-benzenesulfonylamino}-N-(tetrahydropyran-4-yl)acetamide
4-[4-(4-fluorophenylamino)pyrimidin-2-ylamino]-N-methyl-N-(1-pyridin-2-ylmethylpiperidin-4-yl)benzene-sulfonamide
N-(2-dimethylaminoethyl)-4-[4-(4-fluorophenylamino)-pyrimidin-2-ylamino]-N-(1-methylpiperidin-4-yl)benzene-sulfonamide
4-[4-(4-fluorophenylamino)pyrimidin-2-ylamino]-N-(2-hydroxyethyl)-N-(1-methylpiperidin-4-yl)benzenesulfon-amide
4-[4-(4-fluorophenylamino)pyrimidin-2-ylamino]-N-methyl-N-(1-pyridyl-3-ylmethylpiperidin-4-yl)benzene-sulfonamide
4-[4-(4-fluorophenylamino)pyrimidin-2-ylamino]-N-piperidin-4-yl-N-(2-pyrrolidin-1-ylethyl)benzene-sulfonamide hydrochloride
N-(2-aminoethyl)-4-[4-(3-chloro-4-fluorophenylamino)-pyrimidin-2-ylamino]-N-(piperidin-4-yl)benzenesulfonamide hydrochloride
   and the addition salts with pharmaceutically acceptable inorganic and organic acids of said products of formula (I).

The subject of the present invention is in particular, as medicaments, the products of formula (I) as defined above whose names are as follows:
2-{4-[4-(4-fluorophenylamino)pyrimidin-2-ylamino]-benzenesulfonylamino}-N-(tetrahydropyran-4-yl)acetamide
4-[4-(4-fluorophenylamino)pyrimidin-2-ylamino]-N-methyl-N-(1-pyridin-2-ylmethylpiperidin-4-yl)benzenesulfonamide
N-(2-dimethylaminoethyl)-4-[4-(4-fluorophenylamino)-pyrimidin-2-ylamino]-N-(1-methylpiperidin-4-yl)benzene-sulfonamide
4-[4-(4-fluorophenylamino)pyrimidin-2-ylamino]-N-(2-hydroxyethyl)-N-(1-methylpiperidin-4-yl)benzene-sulfonamide
4-[4-(4-fluorophenylamino)pyrimidin-2-ylamino]-N-methyl-N-(1-pyridyl-3-ylmethylpiperidin-4-yl)benzene-sulfonamide
   and the addition salts with pharmaceutically acceptable inorganic and organic acids of said products of formula (I).

The subject of the present invention is also the pharmaceutical compositions containing, as active ingredient, at least one of the products of formula (I) as defined above or a pharmaceutically acceptable salt of this product or a prodrug of this product and a pharmaceutically acceptable carrier.

The subject of the present invention is particularly the use of the products of formula (I) as defined above or of pharmaceutically acceptable salts of these products for the preparation of a medicament intended for the treatment or prevention of a disease by inhibiting the activity of the protein kinase IKK.

The subject of the present invention is thus the use, as defined above, in which the protein kinase is in a mammal.

The subject of the present invention is thus the use of a product of formula (I) as defined above for the preparation of a medicament intended for the treatment or prevention of a disease chosen from the diseases indicated above.

The subject of the present invention is in particular the use of a product of formula (I) as defined above for the preparation of a medicament intended for the treatment or prevention of a disease chosen from the following group: inflammatory diseases, diabetes and cancer.

The subject of the present invention is in particular the use of a product of formula (I) as defined above for the preparation of a medicament intended for the treatment or prevention of inflammatory diseases.

The subject of the present invention is in particular the use of a product of formula (I) as defined above for the preparation of a medicament intended for the treatment or prevention of diabetes.

The subject of the present invention is in particular the use of a product of formula (I) as defined above for the preparation of a medicament intended for the treatment of cancer.

The subject of the present invention is in particular the use of a product of formula (I) as defined above intended for the treatment of solid or liquid tumors.

The subject of the present invention is in particular the use of a product of formula (I) as defined above intended for the treatment of cancers resistant to cytotoxic agents.

The subject of the present invention is in particular the use of a product of formula (I) as defined above for the preparation of medicaments intended for cancer chemotherapy.

The subject of the present invention is in particular the use of a product of formula (I) as defined above for the preparation of medicaments intended for cancer chemotherapy alone or in combination or in the form of a combination as defined above.

The subject of the present invention is in particular the use of a product of formula (I) as defined above as IKK inhibitors.

The present invention relates most particularly to the products of formula (I) as defined above which constitute examples 1 to 169 of the present invention.

The following examples illustrate the invention without however limiting it.

The products of tables I, II, III and IV below also form part of the present invention and may be prepared according to the methods described in the present invention and, where appropriate, also by methods known to persons skilled in the art.

TABLE I

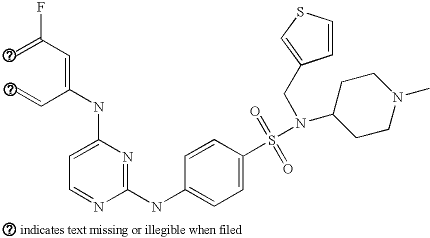

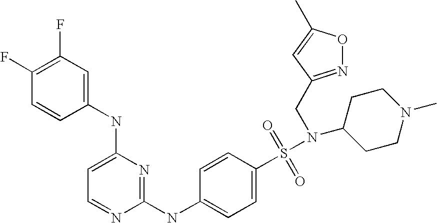

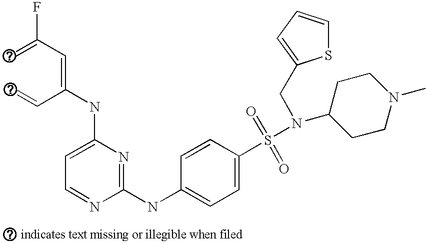

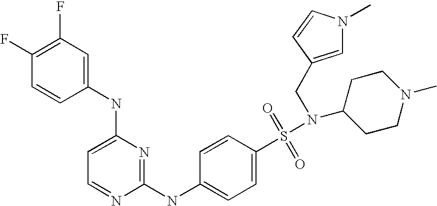

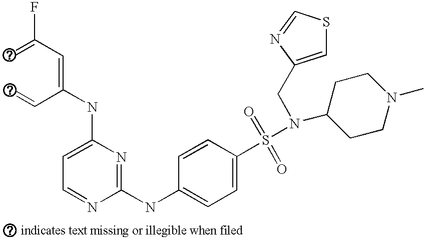

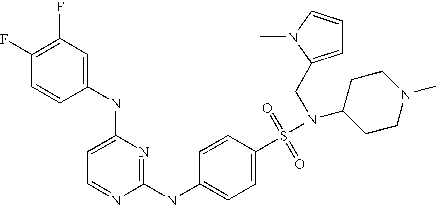

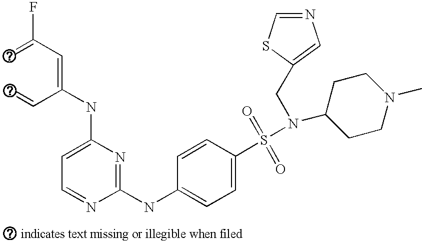

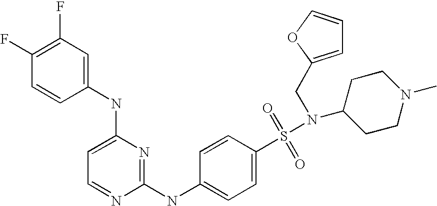

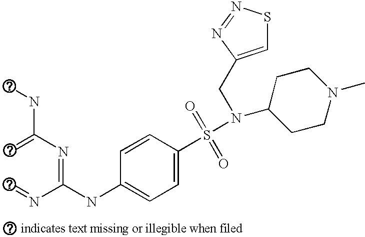

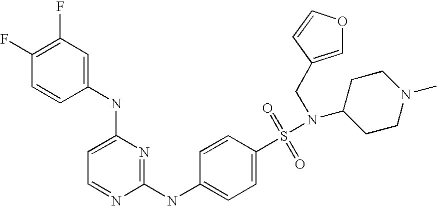

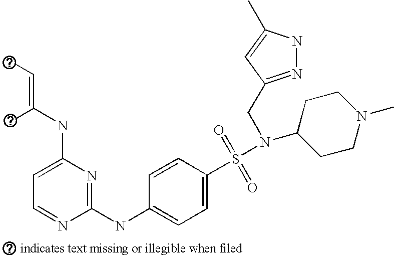

TABLE II

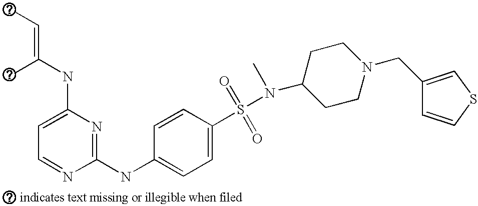

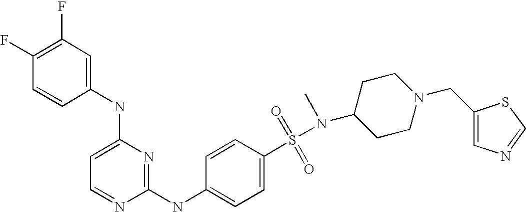

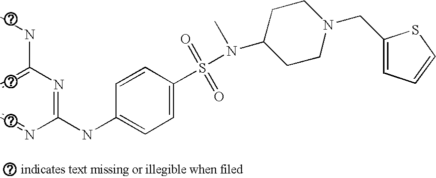

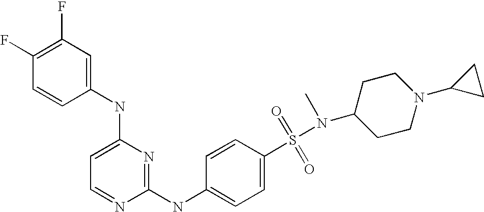

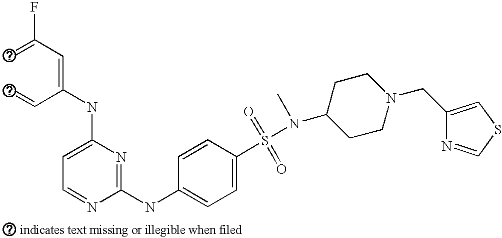

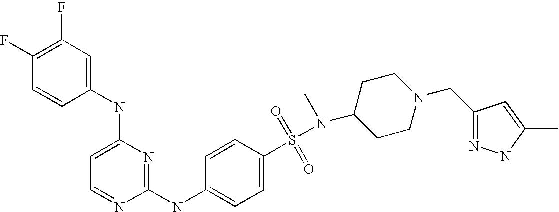

TABLE III

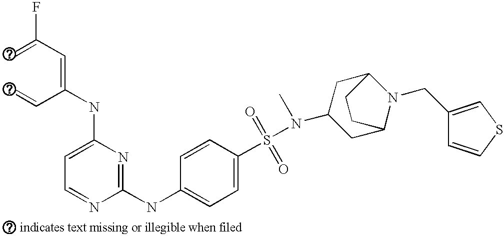

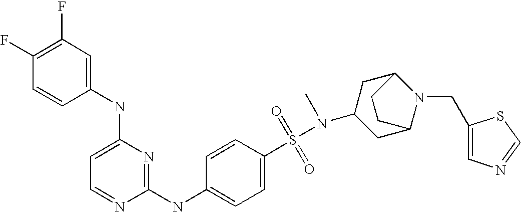

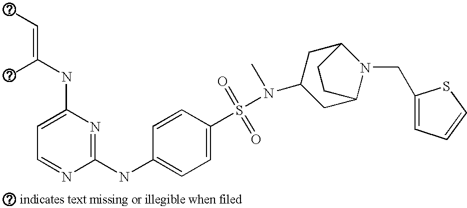

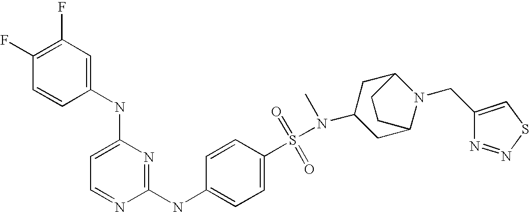

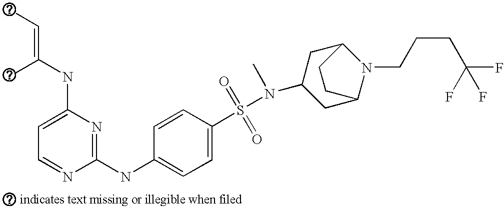

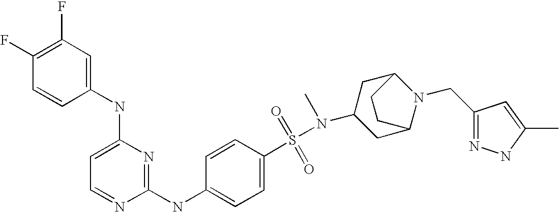

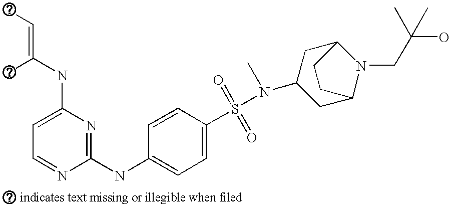

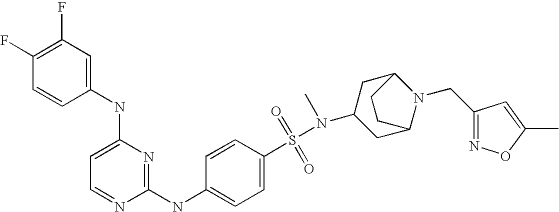

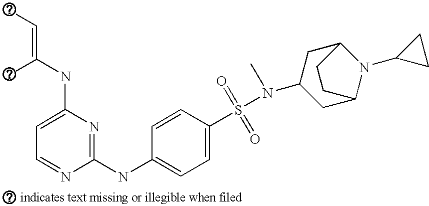

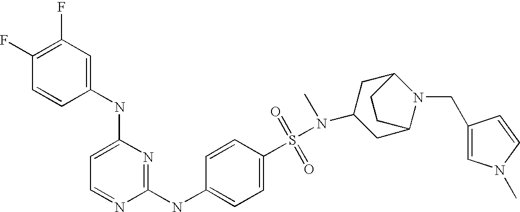

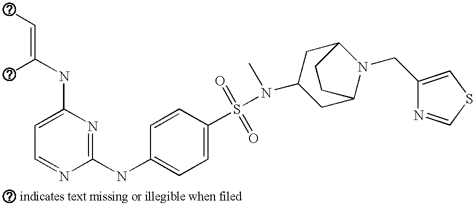

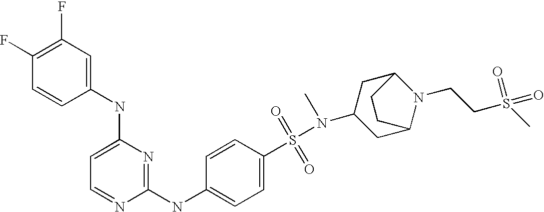

TABLE IV

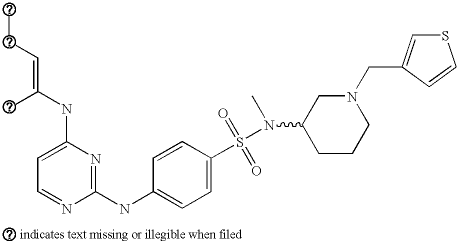

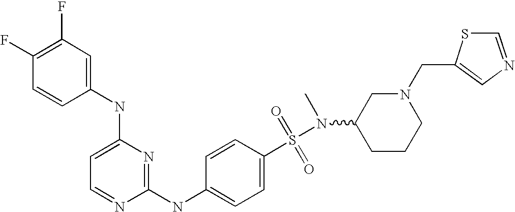

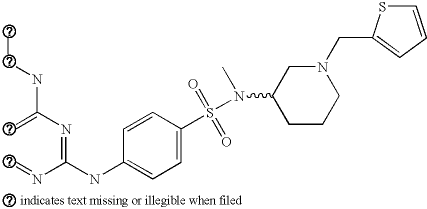

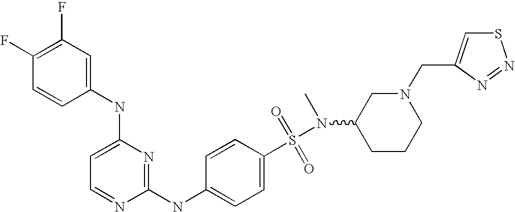

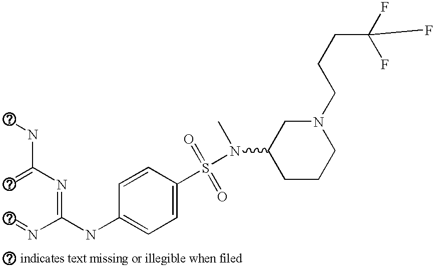

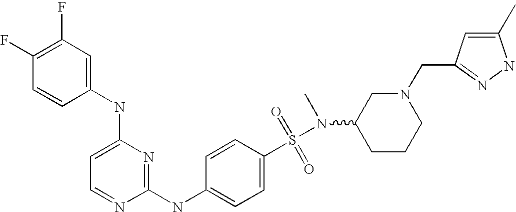

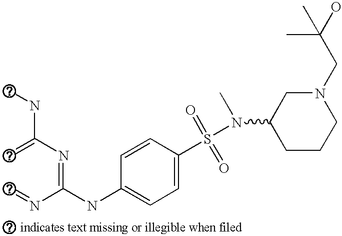

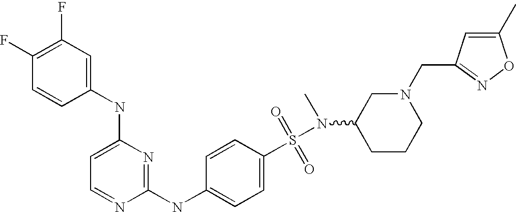

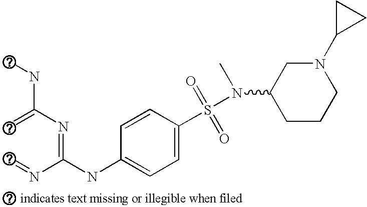

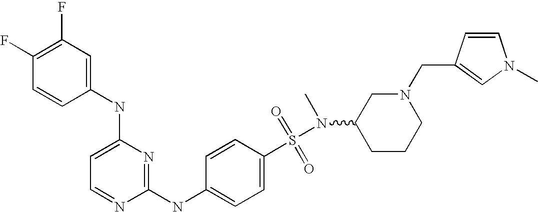

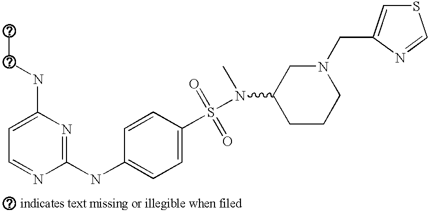

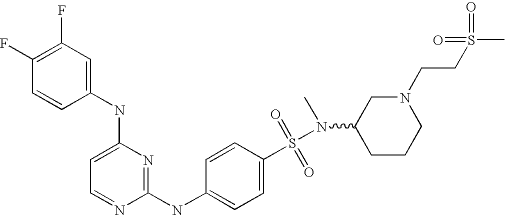

The following examples illustrate the invention without however limiting it.

### EXPERIMENTAL PART

The noncommercial amines used at stage 4 of the preparation of the examples of the present invention may be prepared according to procedures 1, 2 and 3 described below.

### Procedure 1:

Preparation of the Amine Used at Stage 4 of Example 3: (1-benzylpiperidin-4-yl)methylamine hydrochloride:

5 g of 1-benzylpiperidin-4-one are dissolved in 60 ml of THF. 13.3 ml of a 2M solution of methylamine in THF are added followed by 5.6 g of sodium triacetoxyborohydride. The reaction medium is left at room temperature overnight. 10 ml of methanol are added to the reaction medium and then the medium is heated at 70° C. for 1 h 30 min. After concentrating to dryness and taking up in a sodium hydroxide solution, the medium is extracted with dichloromethane and the chlorinated phase is dried over Na2SO4. 6 g of a product are obtained, which product is dissolved in 100 ml of dichloromethane. To this dichloromethane solution are added 3.5 g of Boc2O which causes emission of CO2. After concentrating to dryness, the crude reaction product is chromatographed on a silca column in order to give 4.9 g of (1-benzylpiperidin-4-yl)methylcarbamic acid tert-butyl ester. 1.22 g of (1-benzylpiperidin-4-yl)methylcarbamic acid tert-butyl ester are kept stirring in 40 ml of 2N hydrochloric ether. After one night, the reaction medium is filtered in order to obtain 0.9 g of expected product in hydrochloride form.

Preparation of the Amine Used at Stage 4 of Example 15: (1-benzylazepan-4-yl)methylamine hydrochloride:

The procedure is carried out as in procedure 1 starting with 2 g of benzylazepan-4-one and 12.9 ml of a 2M solution of methylamine in THF. 1.9 g of expected product are thus obtained.

Preparation of the Amine Used at Stage 4 of Example 21: methyl(1-methylazepan-4-yl)amine hydrochloride (racemate):

The procedure is carried out as in procedure 1 starting with 2 g of 1-methylazepan-4-one and 1.8 ml of a 2M solution of methylamine in THF. 1.15 g of expected product are thus obtained.

### Procedure 2:

Preparation of the Amine Used at Stage 4 of Example 9: methyl(1-pyridin-2-ylmethylpiperidin-4-yl)amine hydrochloride:

500 mg of pyridine-2-carbaldehyde is dissolved in 10 ml of THF. 1 g of methylpiperidin-4-ylcarbamic acid tert-butyl ester is added followed by 1 g of sodium triacetoxyborohydride. The reaction medium is kept stirring at room temperature overnight. 10 ml of methanol are added to the reaction medium and then the medium is heated at 70° C. for 1 h 30 min. After concentrating to dryness and taking up in a sodium hydroxide solution, the medium is extracted with dichloromethane and the chlorinated phase is dried over Na2SO4. 1 g of methyl(1-pyridin-2-ylmethylpiperidin-4-yl)carbamic acid tert-butyl ester is obtained, which is dissolved in 40 ml of a solution of hydrochloric ether overnight. The reaction medium is filtered in order to obtain 900 mg of expected product in hydrochloride form.

Preparation of the Amine Used at Stage 4 of Example 10: 1-(ethylpiperidin-4-yl)methylamine hydrochloride:

The procedure is carried out as in example 1 of procedure 2 starting with 1 g of methylpiperidin-4-ylcarbamic acid tert-butyl ester and 210 mg of acetaldehyde. 680 mg of expected product are thus obtained.

Preparation of the Amine Used at Stage 4 of Example 22: methyl(1-pyridin-3-ylmethylpiperidin-4-yl)amine hydrochloride:

The procedure is carried out as in example 1 of procedure 2 starting with 1 g of methylpiperidin-4-ylcarbamic acid tert-butyl ester and 500 mg of pyridine-3-carbaldehyde. 880 mg of expected product are thus obtained.

Preparation of the Amine Used at Stage 4 of Example 24: methyl-(1-pyridin-4-ylmethylpiperidin-4-yl)amine hydrochloride:

The procedure is carried out as in example 1 of procedure 2 starting with 1 g of methylpiperidin-4-ylcarbamic acid tert-butyl ester and 500 mg of pyridine-4-carbaldehyde. 850 mg of expected product are thus obtained.

Preparation of the Amine Used at Stage 4 of Example 23: methyl(1-thiazol-2-ylmethylpiperidin-4-yl)amine hydrochloride:

The procedure is carried out as in example 1 of procedure 2 starting with 1 g of methylpiperidin-4-ylcarbamic acid tert-butyl ester and 532 mg of thiazole-2-carbaldehyde. 940 mg of expected product are thus obtained.

Preparation of the Amine Used at Stage 4 of Example 16: methyl(1-thiophen-3-ylmethylpiperidin-4-yl)amine hydrochloride:

The procedure is carried out as in example 1 of procedure 2 starting with 1 g of methylpiperidin-4-ylcarbamic acid tert-butyl ester and 527 mg of thiophene-3-carbaldehyde. 820 mg of expected product are thus obtained.

Preparation of the Amine Used at Stage 4 of Example 13: methyl(1-thiophen-2-ylmethylpiperidin-4-yl)amine hydrochloride:

The procedure is carried out as in example 1 of procedure 2 starting with 1 g of methylpiperidin-4-ylcarbamic acid tert-butyl ester and 527 mg of thiophene-2-carbaldehyde. 760 mg of expected product are thus obtained.

Preparation of the Amine Used at Stage 4 of Example 26: methyl(1-pyrazin-2-ylmethylpiperidin-4-yl)amine hydrochloride:

The procedure is carried out as in example 1 of procedure 2 starting with 1 g of methylpiperidin-4-ylcarbamic acid tert-butyl ester and 508 mg of pyrazine-2-carbaldehyde. 795 mg of expected product are thus obtained.

Preparation of the Amine Used at Stage 4 of Example 27: (1-furan-2-ylmethylpiperidin-4-yl)methylamine hydrochloride:

The procedure is carried out as in example 1 of procedure 2 starting with 1 g of methylpiperidin-4-ylcarbamic acid tert-butyl ester and 452 mg of furan-2-carbaldehyde. 760 mg of expected product are thus obtained.

Preparation of the Amine Used at Stage 4 of Example 28: [1-(3H-imidazol-4-ylmethyl)piperidin-4-yl]methylamine hydrochloride:

The procedure is carried out as in example 1 of procedure 2 starting with 1 g of methylpiperidin-4-ylcarbamic acid tert-butyl ester and 452 mg of 3H-imidazole-4-carbaldehyde. 780 mg of expected product are thus obtained.

### Procedure 3

Preparation of the Amine Used at Stage 4 of Example 25: 2-amino-N-methyl-N-(1-methylpiperidin-4-yl)acetamide hydrochloride:

To a solution containing 3.54 g of chloro-3,5-dimethoxytriazine and 3.58 g of tert-butoxycarbonyl-aminoacetic acid in 20 ml of dichloromethane are added dropwise to 2.25 ml of N-methylmorpholine while maintaining the reaction temperature between −5 and 0° C. The stirring is maintained for 4 hours and complete consumption of chloro-3,5-dimethoxytriazine is observed. A mixture containing 2.9 ml of methyl(1-methylpiperidin-4-yl)amine and 2.25 ml of N-methylmorpholine in 10 ml of dichloromethane is added, while maintaining the reaction temperature between −5 and 0° C. The reaction medium is stirred at 0° C. for 2 hours and at room temperature overnight. The solvent is evaporated and the residue is taken up in 70 ml of ethyl acetate. The suspension is successively washed with H2O (30 ml), 30 ml of a 10% citric acid solution, H2O (30 ml), 30 ml of a saturated NaHCO3 solution and H2O (30 ml). After drying over mgSO2 and chromatography on silica (eluent 10% methanol in dichloromethane), 4 g of a product are obtained which are kept stirring in 100 ml of ether/HCl. After one night, the reaction medium is filtered in order to obtain 2.5 g of expected product.

Preparation of the Amine Used at Stage 4 of Example 2: 2-amino-N-(tetrahydropyran-4-yl)acetamide hydrochloride:

The procedure is carried out as in example 1 of procedure 2 starting with 1 g of tert-butoxycarbonylaminoacetic acid and 578 mg of tetrahydropyran-4-ylamine. 700 mg of expected product are thus obtained.

### Example 1

### 4-[4-(4-Fluorophenylamino)pyrimidin-2-ylamino]-N-methyl-N-(1-methylpiperidin-4-yl)benzenesulfonamide

### Stage 1: (2-Chloropyrimidin-4-yl)(4-fluorophenyl)amine

To a stirred mixture containing 15 g of dichloro-pyrimidine in 200 ml of n-butanol are added 10 ml of 4-fluoroaniline and then 18 ml of diisopropylethylamine. The reaction mixture is heated, with stirring, under reflux for 2 hours. The reaction medium is cooled and concentrated to dryness. A K2CO3 solution is added to the residue and the medium is extracted 3 times with ethyl acetate, washing with a saturated NaCl solution and drying over Na2SO4, the crude reaction product is purified by chromatography on a silica column (CH2CL2 and then 30% ethyl acetate in CH2Cl2). During the concentration, 11 g of expected compound crystallize.

(MH+=224), PF=172-174° C.

### Stage 2: N-4-(4-Fluorophenyl)-N-2-phenylpyrimidine-2,4-diamine

10.5 g of (2-chloropyrimidin-4-yl)(4-fluorophenyl)amine in solution in 300 ml of n-butanol are heated at 140° C. under reflux in the presence of 4.3 ml of aniline overnight. The reaction medium is cooled. The suspension obtained is filtered. The crystals are taken up in ethyl acetate and washed with a 10% K2CO3 solution and then with a saturated NaCl solution. After drying over Na2SO4, the organic phase is concentrated under vacuum. The crude reaction product is purified by chromatography on a silica column (10% THF, 5% MeOH, 85% CH2CL2). The expected N-4-(4-fluorophenyl)-N-2-phenylpyrimidine-2,4-diamine crystallizes during the concentration and 10.5 g of the product are obtained by filtration. MH+=281, m.p.=161° C.

### Stage 3: 4-[4-(4-Fluorophenylamino)pyrimidin-2-ylamino]benzenesulfonyl chloride hydrochloride

To a three-necked flask under a nitrogen stream containing chlorosulfonic acid at 0° C. are added in small portions 7.5 g of N-4-(4-fluorophenyl)-N-2-phenylpyrimidine-2,4-diamine while maintaining the temperature around 0° C. The reaction medium is left at room temperature for 18 h. The mixture is poured dropwise, with care, over ice. The precipitate obtained is filtered and washed with distilled water. After dissolving the solid in 1 l of ethyl acetate, drying over Na2SO4 and concentrating under vacuum, a whitish oil is obtained. This oil precipitates after dispersion in 200 ml of ether. 10.5 g of 4-[4-(4-fluorophenyl-amino)pyrimidin-2-ylamino]benzenesulfonyl chloride hydrochloride are obtained by filtration of the ethereal suspension. MH+=360, m.p. poorly defined.

1) Bioorg. Med. Chem. 2003, 13, 2961-2966

### Stage 4: 4-[4-(4-Fluorophenylamino)pyrimidin-2-ylamino]-N-methyl-N-(1-methylpiperidin-4-yl)benzenesulfonamide

To a solution of 400 mg of 4-[4-(4-fluorophenylamino)-pyrimidin-2-ylamino]benzenesulfonyl chloride hydro-chloride in 30 ml of dichloromethane is added 0.16 ml of methyl(1-methylpiperidin-4-yl)amine (commercial product) followed by 0.7 ml of diisopropylethylamine. The reaction mixture is kept stirring at room temperature for 18 hours. The reaction medium is concentrated to dryness and taken up in a 10% K2CO3 solution. After extracting with ethyl acetate, the organic phase is washed with a saturated NaCl solution and then dried over Na2SO4. The crude reaction product is purified by chromatography on a silica column (CH2CL2 followed by 10% methanol in CH2Cl2);

210 mg of expected compound are obtained.

MH+=471.2; melting point 205-210° C. (Isopropyl ether-dichloromethane)

1H (200 MHz (CD₃) 2SO d6, in ppm): 1.20 (d, 2H); 1.58 (m, 2H); 1.90 (t, 2H); 2.11 (s, 3H); 2.65 (s, 3H); 2.73 (d, 2H); 3.60 (m, 1H); 6.28 (d, 1H); 7.17 (t, 2H); 7.56-7.84 (unresolved complex, 4H); 7.94 (d, 2H); 8.07 (d, 1H); 9.48 (broad s, 1H); 9.67 (broad s, 1H).

The preparation of the products of examples 2 to 28 below is carried out following the same process as for example 1 using at stage 4 the appropriate amines of formula (VIII) chosen from commercial or synthesized amines as indicated by way of examples in the experimental part below. Moreover, according to the values of R2, R3, R4 and R5 of the expected product, the appropriate starting materials of formulae (II) and (II) are used in stage 1. Thus:
for examples 1 to 19 and 21 to 28, for which R5 represents hydrogen, the same starting material of formula (II) is used as for example 1.
for the product of example 20, for which R5 represents a chlorine atom, another starting material of formula (II) is used which carries at the 6-position the appropriate substituent, here a chlorine atom.
for the products of examples 1 to 10 and 13 to 28 for which R2, R3, R4 represent H, H, F, the same starting material of formula (III) is used as for example 1.
for the products of examples 11 and 12, the starting materials of formula (III) are used for which R2, R3, R4 have the appropriate meanings, here R2, R3, R4 represent H, F, Cl.

Next, after stage 1, for all the examples 2 to 28, the procedure is carried out as in stages 2 and 3 of example 1, and finally the procedure is carried out as in stage 4 of example 1 by reacting the product obtained in stage 3 with the appropriate amine of formula (VIII) in order to obtain the expected product.

### Example 2

### 2-{4-[4-(4-Fluorophenylamino)pyrimidin-2-ylamino]benzenesulfonylamino}-N-(tetrahydropyran-4-yl)-acetamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 204 mg of 2-amino-N-methyl-N-(tetrahydropyran-4-yl)acetamide hydrochloride. 260 mg of expected product are thus obtained.

MH+=501; Melting point=253-254° C. (Isopropyl ether-dichloromethane)

1H (200 MHz (CD₃) 2SO d6, in ppm): 1.28 (m, 2H); 1.56 (d, 2H); 3.25 (m, 2H); 3.38 (s, 2H); 3.47-3.83 (unresolved complex, 3H); 6.27 (d, 1H); 7.18 (t, 2H); 7.52-7.82 (unresolved complex, 6H); 7.90 (d, 2H); 8.06 (d, 1H); 9.49 (broad s, 1H); 9.63 (broad s, 1H).

### Example 3

### N-(1-Benzylpiperidin-4-yl)-4-[4-(4-fluoro-phenylamino)pyrimidin-2-ylamino]-N-methylbenzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 252 mg of (1-benzylpiperidin-4-yl)methylamine hydrochloride. 259 mg of expected product are thus obtained.

MH+=547; Melting point=186-190° C. (Isopropyl ether-dichloromethane)

1H (200 MHz (CD₃) 2SO d6, in ppm): 1.23 (d, 2H); 1.57 (m, 2H); 1.94 (t, 2H); 2.66 (s, 3H); 2.76 (d, 2H); 3.40 (s, 2H); 3.62 (m, 1H); 6.29 (d, 1H); 7.07-7.38 (unresolved complex, 7H); 7.56-7.85 (unresolved complex, 7H); 7.56-7.85 (unresolved complex, 4H); 7.94 (d, 2H); 8.08 (d, 1H); 9.48 (broad s, 1H); 9.67 (broad s, 1H).

### Example 4

### N-(1-Benzylpyrrolidin-3-S-yl)-4-[4-(4-fluorophenylamino)pyrimidin-2-ylamino]-N-methylbenzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 200 mg of (1-benzyl-pyrrolidin-3-S-yl)methylamine (commercial product). 298 mg of expected product are thus obtained.

MH+=533; Melting point=154-155° C. (Isopropyl ether-dichloromethane); α_{D}=−21.4 (C=0.116, MeOH)

1H (200 MHz (CD3) 2SO d6, in ppm): 1.44 (m, 1H); 1.76 (m, 1H); 1.93-2.35 (unresolved complex, 6H); 2.56 (m, 1H); 2.66 (s, 3H); 4.46 (m, 1H); 6.30 (d, 1H); 7.19 (d, 2H); 7.55-7.80 (unresolved complex, 4H); 7.97 (d, 2H); 8.10 (d, 1H); 9.49 (broad s, 1H); 9.69 (broad s, 1H).

### Example 5

### 4-[4-(4-Fluorophenylamino)pyrimidin-2-yl-amino]-N-methyl-N-(1-methylpyrrolidin-3-yl)benzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 120 g of racemic methyl(1-methylpyrrolidin-3-yl)amine (commercial product).

215 mg of expected product are thus obtained.

MH+=457; Melting point=177-181° C. (Isopropyl ether-dichloromethane)

1H (200 MHz (CD3) 2SO d6, in ppm): 1.44 (m, 1H); 1.76 (m, 1H); 1.93-2.35 (unresolved complex, 6H); 2.56 (m, 1H); 2.66 (s, 3H); 4.46 (m, 1H); 6.30 (d, 1H); 7.19 (t, 2H); 7.55-7.80 (unresolved complex, 4H); 7.97 (d, 2H); 8.10 (d, 1H); 9.49 (broad s, 1H); 9.69 (broad s, 1H).

### Example 6

### N-(1,1-Dioxotetrahydro-1λ⁶-thiophen-3-yl)-4-[4-(4-fluorophenylamino)pyrimidin-2-ylamino]-N-methyl-benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 158 mg of racemic 1,1-dioxotetrahydro-1λ⁶-thiophen-3-yl)methylamine (commercial product).

200 mg of expected product are thus obtained.

MH+=492; Melting point=236-240° C. (Isopropyl ether-dichloromethane)

1H (200 MHz (CD3) 2SO d6, in ppm): 2.06 (m, 2H); 2.69 (s, 3H); 2.78-3.25 (unresolved complex, 4H); 4.78 (m, 1H); 6.30 (d, 1H); 7.19 (t, 2H); 7.60-7.80 (unresolved complex, 4H); 8.10 (d, 1H); 9.51 (broad s, 1H); 9.74 (broad s, 1H).

### Example 7

### N-(1-Benzylpyrrolidin-3-R-yl)-4-[4-(4-fluorophenylamino)pyrimidin-2-ylamino]-N-methylbenzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 200 mg of (1-benzyl-pyrrolidin-3-R-yl)-N-methylamine (commercial product). 318 mg of expected product are thus obtained.

MH+=533; Melting point=154-155° C. (Isopropyl ether-dichloromethane); α_{D}=+24 (C=0.1, MeOH)

1H (200 MHz (CD3) 2SO d6, in ppm): 1.45 (m, 1H); 1.83 (m, 1H); 2.10 (q, 1H); 2.18-2.34 (unresolved complex, 2H); 2.60 (m, 1H); 2.68 (s, 3H); 3.43 (AB, 2H); 4.45 (m, 1H); 6.28 (d, 1H); 7.10-7.33 (unresolved complex, 7H); 7.60 (d, 2H); 7.65-7.78 (unresolved complex, 2H); 7.94 (d, 2H); 8.10 (d, 1H); 9.49 (broad s, 1H); 9.67 (broad s, 1H).

### Example 8

### 4-[4-(4-Fluorophenylamino)pyrimidin-2-ylamino]-N-methyl-N-piperidin-4-yl-benzenesulfonamide

### Stage 1: Preparation of the intermediate 4-({4-[4-(4-Fluorophenylamino)pyrimidin-2-ylamino]benzenesulfonyl}-methylamino)piperidine-1-carboxylic acid tert-butyl ester

The preparation of the intermediate 4-({4-[4-(4-fluoro-phenylamino)pyrimidin-2-ylamino]benzenesulfonyl}methyl-amino)piperidine-1-carboxylic acid tert-butyl ester is first carried out.

The procedure is carried out as in stage 4 of example 1 starting with 800 mg of 4-[4-(4-fluorophenylamino)-pyrimidin-2-ylamino]benzenesulfonyl chloride hydro-chloride which are reacted with 485 mg of methylamino-piperidine-1-carboxylic acid tert-butyl ester. 390 mg of expected product are thus obtained.

MH+=557; Melting point=174-176° C. (Isopropyl ether-dichloromethane)

1H (200 MHz (CD3) 2SO d6, in ppm): 1.14-1.61 (unresolved complex, 13H); 2.61 (s, 3H); 2.70 (m, 2H); 3.70-4.07 (unresolved complex, 3H); 6.27 (d, 1H); 7.16 (t, 2H); 7.57-7.79 (unresolved complex, 4H); 7.94 (d, 2H); 8.06 (d, 1H); 9.46 (bs, 1H); 9.66 (bs, 1H).

### Stage 2: 4-[4-(4-Fluorophenylamino)pyrimidin-2-ylamino]-N-methyl-N-piperidin-4-yl-benzenesulfonamide hydrochloride

300 mg of 4-({4-[4-(4-fluorophenylamino)pyrimidin-2-ylamino]benzenesulfonyl}methylamino)piperidine-1-carboxylic acid tert-butyl ester are kept stirring in 40 ml of 2N hydrochloric ether. After one night, the reaction medium is filtered in order to obtain 220 mg of expected product (finished product example 8).

MH+=457; Melting point=205-210° C. (Isopropyl ether-dichloromethane)

1H (200 MHz (DMSO) d6, in ppm): 1.41 (d, 2H); 1.85 (m, 2H); 2.66 (s, 3H); 2.94 (m, 2H); 3.22 (d, 2H); 4.09 (m, 1H); 6.55 (d, 1H); 7.25 (t, 2H); 7.63 (m, 2H); 7.77 (s, 4H); 8.09 (d, 1H); 8.53-9.08 (unresolved complex, 2H); 11.05 (broad s, 1H); 11.11 (broad s, 1H).

The product of example 8 can serve as intermediate to all the finished products of examples 3, 9, 10, 13, 15, 16, 22, 23, 24, 26, 27, 28 by a reductive amination reaction which would use the same procedure as procedure 2.

### Example 9

### 4-[4-(4-Fluorophenylamino)pyrimidin-2-ylamino]-N-methyl-N-(1-pyridin-2-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 254 mg of methyl(1-pyridin-2-ylmethylpiperidin-4-yl)amine hydrochloride. 205 mg of expected product are thus obtained.

MH+=548; Melting point=202-204° C. (Isopropyl ether-dichloromethane)

1H (200 MHz (CD3) 2SO d6, in ppm): 1.22 (d, 2H); 1.60 (m, 2H); 2.03 (t, 2H); 2.65 (s, 3H); 2.78 (d, 2H); 3.52 (s, 2H); 3.63 (m, 1H); 6.27 (d, 1H); 7.06-7.29 (unresolved complex, 3H); 7.53 (d, 1H); 7.63-7.80 (unresolved complex, 5H); 7.93 (d, 2H); 8.06 (d, 1H); 8.44 (d, 1H); 9.47 (broad s, 1H); 9.66 (broad s, 1H).

### Example 10

### N-(1-Ethylpiperidin-4-yl)-4-[4-(4-fluoro-phenylamino)pyrimidin-2-ylamino]-N-methylbenzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 174 mg of (1-ethylpiperidin-4-yl)methylamine hydrochloride. 205 mg of expected product are thus obtained.

MH+=485; Melting point=162-163° C. (Isopropyl ether-dichloromethane)

1H (200 MHz (CD3) 2SO d6, in ppm): 0.90 (t, 3H); 1.21 (d, 2H); 1.54 (m, 2H); 2.24 (q, 2H); 2.64 (s, 3H); 2.81 (d, 2H); 3.60 (m, 1H); 6.27 (d, 1H); 7.16 (t, 2H); 7.53-7.78 (unresolved complex, 4H); 7.93 (d, 2H); 8.07 (d, 1H); 9.48 (broad s, 1H); 9.66 (broad s, 1H).

### Example 11

### 4-[4-(3,4-Difluorophenylamino)pyrimidin-2-ylamino]-N-methyl-N-(1-methylpiperidin-4-yl)benzene-sulfonamide

### Stage 1: 4-Chloro-N-(3,4-difluorophenyl)pyrimidin-2-amine

The preparation of this compound is carried out according to the same process as for example 1 starting with the reaction of 9.21 g of dichloropyrimidine with 8 g of 3,4-difluoroaniline: 10.3 g of expected product are this obtained.

### Stage 2: N²-(3,4-Difluorophenyl)-N⁴-phenylpyrimidine-2,4-diamine

The preparation of this compound is carried out according to the same process as for example 1 atarting with the reaction of 7 g of (2-chloropyrimidin-4-yl)(3,4-difluorophenyl)amine obtained in stage 1 above with 2.72 g of aniline:

8 g of expected product are thus obtained.

### Stage 3: 4-[4-(3,4-Difluorophenylamino)pyrimidin-2-ylamino]benzenesulfonyl chloride hydrochloride

The preparation of this compound is carried out according to the same process as for example 1 starting with the reaction of 8 g of N*4*-(3,4-difluorophenyl)-N*2*-phenylpyrimidine-2,4-diamine obtained in the stage above with chlorosulfonic acid:

9 g of expected product are thus obtained.

### Stage 4: 4-[4-(3,4-Difluorophenylamino)pyrimidin-2-ylamino]-N-methyl-N-(1-methylpiperidin-4-yl)benzene-sulfonamide

The procedure is carried out as in stage 4 of example 1 starting with 400 mg of 4-[4-(3,4-difluorophenylamino)-pyrimidin-2-ylamino]benzenesulfonyl chloride hydro-chloride which are reacted with 0.17 ml of methyl(1-methylpiperidin-4-yl)amine (commercial product). 110 mg of expected product are thus obtained.

MH+=489; Melting point=181-183° C. (Isopropyl ether-dichloromethane)

1H (200 MHz (CD3) 2SO d6, in ppm): 1.22 (m, 2H); 1.60 (m, 2H); 1.98 (m, 2H); 2.14 (s, 3H); 2.64 (s, 3H); 2.76 (d, 2H); 3.62 (m, 1H); 6.31 (d, 1H); 7.17-7.49 (unresolved complex, 2H); 7.65 (d, 2H); 7.94 (d, 2H); 8.01-8.24 (unresolved complex, 2H); 9.70 (broad s, 1H); 9.77 (broad s, 1H).

### Example 12

### 4-[4-(3-Chloro-4-fluorophenylamino)pyrimidin-2-ylamino]-N-methyl-N-(1-methylpiperidin-4-yl)benzenesulfonamide

### Stage 1: 4-Chloro-N-(3-chloro-4-fluorophenyl)pyrimidin-2-amine

The preparation of this compound is carried out according to the same process as for example 1 starting with the reaction of 10 g of dichloropyrimidine with 9.75 g of 3-chloro-4-fluoroaniline:

11.3 g of expected product are thus obtained.

### Stage 2: N²-(3-Chloro-4-fluorophenyl)-N⁴-phenylpyrimidine-2,4-diamine

The preparation of this compound is carried out according to the same process as for example 1 starting with the reaction of 10 g of (2-chloropyrimidin-4-yl)(3-chloro-4-fluorophenyl)amine obtained in the stage above with 3.61 g of aniline:

13 g of expected product are thus obtained.

### Stage 3: 4-[4-(3-Chloro-4-fluorophenylamino)pyrimidin-2-ylamino]benzenesulfonyl chloride hydrochloride

The preparation of this compound is carried out according to the same process as for example 1 starting with the reaction of 6 g of N*4*-(3-chloro-4-fluorophenyl)-N*2*-phenylpyrimidine-2,4-diamine obtained in the stage above with chlorosulfonic acid:

7 g of expected product are thus obtained.

### Stage 4: 4-[4-(3-Chloro-4-fluorophenylamino)pyrimidin-2-ylamino]-N-methyl-N-(1-methylpiperidin-4-yl)benzene-sulfonamide

The process is carried out as in stage 4 of example 1 starting with 400 mg of 4-[4-(3-chloro-4-fluorophenyl-amino)pyrimidin-2-ylamino]benzenesulfonyl chloride hydrochloride which are reacted with 17 ml of methyl-(1-methylpiperidin-4-yl)amine (commercial product). 250 mg of expected product are thus obtained.

MH+=506; Melting point=183-186° C. (Isopropyl ether-dichloromethane)

1H (200 MHz (CD₃) 2SO d6, in ppm): 1.20 (d, 2H); 1.57 (m, 2H); 1.88 (t, 2H); 2.10 (s, 3H); 2.57-2.82 (unresolved complex, 5H); 3.56 (m, 1H); 6.30 (d, 1H); 7.37 (t, 1H); 7.51 (m, 1H); 7.64 (d, 2H); 7.92 (d, 2H); 8.04 (dd, 1H); 8.13 (d, 1H); 9.66 (broad s, 1H); 9.78 (broad s, 1H).

### Example 13

### 4-[4-(4-Fluorophenylamino)pyrimidin-2-ylamino]-N-methyl-N-(1-thiophen-2-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 261 mg of methyl(1-thiophen-2-ylmethylpiperidin-4-yl)amine hydrochloride. 261 mg of expected product are thus obtained.

MH+=553; Melting point=175-176° C. (Isopropyl ether-dichloromethane)

1H (200 MHz (CD3) 2SO d6, in ppm): 1.26 (d, 2H); 1.56 (m, 2H); 1.96 (t, 2H); 2.65 (s, 3H); 2.81 (d, 2H); 3.48-3.77 (unresolved complex, 3H); 6.23 (d, 1H); 6.84-6.99 (unresolved complex, 2H); 7.16 (t, 2H); 7.38 (dd, 1H); 7.54-7.78 (unresolved complex, 4H); 7.92 (d, 2H); 8.06 (d, 1H); 9.48 (broad s, 1H); 9.66 (broad s, 1H).

### Example 14

### N-Cyclopropyl-4-[4-(4-fluorophenylamino)-pyrimidin-2-ylamino]-N-(1-methylpiperidin-4-yl)benzene-sulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 162 mg of cyclopropyl-(1-methylpiperidin-4-yl)amine (commercial product). 181 mg of expected product are thus obtained.

MH+=497; Melting point=218° C. (Isopropyl ether-dichloromethane)

1H (200 MHz (CD3) 2SO d6, in ppm): 0.62-0.92 (unresolved complex, 4H); 1.33 (m, 2H); 1.62-2.01 (unresolved complex, 5H); 2.09 (s, 3H); 2.72 (d, 2H); 3.62 (m, 1H); 6.28 (d, 1H); 7.16 (t, 2H); 7.57-7.78 (unresolved complex, 4H); 7.95 (d, 2H); 8.07 (d, 2H); 9.48 (broad s, 1H); 9.69 (broad s, 1H).

### Example 15

### N-(1-Benzylpyrrolidin-3-yl)-4-[4-(4-fluoro-phenylamino)pyrimidin-2-ylamino]-N-ethylbenzene-sulfonamide

The procedure is carried out as in stage 4 of example 1 starting with 400 mg of [4-(4-fluorophenylamino)-pyrimidin-2-ylamino]benzenesulfonyl chloride hydro-chloride which are reacted with 205 mg of racemic (1-benzylpyrrolidin-3-yl)ethylamine (commercial product). 152 mg of expected product are thus obtained.

MH+=547; Melting point=125-127° C. (Isopropyl ether-dichloromethane)

1H (200 MHz (CD3) 2SO d6, in ppm): 1.13 (t, 3H); 1.37 (m, 1H); 1.86 (m, 1H); 2.05 (m, 1H); 2.24 (d, 2H); 2.59 (m, 1H); 3.13 (q, 2H); 3.33 (AB, 2H); 4.33 (m, 1H); 6.24 (d, 1H); 7.03-7.30 (unresolved complex, 7H); 7.51-7.74 (unresolved complex, 4H); 7.86 (d, 2H).

### Example 16

### 4-[4-(4-Fluorophenylamino)pyrimidin-2-ylamino]-N-methyl-N-(1-thiophen-3-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 261 mg of methyl(1-thiophen-3-ylmethylpiperidin-4-yl)amine hydrochloride. 225 mg of expected product are thus obtained.

MH+=553; Melting point=173-174° C. (Isopropyl ether-dichloromethane)

1H (200 MHz (CD3) 2SO d6, in ppm): 1.21 (d, 2H); 1.56 (q, 2H); 1.90 (t, 2H); 2.64 (s, 3H); 2.77 (d, 2H); 3.41 (s, 2H); 3.60 (m, 1H); 6.26 (d, 1H); 6.98 (d, 1H); 7.06-7.31 (unresolved complex, 3H); 7.43 (m, 1H); 7.54-7.80 (unresolved complex, 4H); 7.93 (d, 2H); 8.07 (d, 1H); 9.48 (broad s, 1H); 9.66 (broad s, 1H).

### Example 17

### N-(1-Benzylazepan-4-yl)-4-[4-(4-fluoro-phenylamino)-6-methylpyrimidin-2-ylamino]-N-methyl-benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 268 mg of racemic (1-benzylazepan-4-yl)methylamine hydrochloride.

150 mg of expected product are thus obtained.

MH+=561; Melting point=147-148° C. (Isopropyl ether-dichloromethane)

1H (200 MHz (CD3) 2SO d6, in ppm): 1.25-1.75 (unresolved complex, 9H); 2.32-2.59 (unresolved complex, 4H); 2.64 (s, 3H); 3.54 (s, 2H); 3.98 (m, 1H); 6.29 (d, 1H); 7.09-7.38 (unresolved complex, 7H); 7.62 (d, 2H); 7.71 (m, 2H); 7.95 (d, 2H); 8.09 (d, 2H); 9.49 (broad s, 1H); 9.66 (broad s, 1H).

### Example 18

### N-(2-Dimethylaminoethyl)-4-[4-(4-fluoro-phenylamino)pyrimidin-2-ylamino]-N-(1-methylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 255 mg of N,N-dimethyl-N′-(1-methylpiperidin-4-yl)ethane-1,2-diamine hydro-chloride (commercial product).

155 mg of expected product are thus obtained.

MH+=528; Melting point=135-137° C. (Isopropyl ether-dichloromethane)

1H (200 MHz (CD3) 2SO d6, in ppm): 1.31 (d, 2H); 1.57 (m, 2H); 1.83 (t, 2H); 2.08 (s, 3H); 2.15 (s, 6H); 2.39 (t, 2H); 2.71 (d, 2H); 3.13 (t, 2H); 4.48 (m, 1H); 6.28 (d, 1H); 7.16 (t, 2H); 7.55-7.78 (unresolved complex, 4H); 7.92 (d, 2H); 8.07 (d, 1H); 9.48 (broad s, 1H); 9.66 (broad s, 1H).

### Example 19

### 4-[4-(4-Fluorophenylamino)pyrimidin-2-ylamino]-N-(2-hydroxyethyl)-N-(1-methylpiperidin-4-yl)-benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 230 mg of 2-(1-methyl-piperidin-4-ylamino)ethanol hydrochloride (commercial product).

40 mg of expected product are thus obtained.

MH+=501; Melting point=125-135° C. (Isopropyl ether-dichloromethane)

1H (200 MHz (CD3) 2SO d6, in ppm): 1.29 (m, 2H); 1.57 (m, 2H); 1.85 (t, 2H); 2.09 (s, 3H); 2.71 (d, 2H); 3.09 (t, 2H); 3.47 (d, 3H); 4.73 (t, 1H); 6.27 (d, 1H); 7.16 (t, 1H); 7.56-7.78 (unresolved complex, 4H); 7.92 (d, 2H); 8.06 (d, 1H); 9.48 (broad s, 1H); 9.66 (broad s, 1H).

### Example 20

### 4-[4-Chloro-6-(4-fluorophenylamino)pyrimidin-2-ylamino]-N-methyl-N-(1-methylpiperidin-4-yl)benzenesulfonamide

### Stage 1: (2,6-Dichloropyrimidin-4-yl)(4-fluorophenyl)-amine

The preparation of this compound is carried out according to the same process as for example 1 starting with the reaction of 12 g of trichloropyrimidine with 7.38 g of 4-fluoroaniline:

8.7 g of expected product are thus obtained.

### Stage 2: 6-Chloro-N*4*-(4-fluorophenyl)-N*4*-methyl-N*2*-phenylpyrimidine-2,4-diamine

The preparation of this compound is carried out according to the same process as for example 1 starting with the reaction of 4 g of (2,6-dichloropyrimidin-4-yl)(4-fluorophenyl)amine obtained in the stage above with 1.44 g of aniline:

2.5 g of expected product are thus obtained.

### Stage 3: 4-[4-Chloro-6-(4-fluorophenylamino)pyrimidin-2-ylamino]benzenesulfonyl chloride hydrochloride

The preparation of this compound is carried out according to the same process as for example 1 starting with the reaction of 2 g of 6-chloro-N*4*-(4-fluorophenyl)-N*4*-methyl-N*2*-phenylpyrimidine-2,4-diamine obtained at the stage above with chlorosulfonic acid: 2 g of expected product are thus obtained.

### Stage 4: 4-[4-Chloro-6-(4-fluorophenylamino)pyrimidin-2-ylamino]-N-methyl-N-(1-methylpiperidin-4-yl)benzene-sulfonamide

The procedure is carried out as in stage 4 of example 1 starting with 400 mg of 4-[4-chloro-6-(4-fluorophenyl-amino)pyrimidin-2-ylamino]benzenesulfonyl chloride hydrochloride which are reacted with 0.17 ml of methyl-(1-methylpiperidin-4-yl)amine (commercial product). 300 mg of expected product are thus obtained.

MH+=506; Melting point=140-142° C. (Isopropyl ether-dichloromethane)

1H (200 MHz (CD3) 2SO d6, in ppm): 1.19 (m, 2H); 1.57 (m, 2H); 1.86 (t, 2H); 2.08 (s, 3H); 2.57-2.83 (unresolved complex, 5H); 3.60 (m, 1H); 6.26 (s, 1H); 7.20 (t, 2H); 7.48-7.70 (unresolved complex, 4H); 7.84 (d, 2H); 9.71 (broad s, 1H); 10.05 (broad s, 1H).

### Example 21

### 4-[4-(4-Fluorophenylamino)pyrimidin-2-ylamino]-N-methyl-N-(1-methylazepan-4-yl)benzenesulfon-amide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 185 mg of methyl(1-methylazepan-4-yl)amine (commercial product).

1H (200 MHz (CD3) 2SO d6, in ppm): 0.90 (t, 3H); 1.21 (d, 2H); 1.54 (m, 2H); 2.24 (q, 2H); 2.64 (s, 3H); 2.81 (d, 2H); 3.60 (m, 1H); 6.27 (d, 1H); 7.16 (t, 2H); 7.53-7.78 (unresolved complex, 4H); 7.93 (d, 2H); 8.07 (d, 1H); 9.48 (broad s, 1H); 9.66 (broad s, 1H).

214 mg of expected product are thus obtained.

MH+=485; Melting point=122-124° C. (Isopropyl ether-dichloromethane)

### Example 22

### 4-[4-(4-Fluorophenylamino)pyrimidin-2-ylamino]-N-methyl-N-(1-pyridyl-3-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 254 mg of methyl(1-pyridyl-3-ylmethylpiperidin-4-yl)amine hydrochloride. 155 mg of expected product are thus obtained.

MH+=548; Melting point=215.8° C. (Isopropyl ether-dichloromethane)

1H (200 MHz (CD3) 2SO d6, in ppm): 1.11-2.36 (unresolved complex, 4H); 2.63 (s, 3H); 2.82-4.60 (unresolved complex, 7H); 6.30 (d, 1H); 7.17 (t, 2H); 7.28-7.83 (unresolved complex, 5H); 7.96 (d, 2H); 8.07 (d, 1H); 8.36-9.24 (unresolved complex, 2H); 9.59 (s, 1H); 9.71 (s, 1H).

### Example 23

### 4-[4-(4-Fluorophenylamino)-6-methylpyrimidin-2-ylamino]-N-methyl-N-(1-thiazol-2-ylmethyl-piperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 260 mg of methyl(1-thiazol-2-ylmethylpiperidin-4-yl)amine hydrochloride. 165 mg of expected product are thus obtained.

MH+=554; Melting point=220° C. (Isopropyl ether-dichloromethane)

1H (200 MHz (CD3) 2SO d6, in ppm): 1.2 (d, 2H); 1.60 (q, 2H); 2.14 (t, 2H); 2.67 (s, 3H); 2.86 (d, 2H); 3.66 (m, 1H); 3.78 (s, 2H); 6.28 (d, 1H); 7.18 (t, 2H); 7.75-7.77 (unresolved complex, 6H); 7.95 (d, 2H); 8.08 (d, 1H); 9.48 (broad s, 1H); 9.67 (broad s, 1H).

### Example 24

### 4-[4-(4-Fluorophenylamino)pyrimidin-2-ylamino]-N-methyl-N-(1-pyridyl-4-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 254 mg of methyl(1-pyridyl-4-ylmethylpiperidin-4-yl)amine hydrochloride. 205 mg of expected product are thus obtained.

MH+=548; Melting point=205.3° C. (Isopropyl ether-dichloromethane)

1H (200 MHz (CD3) 2SO d6, in ppm): 1.52 (d, 2H); 1.99 (m, 2H); 2.68 (s, 3H); 3.19 (m, 2H); 3.41 (d, 2H); 4.12 (m, 1H); 4.60 (s, 2H); 6.51 (d, 1H); 7.21 (t, 2H); 7.75-7.87 (unresolved complex, 6H); 8.05 (d, 1H); 8.22 (d, 2H); 9.06 (d, 2H); 11.00 (s, 1H); 11.26 (s, 1H).

### Example 25

### 2-{4-[4-(4-Fluorophenylamino)pyrimidin-2-ylamino]benzenesulfonylamino}-N-methyl-N-(1-methyl-piperidin-4-yl)acetamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 273 mg of 2-amino-N-methyl-N-(1-methylpiperidin-4-yl)acetamide hydrochloride. 260 mg of expected product are thus obtained.

MH+=528; Melting point=233-234.4° C. (Isopropyl ether-dichloromethane)

1H (200 MHz (CD3) 2SO d6, in ppm): 1.44-2.08 (unresolved complex, 4H); 2.54-3.53 (unresolved complex, 10H); 3.60-4.90 (unresolved complex, 3H); 6.48 (d, 1H); 7.23 (t, 2H); 7.45-7.73 (unresolved complex, 4H); 7.80 (d, 2H); 8.03 (d, 1H); 10.97 (s, 1H); 11.16 (s, 1H).

### Example 26

### 4-[4-(4-Fluorophenylamino)pyrimidin-2-ylamino]-N-methyl-N-(1-pirazin-2-ylmethylpiperidin-4-yl)benzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 294 mg of methyl(1-pirazin-2-ylmethylpiperidin-4-yl)amine hydrochloride. 80 mg of expected product are thus obtained.

MH+=548; Melting point=180° C. (Isopropyl ether-dichloromethane)

1H (200 MHz (CD3) 2SO d6, in ppm): 1.21 (d, 2H); 1.58 (m, 2H); 2.06 (t, 2H); 2.63 (s, 3H); 2.78 (d, 2H); 3.48-3.74 (unresolved complex, 3H); 6.26 (d, 1H); 7.15 (t, 2H); 7.50-7.77 (unresolved complex, 4H); 7.92 (d, 2H); 8.06 (d, 1H); 8.42-8.66 (unresolved complex, 3H); 9.46 (s, 1H); 9.97-10.81 (bs, 1H).

### Example 27

### 4-[4-(4-Fluorophenylamino)-6-methyl-pyrimidin-2-ylamino]-N-(1-furan-3-ylmethylpiperidin-4-yl)-N-methylbenzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 277 mg of (1-furan-3-ylmethylpiperidin-4-yl)methylamine hydrochloride. 220 mg of expected product are thus obtained.

MH+=537; Melting point=156-156° C. (Isopropyl ether-dichloromethane)

1H (200 MHz (CD3) 2SO d6, in ppm): 1.19 (d, 2H); 1.53 (q, 3.56 (m, 1H); 6.21 (d, 1H); 6.26 (d, 1H); 6.35 (t, 1H); 7.15 (t, 2H); 7.53 (s, 1H); 7.61 (d, 2H); 7.68 (m, 2H); 7.92 (d, 2H); 8.06 (d, 1H).

### Example 28

### 4-[4-(4-Fluorophenylamino)-6-methylpyrimidin-2-ylamino]-N-(1H-imidazol-2-ylmethylpiperidin-4-yl)-N-methylbenzenesulfonamide

The procedure is carried out as in stage 4 of example 1 starting with 400 mg of 4-[4-(4-fluorophenylamino)-pyrimidin-2-ylamino]benzenesulfonyl chloride hydro-chloride which are reacted with 277 mg of [1-(1H-imidazol-2-ylmethyl)piperidin-4-yl]methylamine hydro-chloride.

246 mg of expected product are thus obtained.

### Reference Example A

### 4-(2-tert-Butoxycarbonylaminoethylamino)piperidine-1-carboxylic acid tert-butyl ester

2 g of 4-oxopiperidine-1-carboxylic acid tert-butyl ester and 1.6 g of (2-aminoethyl)carbamic acid tert-butyl ester are dissolved in 20 ml of THF. 1.5 g of sodium triacetoxyborohydride are added. The reaction medium is kept stirring at room temperature overnight. 20 ml of methanol are added to the reaction medium and then the medium is heated at 70° C. for 1 h 30 min. After concentrating to dryness and taking up in a sodium hydroxide solution, the medium is extracted with dichloromethane and the chlorinated phase is washed with saturated NaCl and dried over Na2SO4. After concentrating to dryness, 1.7 g of expected product are obtained.

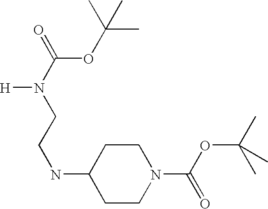

### 4-(2-Dimethylaminoethylamino)piperidine-1-carboxylic acid tert-butyl ester

The procedure is carried out as in example A starting with 2 g of 4-oxopiperidine-1-carboxylic acid tert-butyl ester and 920 mg of N*1*,N*1*-dimethylethane-1,2-diamine. 1.3 g of expected product are obtained.

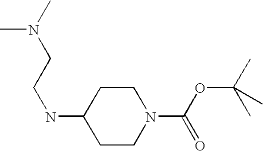

### 4-(2-Diethylaminoethylamino)piperidine-1-carboxylic acid tert-butyl ester

The procedure is carried out as in example A starting with 2 g of 4-oxopiperidine-1-carboxylic acid tert-butyl ester and 1.22 g of N*1*,N*1*-diethylethane-1,2-diamine. 1.35 g of expected product are obtained.

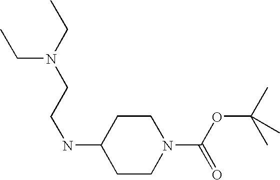

### 4-(2-Pyrrolidin-1-ylethylamino)piperidine-1-carboxylic acid tert-butyl ester

The procedure is carried out as in example A starting with 2 g of 4-oxopiperidine-1-carboxylic acid tert-butyl ester and 1.2 g of 2-pyrrolidin-1-ylethylamine. 1.17 g of expected product are obtained.

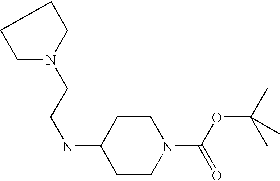

### Methyl[2-(1-methylpiperidin-4-ylamino)ethyl]carbamic acid tert-butyl ester

The procedure is carried out as in example A starting with 2 g of 1-methylpiperidin-4-one 2.05 g of (2-amino-ethyl)methylcarbamic acid tert-butyl ester.

550 mg of expected product are obtained.

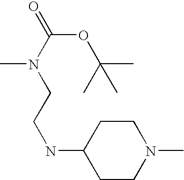

### [2-(1-Methylpiperidin-4-ylamino)ethyl]carbamic acid tert-butyl ester

The procedure is carried out as in example A starting with 2 g of 1-methylpiperidin-4-one and 2.88 g of (2-aminoethyl)carbamic acid tert-butyl ester.

950 mg of expected product are thus obtained.

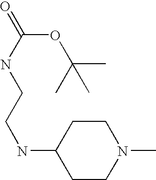

### Example 29

### 4-[4-(4-Fluorophenylamino)pyrimidin-2-ylamino]-N-[1-(1-N-oxidepyridin-4-ylmethyl)piperidin-4-yl]-N-methylbenzenesulfonamide

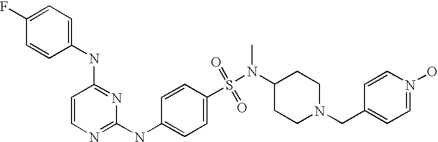

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 67 mg of 1-N-oxidepyridine-4-carbaldehyde.

225 mg of expected product are thus obtained.

Alternatively, 260 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (135 mg) and 1-N-oxidepyridine-4-carbaldehyde (80 mg). After treatment, 135 mg of expected product are isolated.

MH+=564; Melting point=155-156° C. (Trituration in isopropyl ether)

1H NMR (DMSO): 1.22 (d, 2); 1.56 (q, 2); 1.97 (t, 2); 2.64 (s, 3); 2.72 (d, 2); 3.37 (s, 2); 3.62 (t, 1); 6.26 (d, 1); 7.16 (t, 2); 7.25 (d, 2); 7.61 (d, 2); 7.68 (m, 2); 7.92 (d, 2); 8.03-8.15 (ml, 3); 9.45 (s, 1); 9.65 (s, 1).

### Example 30

### 4-[4-(4-Fluorophenylamino)pyrimidin-2-ylamino]-N-methyl-N-[1-(2-methyl-3H-imidazol-4-ylmethyl)piperidin-4-yl]benzenesulfonamide

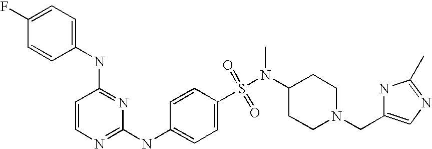

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 60 mg of 2-methyl-3H-imidazole-4-carbal-dehyde.

190 mg of expected product are thus obtained.

Alternatively, 410 mg of 4-[4-(4-fluorophenylamino)-pyrimidin-2-ylamino]-N-methyl-N-piperidin-4-yl-benzene-sulfonamide (base) are placed in THF (25 ml) and the medium is stirred at RT overnight in the presence of NaHB(OAc)3 (300 mg) and 2-methyl-3H-imidazole-4-carbaldehyde (120 mg). 200 mg of additional NaHB(OAc)3 are then added and the medium is heated at 70° C. for 2 hours. After treatment, 324 mg of expected product are isolated.

MH+=551; Melting point=155° C. (Trituration in isopropyl ether)

1H NMR (DMSO): 1.22 (m, 2); 1.53 (m, 2); 1.89 (t, 2); 2.18 (s, 3); 2.64 (s, 3); 2.79 (m, 2); 3.22 (s, 2); 3.60 (m, 1); 6.29 (d, 1); 6.43-6.80 (bs, 1); 7.18 (t, 2); 7.55-7.78 (unresolved complex, 4); 7.95 (d, 2); 8.09 (d, 1); 9.49 (s, 1); 9.67 (s, 1); 11.34-11.61 (s, 1).

### Example 31

### N-[1-(2-Fluorobenzyl)piperidin-4-yl]-4-[4-(4-fluorophenylamino)pyrimidin-2-ylamino]-N-methyl-benzenesulfonamide

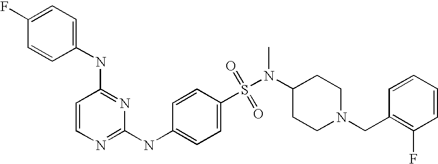

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 66 mg of 2-fluorobenzaldehyde. 210 mg of expected product are thus obtained.

Alternatively, 410 mg of 4-[4-(4-fluorophenylamino)-pyrimidin-2-ylamino]-N-methyl-N-piperidin-4-ylbenzene-sulfonamide (base) are placed in THF (15 ml) and the medium is stirred at RT overnight in the presence of NaHB(OAc)3 (300 mg) and 2-fluorobenzaldehyde (0.1 ml). After treatment, 386 mg of expected product are isolated.

MH+=565; Melting point=182-183° C. (Trituration in isopropyl ether)

1H NMR (DMSO): 1.22 (d, 2); 1.57 (m, 2); 1.99 (t, 2); 2.65 (s, 3); 2.78 (d, 2); 3.47 (s, 2); 3.62 (m, 1); 6.29 (d, 1); 7.06-7.42 (unresolved complex, 6); 7.55-7.78 (unresolved complex, 4); 7.95 (d, 2); 8.09 (d, 1); 9.49 (s, 1); 9.67 (s, 1).

### Example 32

### N-[1-(3-Fluorobenzyl)piperidin-4-yl]-4-[4-(4-fluorophenylamino)pyrimidin-2-ylamino]-N-methyl-benzenesulfonamide

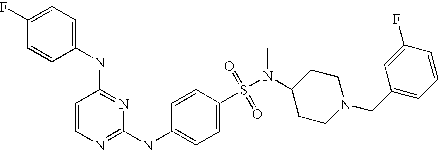

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 66 mg of 3-fluorobenzaldehyde.

195 mg of expected product are thus obtained.

Alternatively, 410 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (300 mg) and 3-fluorobenzaldehyde (0.1 ml). After treatment, 351 mg of expected product are isolated.

MH+=565; Melting point=207° C. (Trituration in isopropyl ether)

1H NMR (DMSO): 1.23 (d, 2); 1.59 (m, 2); 1.96 (t, 2); 2.66 (s, 3); 2.76 (d, 2); 3.43 (s, 2); 3.64 (m, 1); 6.29 (d, 1); 6.95-7.43 (unresolved complex, 6); 7.55-7.78 (unresolved complex, 4); 7.94 (d, 2); 8.08 (d, 1); 9.49 (s, 1); 9.67 (s, 1).

### Example 33

### N-[1-(4-Fluorobenzyl)piperidin-4-yl]-4-[4-(4-fluorophenylamino)pyrimidin-2-ylamino]-N-methyl-benzenesulfonamide

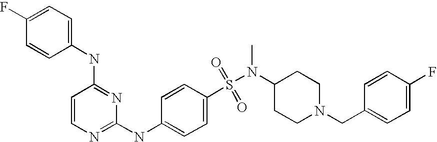

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 66 mg of 4-fluorobenzaldehyde.

200 mg of expected product are thus obtained.

MH+=565; Melting point=129-131° C. (Trituration in isopropyl ether)

1H NMR (DMSO): 1.21 (d, 2); 1.55 (m, 2); 1.92 (t, 2); 2.63 (s, 3); 2.72 (d, 2); 3.37 (s, 2); 3.60 (m, 1); 6.26 (d, 1); 7.00-7.20 (m, 4); 7.26 (dd, 2); 7.61 (d, 2); 7.67 (dd, 2); 7.92 (d, 2); 8.06 (d, 1); 9.46 (s, 1); 9.62 (s, 1).

### Example 34

### 4-[4-(4-Fluorophenylamino)pyrimidin-2-yl-amino]-N-methyl-N-[1-(1-methyl-1H-imidazol-2-ylmethyl)-piperidin-4-yl]benzenesulfonamide

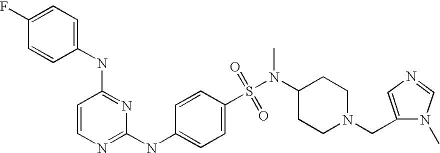

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 60 mg of 1-methyl-1H-imidazole-5-carbal-dehyde.

187 mg of expected product are thus obtained.

Alternatively, 300 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (250 mg) and 1-methyl-1H-imidazole-5-car-baldehyde (90 mg). After treatment, 130 mg of expected product are isolated.

MH+=551; Melting point=274-275° C. (Trituration in isopropyl ether)

1H NMR (DMSO): 1.20 (m, 2); 1.50 (q, 2); 1.89 (t, 2); 2.62 (s, 3); 2.74 (d, 2); 3.33 (s, 2); 3.53 (s, 3); 3.58 (m, 1); 6.26 (d, 1); 6.79 (s, 1); 7.16 (t, 2); 7.48 (s, 1); 7.61 (d, 2); 7.68 (dd, 2); 7.92 (d, 2); 8.06 (d, 1); 9.47 (s, 1); 9.65 (s, 1).

### Example 35

### 4-[4-(4-Fluorophenylamino)pyrimidin-2-ylamino]-N-methyl-N-(1-quinolin-3-ylmethylpiperidin-4-yl)benzenesulfonamide

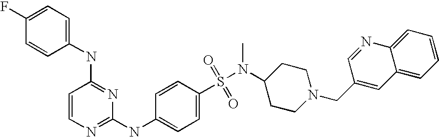

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 84 mg of quinoline-3-carbaldehyde.

254 mg of expected product are thus obtained.

MH+=547; Melting point=125-127° C. (Trituration in isopropyl ether)

1H NMR (DMSO): 1.27 (d, 2); 1.62 (q, 2); 2.05 (t, 2); 2.67 (s, 3); 2.84 (d, 2); 3.65 (bs, 3); 6.28 (d, 1); 7.18 (t, 2); 7.54-7.79 (m, 6); 7.88-7.96 (m, 4); 8.07 (d, 1); 8.1 (s, 1); 8.80 (s, 1); 9.4 (s, 1); 9.6 (s, 1).

### Example 36

### 4-[4-(3-Chloro-4-fluorophenylamino)pyrimidin-2-ylamino]-N-[1-(4-fluorobenzyl)piperidin-4-yl]-N-methylbenzenesulfonamide

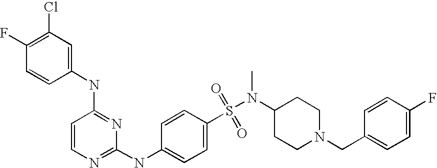

The procedure is carried out by a reductive amination reaction starting with 600 mg of 4-[4-(3-chloro-4-fluorophenylamino)pyrimidin-2-ylamino]benzenesulfonyl hydrochloride (product obtained in stage 3 of example 12) which are reacted with 66 mg of 4-fluorobenzaldehyde.

350 mg of expected product are thus obtained.

MH+=553; Melting point=175-176° C. (Trituration in isopropyl ether)

1H NMR (DMSO): 1.20 (d, 2); 1.54 (q, 2); 1.90 (t, 2); 2.63 (s, 3); 2.72 (d, 2); 3.36 (s, 2); 3.56 (t, 1); 6.28 (d, 1); 7.09 (t, 2); 7.26 (m, 2); 7.36 (t, 1); 7.50 (m, 1); 7.63 (d, 2); 7.90 (d, 2); 8.03 (d, 1); 8.10 (d, 1); 9.64 (s, 1); 9.75 (s, 1).

### Example 37

### 4-[4-(4-Fluorophenylamino)pyrimidin-2-yl-amino]-N-(1-isopropylpiperidin-4-yl)-N-methylbenzene-sulfonamide

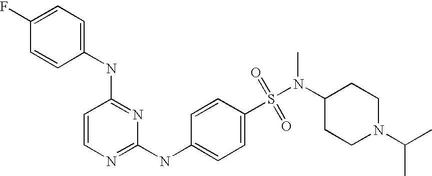

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 38 mg of propan-2-one.

180 mg of expected product are thus obtained.

Alternatively, 300 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (200 mg) and propan-2-one (0.15 ml). After treatment, 116 mg of expected product are isolated.

MH+=499; Melting point=202-203° C. (Trituration in isopropyl ether)

1H NMR (DMSO): 0.90 (d, 6); 1.24 (d, 2); 1.51 (m, 2); 2.08 (t, 2); 2.56-2.68 (unresolved complex, 4); 2.73 (d, 2); 3.58 (m, 1); 6.28 (d, 1); 7.17 (t, 2); 7.63 (d, 2); 7.69 (m, 2); 7.93 (d, 2); 8.07 (d, 1); 9.46 (s, 2); 9.64 (s, 2).

### Example 38

### 4-[4-(4-Fluorophenylamino)pyrimidin-2-yl-amino]-N-(1-isobutylpiperidin-4-yl)-N-methylbenzene-sulfonamide

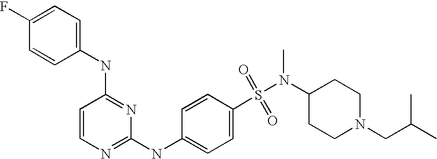

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 48 mg of 2-methylpropionaldehyde. 210 mg of expected product are thus obtained.

Alternatively, 230 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (200 mg) and 2-methylpropionaldehyde (50 mg). After treatment, 200 mg of expected product are isolated.

MH+=513; Melting point=194-195° C. (Trituration in isopropyl ether)

1H NMR (DMSO): 0.79 (d, 6); 1.22 (d, 2); 1.55 (m, 2); 1.66 (bs, 1); 1.83 (t, 2); 1.95 (d, 2); 2.65 (s, 3); 2.76 (d, 2); 3.60 (m, 1); 6.28 (d, 1); 7.17 (t, 2); 7.63 (d, 2); 7.69 (m, 2); 7.93 (d, 2); 8.07 (d, 1); 9.48 (s, 1); 9.67 (s, 1).

### Example 39

### 4-[4-(4-Fluorophenylamino)pyrimidin-2-yl-amino]-N-methyl-N-[1-(3-methylbutyl)piperidin-4-yl]-benzenesulfonamide

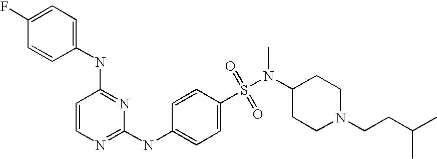

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 56 mg of 3-methylbutyraldehyde. 218 mg of expected product are thus obtained.

Alternatively, 320 mg of 4-[4-(4-fluorophenylamino)-pyrimidin-2-ylamino]-N-methyl-N-piperidin-4-ylbenzene-sulfonamide (base) are placed in THF (10 ml) and the medium is stirred at RT overnight in the presence of NaHB(OAc)3 (250 mg) and 3-methylbutyraldehyde (0.1 ml). After treatment, 258 mg of expected product are isolated.

MH+=527; Melting point=184-185° C. (Trituration in isopropyl ether)

1H NMR (DMSO): 0.77 (d, 6); 1.08-1.27 (unresolved complex, 4); 1.36-1.61 (unresolved complex, 3); 1.78 (t, 2); 2.15 (t, 2); 2.60 (s, 3); 2.75 (d, 2); 3.55 (multiplet, 1); 6.04 (d, 1); 7.52-7.74 (unresolved complex, 4); 7.90 (d, 2); 8.04 (d, 1); 9.44 (s, 2); 9.62 (s, 2).

### Example 40

### 4-[4-(4-Fluorophenylamino)pyrimidin-2-yl-amino]-N-methyl-N-[1-(4,4,4-trifluorobutyl)piperidin-4-yl]benzenesulfonamide

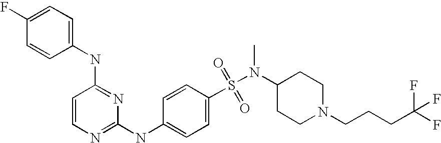

The procedure is carried out by a reductive amination reaction starting with 300 mg of 4-[4-(4-fluorophenyl-amino)pyrimidin-2-ylamino]-N-methyl-N-piperidin-4-yl-benzenesulfonamide hydrochloride (example 8) which are reacted with 82 mg of 4,4,4-trifluorobutyraldehyde. 195 mg of expected product are thus obtained.

Alternatively, 380 mg of 4-[4-(4-fluorophenylamino)pyr-imidin-2-ylamino]-N-methyl-N-piperidin-4-ylbenzene-sulfonamide (base) are placed in THF (10 ml) and the medium is stirred at RT overnight in the presence of NaHB(OAc)3 (300 mg) and 4,4,4-trifluorobutyraldehyde (140 mg). After treatment, 330 mg of expected product are isolated.

MH+=567; Melting point=166-167° C. (Trituration in isopropyl ether)

1H NMR (DMSO): 1.23 (d, 2); 1.42-1.69 (unresolved complex, 4); 1.89 (t, 2); 2.04-2.35 (unresolved complex, 4); 2.65 (s, 3); 2.79 (d, 2); 3.62 (multiplet, 1); 6.29 (d, 1); 7.18 (t, 2); 7.56-7.82 (unresolved complex, 4); 7.95 (d, 2); 8.09 (d, 1); 9.49 (s, 2); 9.67 (s, 2).

### Example 41

### N-(2-Aminoethyl)-4-[4-(4-fluorophenyl-amino)pyrimidin-2-ylamino]-N-(1-methylpiperidin-4-yl)-benzenesulfonamide hydrochloride

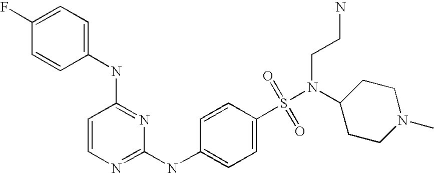

The procedure is carried out as in stage 4 of example 1 starting with 400 mg of 4-[4-(4-fluorophenylamino)-pyrimidin-2-ylamino]benzenesulfonyl chloride hydro-chloride (product obtained in stage 3 of example 1) which are reacted with 230 mg of [2-(1-methylpiperidin-4-ylamino)ethyl]carbamic acid tert-butyl ester.

168 mg of expected product are thus obtained after a decarboxylation reaction according to procedure 2 of example 8.

Likewise, by treating the preceding carbamate (410 mg) dissolved in MeOH (10 ml) with 2M hydrochloric ether (30 ml) overnight at RT, after evaporation of the solvents at RT in a Büchi rotary evaporator, a solid is obtained after trituration in ether (367 mg).

MH+=500; Melting point=225° C. (Trituration in isopropyl ether)

1H NMR (DMSO): 1.63 (d, 2); 2.00 (m, 2); 2.65 (d, 3); 2.80-3.90 (unresolved complex, 8); 4.02 (t, 1); 6.56 (d, 1); 7.28 (t, 2); 7.58-7.75 (unresolved complex, 2); 7.85 (Systeme AA′BB′, 4); 8.05-8.40 (unresolved complex, 4); 10.63-11.33 (m, 3).

### Example 42

### N-(2-Dimethylaminoethyl)-4-[4-(4-fluoro-phenylamino)pyrimidin-2-ylamino]-N-piperidin-4-yl-benzenesulfonamide hydrochloride

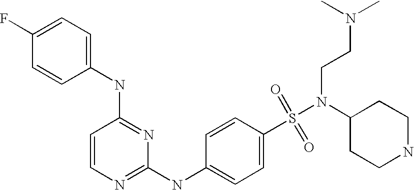

The procedure is carried out as in stage 4 of example 1 starting with 400 mg of 4-[4-(4-fluorophenylamino)-pyrimidin-2-ylamino]benzenesulfonyl chloride hydro-chloride (product obtained in stage 3 of example 1) which are reacted with 241 mg of 4-(2-dimethylamino-ethylamino)piperidine-1-carboxylic acid tert-butyl ester.

132 mg of expected product are thus obtained after a decarboxylation reaction according to procedure 2 of example 8.

Likewise, by treating the preceding carbamate (450 mg) dissolved in MeOH (5 ml) with 2M hydrochloric ether (25 ml) overnight at RT, after evaporation of the solvents at RT in a Büchi rotary evaporator, a solid is obtained after trituration in ether (377 mg).

MH+=514; Melting point=220° C.

1H NMR (DMSO): 1.50 (m, 2); 2.00 (m, 2); 2.82 (s, 6); 2.85-3.90 (unresolved complex, 8); 4.03 (t, 1); 6.51 (d, 1); 7.26 (t, 2); 7.64 (dd, 2); 7.84 (dd, 4); 8.09 (d, 1); 8.71-9.19 (ml, 2); 10.61-11.19 (bs, 3).

### Example 43

### N-(2-Diethylaminoethyl)-4-[4-(4-fluoro-phenylamino)pyrimidin-2-ylamino]-N-piperidin-4-yl-benzenesulfonamide

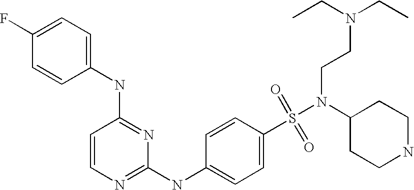

The procedure is carried out as in stage 4 of example 1 starting with 400 mg of 4-[4-(4-fluorophenylamino)-pyrimidin-2-ylamino]benzenesulfonyl chloride hydro-chloride (product obtained in stage 3 of example 1) which are reacted with 265 mg of 4-(2-diethylamino-ethylamino)piperidine-1-carboxylic acid tert-butyl ester.

120 mg of expected product are thus obtained after a decarboxylation reaction according to procedure 2 of example 8.

Likewise, by treating the preceding carbamate (210 mg) dissolved in MeOH (3 ml) with 2M hydrochloric ether (20 ml) overnight at RT, after evaporation of the solvents at RT in a Büchi rotary evaporator, a solid is obtained after trituration in ether (150 mg).

MH+=514; Melting point=210° C. (Trituration in isopropyl ether)

1H NMR (DMSO): 1.24 (t, 6); 1.51 (d, 2); 1.98 (q, 2); 2.92 (q, 2); 3.05-3.93 (unresolved complex, 10); 4.02 (t, 1); 6.51 (d, 1); 7.26 (t, 2); 7.64 (dd, 2); 7.84 (dd, 4); 8.09 (d, 1); 8.71-9.19 (ml, 2); 10.61-11.19 (bs, 3).

### Example 44

### N-(2-Aminoethyl)-4-[4-(4-fluorophenyl-amino)pyrimidin-2-ylamino]-N-piperidin-4-ylbenzene-sulfonamide hydrochloride

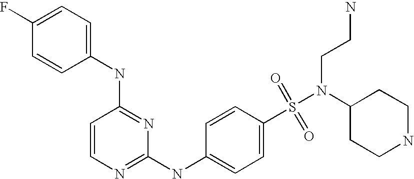

The procedure is carried out as in stage 4 of example 1 starting with 400 mg of 4-[4-(4-fluorophenylamino)-pyrimidin-2-ylamino]benzenesulfonyl chloride hydrochloride (product obtained in stage 3 of example 1) which are reacted with 304 mg of 4-(2-tert-butoxycarbonylaminoethylamino)piperidine-1-carboxylic acid tert-butyl ester.

200 mg of expected product are thus obtained after a decarboxylation reaction according to procedure 2 of example 8.

MH+=486; Melting point=270° C. (Trituration in isopropyl ether)

1H NMR (DMSO): 1.57 (m, 2); 1.85 (m, 2); 2.95 (m, 4); 3.01-3.93 (unresolved complex, 4); 4.03 (t, 1); 6.55 (d, 1); 7.26 (t, 2); 7.64 (dd, 2); 7.80 (s, 4); 8.10 (d, 1); 8.18 (bs, 3); 8.97 (s, 2); 11.03 (bs, 2).

### Example 45

### 4-[4-(4-Fluorophenylamino)pyrimidin-2-ylamino]-N-piperidin-4-yl-N-(2-pyrrolidin-1-ylethyl)-benzenesulfonamide hydrochloride

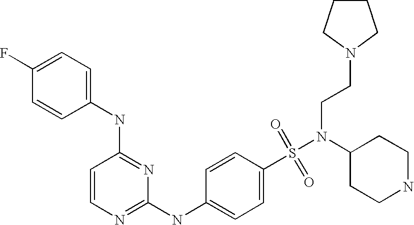

The procedure is carried out as in stage 4 of example 1 starting with 400 mg of 4-[4-(4-fluorophenylamino)-pyrimidin-2-ylamino]benzenesulfonyl chloride hydrochloride (product obtained in stage 3 of example 1) which are reacted with 264 mg of 4-(2-pyrrolidin-1-ylethylamino)piperidine-1-carboxylic acid tert-butyl ester.

115 mg of expected product are thus obtained after a decarboxylation reaction according to procedure 2 of example 8 in the presence of a few drops of MeOH.

MH+=540; Melting point=200° C. (Trituration in isopropyl ether)

1H NMR (DMSO): 1.54 (m, 2); 1.95 (m, 6); 2.66-3.9 (unresolved complex, 12); 4.04 (t, 1); 6.53 (d, 1); 7.28 (t, 2); 7.63 (m, 2); 7.84 (dd, 4); 8.10 (d, 1); 8.73-9.20 (ml, 2); 10.75-11.29 (bs, 3).

### Example 46

### 4-[4-(4-Fluorophenylamino)pyrimidin-2-yl-amino]-N-(2-methylaminoethyl)-N-(1-methylpiperidin-4-yl)benzenesulfonamide hydrochloride

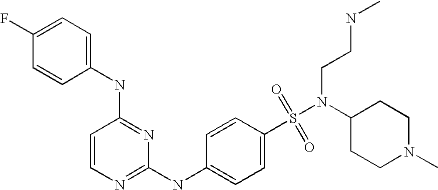

The procedure is carried out as in stage 4 of example 1 starting with 420 mg of 4-[4-(4-fluorophenylamino)-pyrimidin-2-ylamino]benzenesulfonyl chloride hydro-chloride (product obtained in stage 3 of example 1) which are reacted with 300 mg of methyl[2-(1-methylpiperidin-4-ylamino)ethyl]carbamic acid tert-butyl ester.

219 mg of expected product are thus obtained after a decarboxylation reaction according to procedure 2 of example 8.

MH+=514; Melting point=220° C. (Trituration in isopropyl ether)

1H NMR (DMSO): 1.54 (m, 2); 1.95 (m, 6); 2.66-3.9 (unresolved complex, 12); 4.04 (t, 1); 6.53 (d, 1); 7.28 (t, 2); 7.63 (m, 2); 7.84 (dd, 4); 8.10 (d, 1); 8.73-9.20 (ml, 2); 10.75-11.29 (bs, 3).

### Example 47

### N-(4-Aminocyclohexyl)-4-[4-(4-fluorophenyl-amino)pyrimidin-2-ylamino]-N-methylbenzenesulfonamide hydrochloride

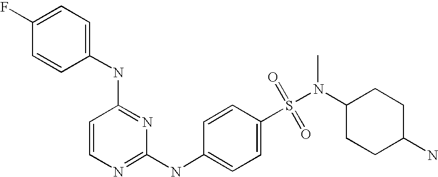

The procedure is carried out as in stage 4 of example 1 starting with 400 mg of 4-[4-(4-fluorophenylamino)-pyrimidin-2-ylamino]benzenesulfonyl chloride hydro-chloride (product obtained in stage 3 of example 1) which are reacted with 202 mg of (4-methylamino-cyclohexyl)carbamic acid tert-butyl ester.

224 mg of expected product are thus obtained, in the form of 2 enantiomers, after a decarboxylation reaction according to procedure 2 of example 8.

This compound 47 is in the form of a 60/40 mixture of two cis and trans isomers and is used as starting material in the reductive amination reaction for the synthesis of the compounds of examples 65 to 85 and of examples 157 and 158.

MH+=571; Melting point=232-233° C. (Trituration in isopropyl ether)

1H NMR (DMSO): 1.0 to 2.05 (unresolved complex, 8); 2.57 to 2.77 (bs, 3); 2.79-3.3 (ml, 1); 3.66 (m, 1); 6.54 (d, 1); 7.24 (t, 2); 7.62 (m, 2); 7.74 (s, 4); 8.08 (bd, 4); 11.11 (s, 2).

### Example 48

### N-(2-Aminoethyl)-4-[4-(4-fluoro-3-methyl-phenylamino)pyrimidin-2-ylamino]-N-piperidin-4-yl-benzenesulfonamide

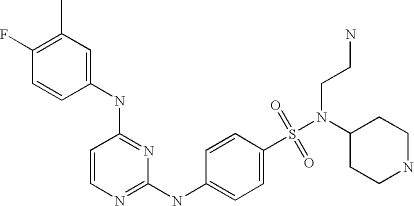

The procedure is carried out as in stage 4 of example 1 starting with 400 mg of 4-[4-(4-fluorophenylamino)-pyrimidin-2-ylamino]benzenesulfonyl chloride hydro-chloride.

### Stage 1: (2-Chloropyrimidin-4-yl)(4-fluoro-3-methyl-phenyl)amine

The preparation of this compound is carried out according to the same process as for example 1 starting with the reaction of 5.3 g of 4-fluoro-3-methylphenylamine with 6.3 g of 2,4-dichloropyrimidine: 3.8 g of expected product are obtained (Melting point=130-131° C.) (Trituration in isopropyl ether).

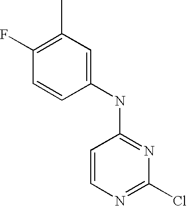

### Stage 2: N*4*-(4-Fluoro-3-methylphenyl)-N*2*-phenyl-pyrimidine-2,4-diamine

The preparation of this compound is carried out according to the same process as for example 1 starting with the reaction of 2.8 g of (2-chloropyrimidin-4-yl)(4-fluoro-3-methylphenyl)amine obtained above and 1.2 ml of aniline: 2.2 g of expected product are obtained (Melting point=134-135° C.) (Trituration in isopropyl ether).

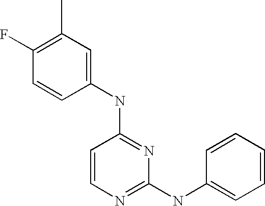

### Stage 3: 4-[4-(4-Fluoro-3-methylphenylamino)pyrimidin-2-ylamino]benzenesulfonyl chloride hydrochloride

The preparation of this compound is carried out according to the same process as for example 1 starting with the reaction of 2 g of N*4*-(4-fluoro-3-methylphenyl)-N*2*-phenylpyrimidine-2,4-diamine obtained above with chlorosulfonic acid: 1.5 g of expected product are obtained.

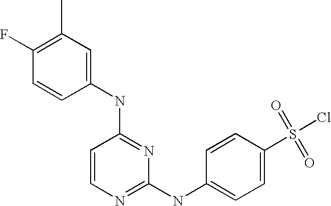

### Stage 4: 4-((2-tert-Butoxycarbonylaminoethyl){4-[4-(4-fluoro-3-methylphenylamino)pyrimidin-2-ylamino]benzene-sulfonyl}amino)piperidine-1-carboxylic acid tert-butyl ester

The procedure is carried out as in stage 4 of example 1 starting with 400 mg of 4-[4-(4-fluoro-3-methylphenyl-amino)pyrimidin-2-ylamino]benzenesulfonyl chloride hydrochloride obtained above which are reacted with 304 mg of 4-(2-tert-butoxycarbonylaminoethylamino)-piperidine-1-carboxylic acid tert-butyl ester: 120 mg of an intermediate are thus obtained, which intermediate gives the expected product after a decarboxylation reaction according to procedure 2 of example 8.

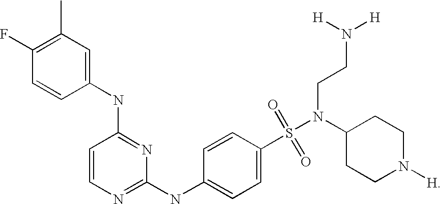

MH+=500; Melting point=242-244° C. (Trituration in isopropyl ether)

1H NMR (DMSO): 1.56 (m, 2); 1.82 (m, 2); 2.14 (s, 3); 2.68-4.21 (unresolved complex, 9); 6.50 (d, 1); 7.16 (t, 1); 7.40 (m, 1); 7.55 (m, 1); 7.90 (s, 4); 8.03-8.2 (bd, 4); 8.9 (bs, 2); 10.60-11.25 (bs, 2).

### Example 49

### 4-[4-(4-Fluorophenylamino)pyrimidin-2-yl-amino]-N-[1-(2-hydroxy-2-methylpropyl)piperidin-4-yl]-N-methylbenzenesulfonamide

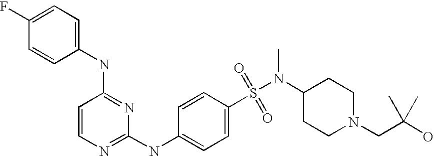

The procedure is carried out by a nucleophilic substitution reaction starting with 300 mg of 4-[4-(4-fluorophenylamino)pyrimidin-2-ylamino]-N-methyl-N-piperidin-4-ylbenzenesulfonamide (example 8) which are reacted with 81 mg of 1,2-epoxy-2-methylpropane in a microwave reactor (power: 200 W, temperature: 140° C.). 150 mg of expected product are thus obtained.

MH+=529; Melting point=216-217° C. (Trituration in isopropyl ether)

1H NMR (DMSO): 0.98 (s, 6); 1.14 (m, 2); 1.53 (ma, 2); 1.92-2.22 (m, 4); 2.61 (s, 3); 2.85 (m, 2); 3.55 (m, 1); 3.92 (s, 1); 6.23 (d, 1); 7.12 (t, 2); 7.58 (d, 2); 7.64 (m, 2); 7.99 (d, 2); 8.03 (d, 1); 9.44 (s, 1), 9.62 (s, 1).

### Example 50

### N-(2-Aminoethyl)-4-[4-(3-chloro-4-fluoro-phenylamino)pyrimidin-2-ylamino]-N-piperidin-4-yl-benzenesulfonamide hydrochloride

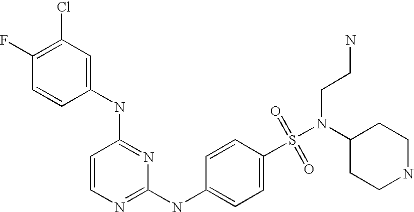

The procedure is carried out as in stage 4 of example 1 starting with 400 mg of 4-[4-(3-chloro-4-fluoro-phenylamino)pyrimidin-2-ylamino]benzenesulfonyl chloride hydrochloride (product obtained in stage 3 of example 12) which are reacted with 264 mg of 4-(2-pyrrolidin-1-ylethylamino)piperidine-1-carboxylic acid tert-butyl ester.

115 mg of expected product are thus obtained after a decarboxylation reaction according to procedure 2 of example 8.

MH+=520

Retention time (RP-HPLC, ESI, 220 nm): 4.66 minutes

### Example 51

### N-(2-Aminoethyl)-4-[4-(3,4-fluorophenyl-amino)pyrimidin-2-ylamino]-N-piperidin-4-ylbenzene-sulfonamide hydrochloride

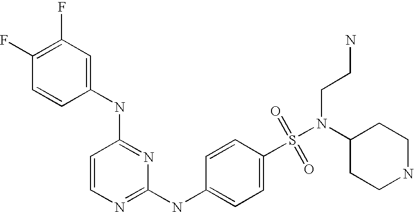

The procedure is carried out as in stage 4 of example 1 starting with 400 mg of 4-[4-(3,4-fluorophenylamino)-pyrimidin-2-ylamino]benzenesulfonyl chloride hydro-chloride (product obtained in stage 3 of example 11) which are reacted with 300 mg of methyl[2-(1-methyl-piperidin-4-ylamino)ethyl]carbamic acid tert-butyl ester.

219 mg of expected product are thus obtained after a decarboxylation reaction according to procedure 2 of example 8.

MH+=504

Retention time (RP-HPLC, ESI, 220 nm): 4.46 minutes

### Example 52

### N-(2-Aminoethyl)-4-[4-(2,4,5-fluorophenyl-amino)pyrimidin-2-ylamino]-N-piperidin-4-ylbenzene-sulfonamide hydrochloride

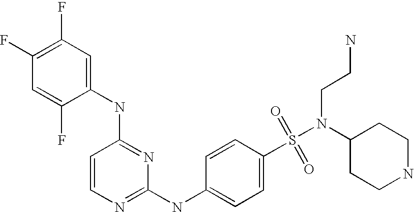

The procedure is carried out as in stage 4 of example 1 starting with 1 g of 4-[4-(2,4,5-fluorophenylamino)-pyrimidin-2-ylamino]benzenesulfonyl hydrochloride which is reacted with 828 mg of methyl[2-(1-methylpiperidin-4-ylamino)ethyl]carbamic acid tert-butyl ester.

45 mg of expected product are thus obtained after a decarboxylation reaction according to procedure 2 of example 8.

MH+=522

Retention time (RP-HPLC, ESI, 220 nm): 4.56 minutes

### Example 53

### Diethyl {2-[4-({[4-({4-[(3,4-difluoro-phenyl)amino]pyrimidin-2-yl}amino)phenyl]sulfonyl}}-amino)piperidin-1-yl]ethyl}phosphonate

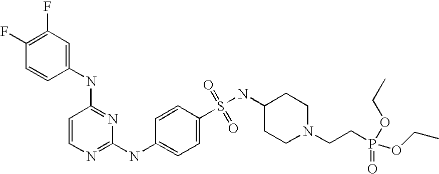

Stage 1: Diethyl [(4-tert-butoxycarboxylaminopiperidin-1-yl)ethyl]phosphonate: A mixture containing 4 g of piperidin-4-ylcarbamic acid tert-butyl ester, 5.38 g of diethyl 2-bromoethylphosphonate, 3.2 g of sodium carbonate in 50 ml of ethanol is heated under reflux for 18 hours. After cooling the reaction medium, the solid is filtered and the filtrate is concentrated under vacuum. After purification on a silica column (dichloromethane-methanol: 90-10), 6.6 g of diethyl [(4-tert-butoxycarboxylaminopiperidin-1-yl)ethyl]phosphonate with a yield of 91%.

Stage 2: Diethyl [(4-aminopiperidin-1-yl)ethyl]phosphonate: According to procedure 2 of example 8, a decarboxylation reaction, starting with 6.6 g of diethyl [(4-tert-butoxycarboxylaminopiperidin-1-yl)-ethyl]phosphonate synthesized at stage 1 makes it possible to obtain 3.7 g of expected product.

Stage 3: The procedure is carried out as in stage 4 of example 1 starting with 500 mg of 4-[4-(3,4-difluorophenylamino)pyrimidin-2-ylamino]benzenesulfonyl chloride hydrochloride and 400 mg of diethyl [(4-aminopiperidin-1-yl)ethyl]phosphonate; 480 mg of expected product are obtained with a yield of 62%.

MH+=625; Melting point: 133° C. (Isopropyl ether)

1H NMR (DMSO): 1.09 to 1.60 (unresolved complex, 10); 1.71 to 1.94 (unresolved complex, 4); 2.38 (m, 2); 2.66 (d, 2); 3.22 (m, 1); 3.94 (q, 4); 6.30 (d, 1); 7.21 to 7.52 (unresolved complex, 3); 7.67 (d, 2); 7.91 (d, 2); 8.02 to 8.17 (unresolved complex, 2); 9.66 (bs, 1); 9.72 (bs, 1).

### Example 54

### Diethyl ({4-[{[4({4-[(3,4-difluorophenyl)-amino]pyrimidin-2-yl}amino)phenyl]sulfonyl}(methyl)-amino]piperidin-1-yl}methyl)phosphonate

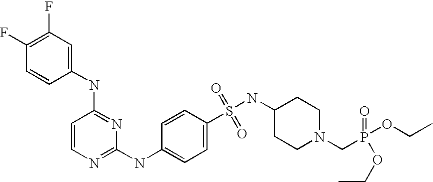

Stage 1—Diethyl [(4-tert-butoxycarboxylaminopiperidin-1-yl)methyl]phosphonate: To a solution containing 4 g of piperidin-4-ylcarbamic acid tert-butyl ester in 5 ml of dioxane are successively added 5.3 ml of a 37% aqueous formaldehyde solution and then 8.75 ml of diethyl phosphate. The reaction medium is heated under reflux for 30 minutes. After concentrating under vacuum and purifying on a silica column (dichloromethane-methanol: 90-10), 4.5 g of diethyl [(4-tert-butoxy-carboxylaminopiperidin-1-yl)methyl]phosphonate are obtained with a yield of 65%.

### Stage 2—Diethyl [(4-aminopiperidin-1-yl)methyl]phosphonate dihydrochloride

Following procedure 2 of example 8, a decarboxylation reaction, starting with 4.5 g of compound obtained in stage 1, makes it possible to obtain 4 g of expected product with a yield of 96%.

Stage 3: The procedure is carried out as in stage 4 of example 1 starting with 500 mg of 4-[4-(3,4-difluoro-phenylamino)pyrimidin-2-ylamino]benzenesulfonyl chloride hydrochloride which are reacted with 412 mg of diethyl [(4-aminopiperidin-1-yl)methyl]phosphonate dihydrochloride. 150 mg of expected product are thus obtained with a yield of 23%.

MH+=611; Melting point: 137.8° C. (Isopropyl ether)

1H NMR (DMSO): 1.09 to 1.61 (unresolved complex, 10); 2.06 (t, 2); 2.67 (d, 2); 2.76 to 2.97 (unresolved complex, 3); 3.97 (q, 4); 6.30 (d, 1); 7.20 to 7.52 (unresolved complex, 3); 7.68 (d, 2); 7.91 (d, 2); 8.01 to 8.18 (unresolved complex, 2); 9.67 (bs, 1); 9.72 (bs, 1).

### Example 55

### Diethyl (2-{4-[{[4({4-[(3,4-difluorophenyl)-amino]pyrimidin-2-yl}amino)phenyl]sulfonyl}(methyl)-amino]piperidin-1-yl}ethyl)phosphonate

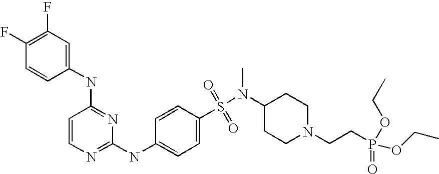

Stage 1: 4-(Benzyloxycarbonylmethylamino)piperidine-1-carboxylic acid tert-butyl ester: To a solution of 5 g of 4-methylaminopeperidin-1-carboxylic acid tert-butyl ester and 3.9 ml of triethylamine in 25 ml of dichloromethane are added dropwise, at 0° C., 4 ml of benzyl chloroformate. After stirring for 90 minutes at room temperature, 100 ml of water are added and then the medium is extracted with twice 100 ml of dichloromethane. The organic phase is dried over sodium sulfate and concentrated under vacuum. Purification by chromatography on silica (dichloromethane-ethyl acetate 95-5) makes it possible to obtain 6 g of expected product with a yield of 73%.

### Stage 2: 4-(Benzyloxycarbonylmethylamino)-1-H-piperidine hydrochloride

Following procedure 2 of example 8, a decarboxylation reaction, starting with 6.6 g of compound obtained in stage 1, 4.9 g of expected product are obtained.

Stage 3: Diethyl ({2-[4-(benzyloxycarbonylmethylamino)-piperidin-1-yl}ethyl)phosphonate: Following the process described in stage 1 of example 53, starting with 4.9 g of compound synthesized in stage 1, in the presence of 4.6 g of diethyl 2-bromoethylphosphonate and 4.5 g of sodium carbonate, 6.4 g of expected product are obtained.

Stage 4: Diethyl {2-[4-(methylamino)piperidin-1-yl)-ethyl]}phosphonate: A mixture containing 6.4 g of diethyl ({2-[4-(benzyloxycarbonylmethylamino)piperidin-1-yl}ethyl)phosphonate, 1.5 ml of cyclohexene and 210 mg of palladium hydroxide in 60 ml of ethanol is heated to reflux temperature. After 4 hours of reaction, the reaction medium is filtered on celite and concentrated under vacuum. After purifying by chromatography on silica (dichloromethane-methanol: 95-5), 800 mg of desired compound are obtained.

Stage 5: The procedure is carried out as in stage 4 of example 1 starting with 500 mg of 4-[4-(3,4-difluorophenylamino)pyrimidin-2-ylamino]benzenesulfonyl chloride hydrochloride which are reacted with 420 mg of diethyl {2-[4-(methylamino)piperidin-1-yl)ethyl]}phosphonate. 450 mg of expected product are thus obtained with a yield of 56%.

MH+=639; Melting point: 93-96° C. (Isopropyl ether)

1H NMR (DMSO): 1.11 to 1.31 (unresolved complex, 8); 1.53 (q, 2); 1.73 to 2.00 (unresolved complex, 4); 2.42 (m, 2); 2.64 (s, 3); 2.80 (d, 2); 3.60 (m, 1); 3.95 (q, 4); 6.31 (d, 1); 7.20 to 7.47 (unresolved complex, 2); 7.66 (d, 2); 7.94 (d, 2); 8.03 to 8.20 (unresolved complex, 2); 9.68 (bs, 1); 9.77 (bs, 1).

### Example 56

### Diethyl (2-{4-[(2-aminoethyl){[4-({4-[(3,4-difluorophenyl)amino]pyrimidin-2-yl}amino)phenyl]-sulfonyl}amino]piperidin-1-yl}ethyl)phosphonate

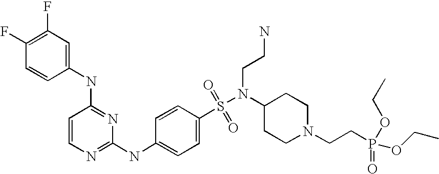

Stage 1: Diethyl [2-(4-oxopiperidin-1-yl)ethyl]phosphonate: Following the process described in stage 1 of example 53 starting with 10 g of 4-piperidone hydrochloride monohydrate and 15.7 g of diethyl 2-bromoethylphosphonate, 10 g of expected phosphonate are obtained.

Stage 2: Diethyl (2-{4-[2-(benzyloxycarbonylamino-ethyl)amino]piperidin-1-yl}ethyl)phosphonate: Following the procedure described in stage 1 of example 55, starting with 2 g of diethyl [2-(4-oxo-piperidin-1-yl)ethyl]phosphonate and 2.1 g of (2-aminoethyl)carbamic acid benzyl ester hydrochloride, 3.3 g of expected compound are obtained.

Stage 3: The procedure is carried out as in stage 4 of example 1 starting with 800 mg of 4-[4-(3,4-difluoro-phenylamino)pyrimidin-2-ylamino]benzenesulfonyl chloride hydrochloride which are reacted with 900 mg of diethyl (2-{4-[2-(benzyloxycarbonylaminoethyl)amino]-piperidin-1-yl}ethyl)phosphonate, 600 mg of a compound are obtained, which compound undergoes a hydrogenolysis reaction according to the process described in stage 4 of example 55. 170 mg of expected diethyl (2-{4-[(2-aminoethyl){[4-({4-[(3,4-difluorophenyl)amino]pyrimidin-2-yl}amino)phenyl]sulfonyl}amino]piperidin-1-yl}-ethyl)phosphonate are thus obtained.

MH+=668; Melting point: 88-90° C. (Isopropyl ether)

1H NMR (DMSO): 1.08 to 1.40 (unresolved complex, 10); 1.55 (q, 2); 1.71 to −2.00 (unresolved complex, 6); 2.42 (m, 2); 2.64 (s, 2); 2.80 (d, 2); 3.02 (t, 2); 3.49 (m, 1); 3.95 (q, 4); 6.31 (d, 1); 7.22 to 7.47 (unresolved complex, 2); 7.68 (d, 2); 7.93 (d, 2); 8.03 to 8.20 (unresolved complex, 2); 9.68 (bs, 1); 9.76 (bs, 1).

### Example 57

### Diethyl (2-{4-[(3-aminopropyl){[4-({4-[(3,4-difluorophenyl)amino]pyrimidin-2-yl}amino)-phenyl]sulfonyl}amino]piperidin-1-yl}ethyl)phosphonate

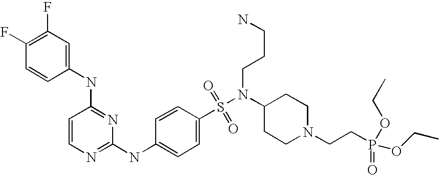

Stage 1: Diethyl (2-{4-[3-(benzyloxycarbonylamino-propyl)amino]piperidin-1-yl}ethyl)phosphonate: The procedure is carried out as in stage 1 of example 55 starting with 2 g of diethyl [2-(4-oxopiperidin-1-yl)ethyl]phosphonate and 2.2 g of (3-aminopropyl)carbamic acid benzyl ester hydrochloride.

3.4 g of expected product are obtained.

Stage 2: The procedure is carried out as in stage 4 of example 1 starting with 800 mg of 4-[4-(3,4-difluoro-phenylamino)pyrimidin-2-ylamino]benzenesulfonyl chloride hydrochloride which are reacted with 930 mg of diethyl (2-{4-[3-(benzyloxycarbonylaminopropyl)amino]-piperidin-1-yl}ethyl)phosphonate, 330 mg of a compound are obtained, which compound undergoes a hydrogenolysis reaction according to the process described in stage 4 of example 55 in order to give 300 mg of expected diethyl (2-{4-[(3-aminopropyl){[4-{4-[(3,4-difluoro-phenyl)amino]pyrimidin-2-yl}amino)phenyl]sulfonyl}-amino]piperidin-1-yl}ethyl)phosphonate.

MH+=682; Melting point: 94-96° C. (Isopropyl ether)

1H NMR (DMSO): 1.07 to 1.43 (unresolved complex, 8); 1.44 to 2.00 (unresolved complex, 8); 2.41 (t, 2); 2.56 (t, 2); 2.81 (d, 2); 3.13 (t, 2); 3.50 (m, 1); 3.95 (q, 4); 6.32 (d, 1); 7.23 to 7.47 (unresolved complex, 2); 7.68 (d, 2); 7.93 (d, 2); 8.08 to 8.20 (unresolved complex, 2); 9.69 (bs, 1); 9.75 (bs, 1).

### Example 58

### Diethyl (2-{4-[{[4-({4-[(4-fluorophenyl)-amino]pyrimidin-2-yl}amino)phenyl]sulfonyl}(methyl)-amino]piperidin-1-yl}ethyl)phosphonate

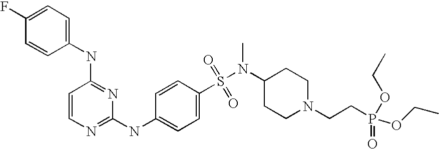

The procedure is carried out as in stage 4 of example 1 starting with 450 mg of 4-[4-(4-fluorophenylamino)-pyrimidin-2-ylamino]benzenesulfonyl chloride hydro-chloride which are reacted with 400 mg of diethyl {2-[4-(methylamino)piperidin-1-yl)ethyl]}phosphonate. 400 mg of expected product are thus obtained with a yield of 54%.

MH+=621; Melting point: 95-100° C. (Isopropyl ether)

1H NMR (DMSO): 1.07 to 1.32 (unresolved complex, 8); 1.52 (q, 2); 1.71 to 2.02 (unresolved complex, 4); 2.41 (m, 2); 2.64 (s, 2); 2.80 (d, 2); 3.60 (m, 1); 3.95 (q, 4); 6.28 (d, 1); 7.17 (triplet, 2); 7.55 to 7.79 (unresolved complex, 4); 7.95 (d, 2); 8.08 (d, 1); 9.48 (bs, 1); 9.67 (bs, 1).

### Example 59

### Diethyl (2-{4-[(2-aminoethyl){[4-({4-[(4-fluorophenyl)amino]pyrimidin-2-yl}amino)phenyl]-sulfonyl}amino]piperidin-1-yl}ethyl)phosphonate

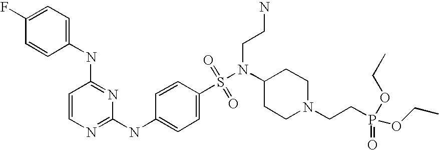

The procedure is carried out as in stage 4 of example 1 starting with 1 g of 4-[4-(4-fluorophenylamino)pyrimidin-2-ylamino]benzenesulfonyl chloride hydrochloride which is reacted with 1.39 g of diethyl (2-{4-[2-(benzyloxycarbonylaminoethyl)amino]piperidin-1-yl}-ethyl)phosphonate (stage 2 example 56). 688 mg of a compound are thus obtained, which compound undergoes a hydrogenolysis reaction according to the process described in stage 4 of example 55 in order to give 150 mg of expected product with a yield of 26%.

MH+=650; Melting point: 90° C. (Isopropyl ether)

1H NMR (DMSO): 1.09 to 1.40 (unresolved complex, 8); 1.55 (q, 2); 1.72 to 2.10 (unresolved complex, 4); 2.41 (m, 2); 2.64 (t, 2); 2.81 (d, 2); 3.02 (t, 2); 3.50 (m, 1); 3.95 (qui, 4); 6.28 (d, 1); 7.17 (triplet, 2); 7.58 to 7.78 (unresolved complex, 4); 7.93 (d, 2); 8.08 (d, 1); 8.13 to 8.20 (unresolved complex, 2); 9.49 (bs, 1); 9.66 (bs, 1).

### Example 60

### Diethyl {2-[4-({[4-({4-[(4-fluorophenyl)-amino]pyrimidin-2-yl}amino)phenyl]sulfonyl}}amino)-piperidin-1-yl]propyl}phosphonate

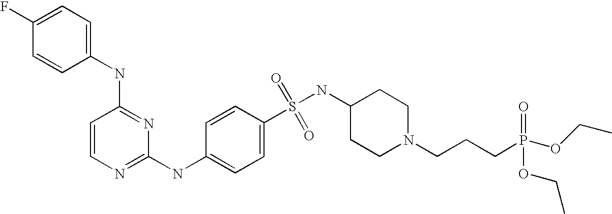

Stage 1: [4-(4-Aminopiperidin-1-yl)butyl]phosphonic acid diethyl ester: Following the procedure described in stage 1 of example 53, starting with 5 g of piperidin-4-ylcarbamic acid tert-butyl ester in the presence of 7.4 g of (4-bromobutyl)phosphonic acid diethyl dihydrochloride, a compound is obtained which undergoes a decarboxylation reaction according to procedure 2 of example 8 in order to give 6.9 g of expected product.

Stage 2: Following the process described in stage 4 of example 1 starting with 800 mg of 4-[4-(4-fluorophenyl-amino)pyrimidin-2-ylamino]benzenesulfonyl chloride hydrochloride and 670 mg of (4-bromobutyl)phosphonic acid diethyl ester dihydrochloride, 6.9 g of [4-(4-aminopiperidin-1-yl)butyl]phosphonic acid diethyl ester are obtained. 400 mg of expected product are obtained after chromatography on silica (dichloromethane methanol 88/12).

MH+=621.1; Melting point: 110° C. (Isopropyl ether)

1H NMR (DMSO): 1.14 (t, 6); 1.19 to 1.87 (unresolved complex, 10); 2.16 (m, 2); 2.59 (m, 2); 2.82 (m, 1); 3.89 (m, 4); 6.23 (d, 1); 7.13 (t, 2); 7.40 (d, 1); 7.56 to 7.71 (unresolved complex, 4); 7.86 (d, 2); 8.03 (d, 1); 9.43 (s, 1); 9.58 (s, 1).

### Example 61

### Diethyl (2-{4-[{[4-({4-[(3-methyl-4-fluoro-phenyl)amino]pyrimidin-2-yl}amino)phenyl]sulfonyl}-(methyl)amino]piperidin-1-yl}ethyl)phosphonate

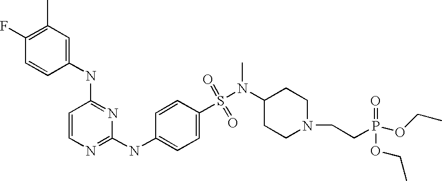

Stage 1: [2-(4-Methylaminopiperidin-1-yl)ethyl]phosphonic acid diethyl ester: Following the procedure described in stage 2 of example 56, starting with 1 g of compound diethyl [2-(4-oxo-piperidin-1-yl)ethyl]phosphonate obtained in stage 1 of example 56 in the presence of 2.3 ml of a 2N solution of methylamine in THF, 800 mg of are obtained.

Stage 2: The procedure is carried out as in stage 4 of example 1 starting with 600 mg of 4-[4-(3-methyl-4-fluorophenylamino)pyrimidin-2-ylamino]benzenesulfonyl chloride hydrochloride and 510 mg of [2-(4-methylamino-piperidin-1-yl)ethyl]phosphonic acid diethyl ester, 640 mg of expected product are obtained.

MH+=635.2

1H NMR (DMSO): 1.31 (m, 8); 1.55 (m, 2); 1.99 (m, 4); 2.35 (s, 3); 2.52 (m, 2); 2.76 (s, 3); 2.92 (m, 2); 3.73 (m, 1); 4.98 (q, 4); 6.40 (d, 1); 7.22 (t, 1); 7.57 (m, 1); 7.69 (m, 1); 7.74 (d, 2); 8.07 (d, 2); 8.20 (d, 1); 9.53 (s, 1); 9.79 (s, 1).

### Example 62

### Diethyl (2-{4-[(pyrrolidin-2-R-ylmethyl){[4-({4-[(4-fluorophenyl)amino]pyrimidin-2-yl}amino)-phenyl]sulfonyl}amino]piperidin-1-yl}ethyl)phosphonate

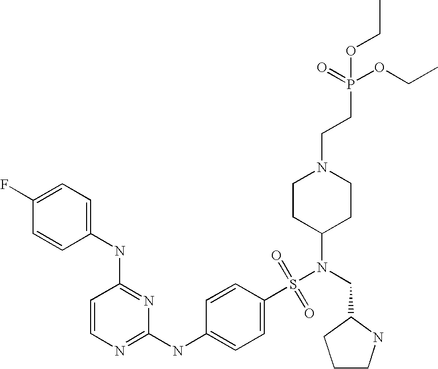

Stage 1: (2-{4-[(Pyrrolidin-2-R-ylmethyl)amino]piperidin-1-ylcarbamic acid tert-butyl ester}ethyl)phosphonic acid diethyl ester: As in example A, starting with 2 g of compound diethyl [2-(4-oxopiperidin-1-yl)ethyl]phosphonate obtained in stage 1 of example 56 in the presence of 1.8 g of pyrrolidin-2-R-ylmethylamine, 2.8 g of expected compound are obtained, [α] (20,589).=+39° (C=0.1, MeOH).

Stage 2: The procedure is carried out as in stage 4 of example 1 starting with 800 mg of 4-[4-(3-methyl-4-fluorophenylamino)pyrimidin-2-ylamino]benzenesulfonyl chloride hydrochloride and 1.13 g of (2-{4-[(pyrrolidin-2-R-ylmethyl)amino]piperidin-1-ylcarbamic acid tert-butyl ester}ethyl)phosphonic acid diethyl ester, 700 mg of a compound are obtained which are treated by a decarboxylation reaction according to procedure 2 of example 8 in order to give 550 of expected product in hydrochloride form.

MH+=690.2; Melting point 185° C. (Isopropyl ether)

1H NMR (DMSO): 1.38 (t, 6); 1.50 to 2.6 (unresolved complex, 10); 2.96 to 4.13 (unresolved complex, 16); 6.55 (d, 1); 7.39 (t, 2); 7.81 (m, 2); 8.0 (m, 4); 8.26 (d, 1); 9.0 (bs, 1); 9.57 (bs, 1); 10.67 (ml, 2); 11.19 (ml, 1).

### Example 63

### Diethyl (2-{4-[(pyrrolidin-2-S-ylmethyl){[4-({4-[(4-fluorophenyl)amino]pyrimidin-2-yl}amino)-phenyl]sulfonyl}amino]piperidin-1-yl}ethyl)phosphonate

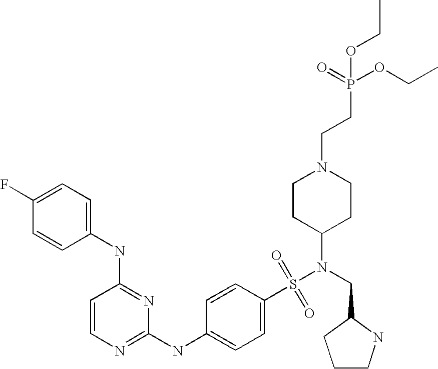

Stage 1: (2-{4-[(Pyrrolidin-2-S-ylmethyl)amino]piperidin-1-ylcarbamic acid tert-butyl ester}ethyl)phosphonic acid diethyl ester: As in example A, starting with 2 g of compound diethyl [2-(4-oxopiperidin-1-yl)ethyl]phosphonate obtained in stage 1 of example 56 and 1.8 g of pyrrolidin-2-S-ylmethylamine, 2.8 g of expected compound are obtained, [α] (20,589).=−33° (C=0.1, MeOH).

Stage 2: The procedure is carried out as in stage 4 of example 1 starting with 800 mg of 4-[4-(3-methyl-4-fluorophenylamino)pyrimidin-2-ylamino]benzenesulfonyl chloride hydrochloride and 1.13 g of (2-{4-[(pyrrolidin-2-S-ylmethyl)amino]piperidin-1-ylcarbamic acid tert-butyl ester}ethyl)phosphonic acid diethyl ester, 660 mg of a compound are obtained which are treated by a decarboxylation reaction according to procedure 2 of example 8 in order to give 490 g of expected product in hydrochloride form.

MH+=690.2; Melting point 185° C. (Isopropyl ether)

1H NMR (DMSO): 1.38 (t, 6); 1.50-2.6 (unresolved complex, 10); 2.96-4.13 (unresolved complex, 16); 6.55 (d, 1); 7.39 (t, 2); 7.81 (m, 2); 8 (m, 4); 8.26 (d, 1); 8.68 (bs, 1); 9.29 (bs, 1); 10.12 (ml, 2); 11.85 (ml, 1)

### Example 64

### Diethyl (2-{4-[(3-aminopropyl){[4({4-[(4-fluorophenyl)amino]pyrimidin-2-yl}amino)phenyl]-sulfonyl}amino]piperidin-1-yl}ethyl)phosphonate

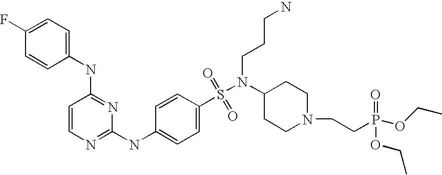

Following the process described in stage 4 of example 1 starting with 600 mg of 4-[4-(4-fluorophenylamino)-pyrimidin-2-ylamino]benzenesulfonyl chloride hydro-chloride and 510 mg of diethyl (2-{4-[3-(benzyloxy-carbonylaminopropyl)amino]piperidin-1-yl}ethyl)phosphonate, 70 mg of expected product are obtained after a hydrogenolysis reaction described in stage 4 of example 55.

MH+=664.2; Melting point: 125-130° C. (Isopropyl ether)

1H NMR (DMSO): 1.14 (t, 6); 1.21 to 2.04 (unresolved complex, 10); 2.36 (m, 4); 2.74 (m, 2); 3.06 (m, 2); 3.46 (m, 1); 3.88 (m, 4); 6.22 (d, 1); 7.11 (t, 2); 7.94 (d, 2); 7.63 (m, 2); 7.88 (d, 2); 8.05 (d, 1); 9.94 (bs, 1); 9.57 (bs, 1).

### Example 65

### Diethyl (2-{4-[(2-pyrrolidin-1-ylethyl-amino){[4-({4-[(4-fluorophenyl)amino]pyrimidin-2-yl}-amino)phenyl]sulfonyl}amino]piperidin-1-yl}ethyl)-phosphonate

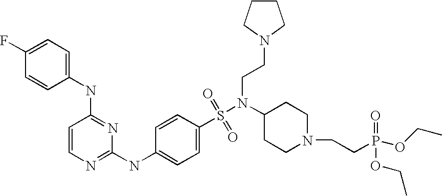

Stage 1: {2-[4-(2-Pyrrolidin-1-ylethylamino)piperidin-1-yl]ethyl}phosphonic acid diethyl ester: As in example A, starting with 2 g of compound diethyl[2-(4-oxopiperidin-1-yl)ethyl]phosphonate obtained in stage 1 of example 56 and 1 g of 2-pyrrolidin-1-ylethylamine, 2.7 g of expected compound are obtained.

Stage 2: Following the process described in stage 4 of example 1 starting with 600 mg of 4-[4-(4-fluorophenyl-amino)pyrimidin-2-ylamino]benzenesulfonyl chloride hydrochloride and 680 mg of {2-[4-(2-pyrrolidin-1-yl-ethylamino)piperidin-1-yl]ethyl}phosphonic acid diethyl ester, 340 mg of expected product are obtained.

MH+=704.3; Melting point: 85° C. (Isopropyl ether).

1H NMR (DMSO): 1.21 (t, 6); 1.37 (d, 2); 1.55 (q, 2); 1.69 (bs, 4); 1.79 to 1.97 (unresolved complex, 4); 2.38 to 2.70 (unresolved complex, 8); 2.83 (d, 2); 3.18 (t, 2); 3.52 (m, 1); 3.96 (m, 4); 6.29 (d, 1); 7.17 (t, 2); 7.63 to 7.74 (unresolved complex, 4); 7.93 (d, 2); 8.08 (d, 1); 9.47 (bs, 1); 9.65 (bs, 1).

The products of examples 66 to 85 may be prepared in particular according to synthesis scheme 1 above according to the reaction conditions indicated below.

It may be noted that the product thus obtained comprises 2 cis and trans diastereoisomers and may further comprise two enantiomers:

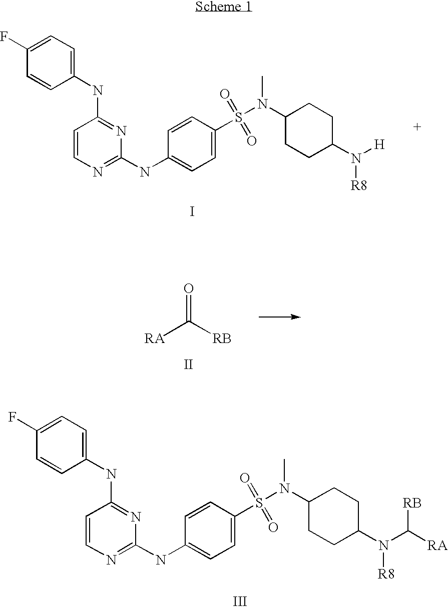

To a solution of 0.144 mmol (1.0 eq) of amine I (example 47) and 0.52 mmol (3.7 eq) of EtNiPr₂ in 7 ml of methanol is added 0.148 mmol (1.03 eq) of aldehyde II and then 0.276 mmol (2.0 eq) of a 1M solution of NaCNBH3 in THF*. The reaction medium is kept stirring at room temperature overnight. 0.148 mmol of aldehyde, 0.2 mmol of NaCNBH3 (1M in THF) and 0.525 mmol of acetic acid are added and the reaction medium is kept stirring throughout the weekend. After filtration, washing of the residue with 5 ml of methanol, the filtrate is concentrated under vacuum. The crude reaction product is with 20 ml of ethyl acetate and washed with 20 ml of a 5% Na2CO3 solution and washed with 20 ml of a saturated NaCl solution. After drying over Na2SO4 and concentrating to dryness, the crude product is purified on a preparative rp-HPLC column (MeCN/H2O/TFA gradient), and the product obtained is freeze-dried.

### Rp-HPLC:

HPLC/MS Analysis (Waters MassLynx, Binary gradient (H2O+0.05% TFA)/(CH3CN+0.05% TFA): 0.0 min, 5.0% CH3CN to 2.5 min, 95% CH3CN, flux: 1.3 ml/min, 30° C., 2×33 mm YMC J′sphere, 4μ)

### Example 66

### 4-[4-(4-Fluorophenylamino)pyrimidin-2-yl-amino]-N-methyl-N-[4-(1-pyridin-4-ylethylamino)cyclo-hexyl]benzenesulfonamide trifluoroacetic acid

The procedure is carried out as indicated above using compound II, 1-pyridin-4-ylethanone, and 6.6 mg of expected compound are obtained.

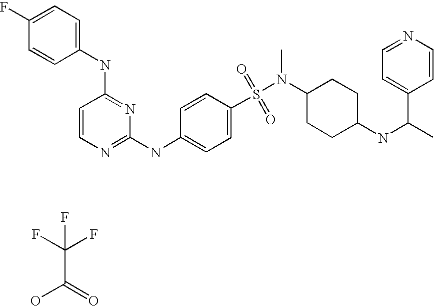

Rt=1.08 min

MH+=576.25

### Example 67

### N-{4-[(2,3-Dihydro-1H-indol-7-ylmethyl)-amino]cyclohexyl}-4-[4-(4-fluorophenylamino)pyrimidin-2-ylamino]-N-methylbenzenesulfonamide trifluoroacetic acid

The procedure is carried out as indicated above, using compound II, 2,3-dihydro-1H-indole-7-carbaldehyde, and 24.3 mg of expected compound are obtained.

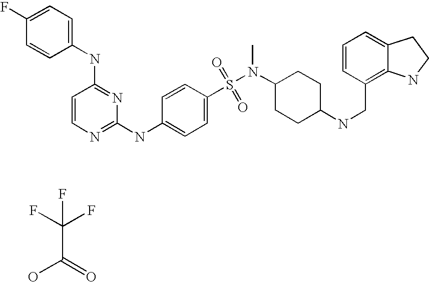

Rt=1.20 min

MH+=602.24

### Example 68

### 4-[4-(4-Fluorophenylamino)pyrimidin-2-yl-amino]-N-methyl-N-[4-(1-methyl-2-pyridin-4-ylethyl-amino)cyclohexyl]benzenesulfonamide trifluoroacetic acid

The procedure is carried out as indicated above, using compound II, 1-(pyridin-4-yl)acetone, and 36.7 mg of expected compound are obtained.

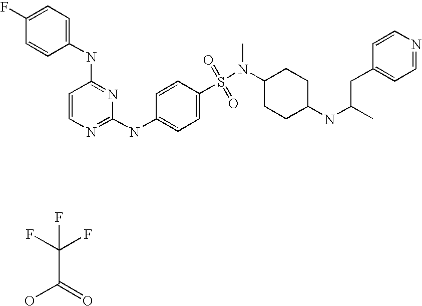

Rt=1.09 min

MH+=590.25

### Example 69

### 4-[4-(4-Fluorophenylamino)pyrimidin-2-yl-amino]-N-[4-(4-hydroxy-3-trifluoromethylbenzylamino)-cyclohexyl]-N-methylbenzenesulfonamide trifluoroacetic acid

The procedure is carried out as indicated above, using compound II, 4-hydroxy-3-(trifluoromethyl)benzaldehyde, and 37.8 mg of expected compound are obtained.

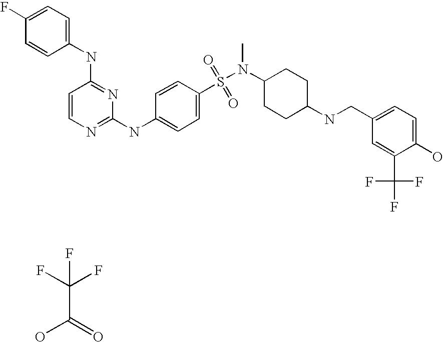

Rt=1.26 min

MH+=645.23

### Example 70

### 4-[4-(4-Fluorophenylamino)pyrimidin-2-yl-amino]-N-methyl-N-{4-[(quinolin-5-ylmethyl)amino]cyclo-hexyl}benzenesulfonamide trifluoroacetic acid

The procedure is carried out as indicated above, using compound II, quinoline-5-carboxaldehyde, and 40.4 mg of expected compound are obtained.

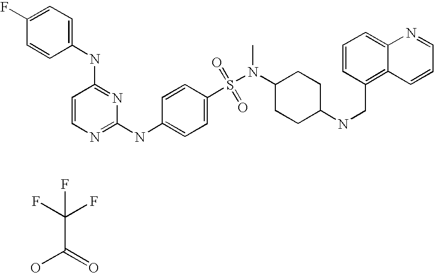

Rt=1.05 min

MH+=612.21

### Example 71

### 4-[4-(4-Fluorophenylamino)pyrimidin-2-yl-amino]-N-methyl-N-{4-[(pyrimidin-5-ylmethyl)amino]-cyclohexyl}benzenesulfonamide trifluoroacetic acid

The procedure is carried out as indicated above, using compound II, pyrimidine-5-carboxaldehyde, and 35.7 mg of expected compound are obtained.

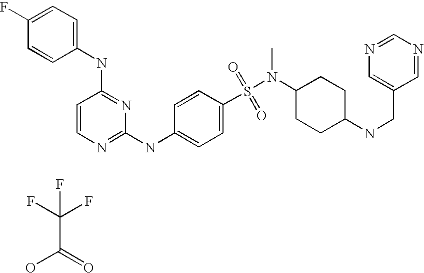

Rt=1.06 min

MH+=563.21

### Example 72

### 4-[4-(4-Fluorophenylamino)pyrimidin-2-yl-amino]-N-methyl-N-{4-[(pyridin-2-ylmethyl)amino]cyclo-hexyl}benzenesulfonamide trifluoroacetic acid

The procedure is carried out as indicated above, using compound II, pyridine-2-carboxaldehyde, and 31.6 mg of expected compound are obtained.

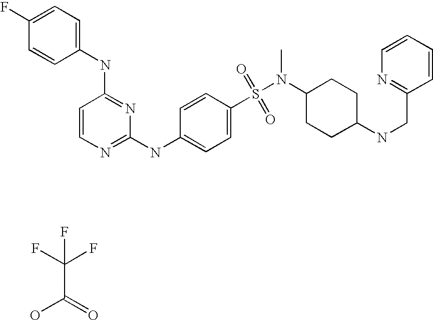

Rt=1.13 min

MH+=562.21

### Example 73

### 4-[4-(4-Fluorophenylamino)pyrimidin-2-yl-amino]-N-methyl-N-{4-[(1-methyl-1H-pyrrol-3-ylmethyl)-amino]cyclohexyl}benzenesulfonamide trifluoroacetic acid

The procedure is carried out as indicated above, using compound II, 1-methyl-1H-pyrrole-3-carboxaldehyde, and 33.1 mg of expected compound are obtained.

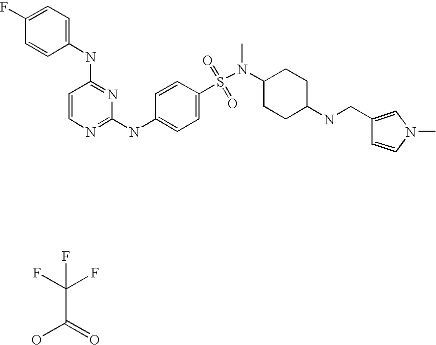

Rt=1.17 min

MH+=564.21

### Example 74

### 4-[4-(4-Fluorophenylamino)pyrimidin-2-yl-amino]-N-methyl-N-{4-[(2-methylaminopyridin-3-yl-methyl)amino]cyclohexyl}benzenesulfonamide trifluoroacetic acid

The procedure is carried out as indicated above, using compound II, 2-(methylamino)nicotinaldehyde, and 28.3 mg of expected compound are obtained.

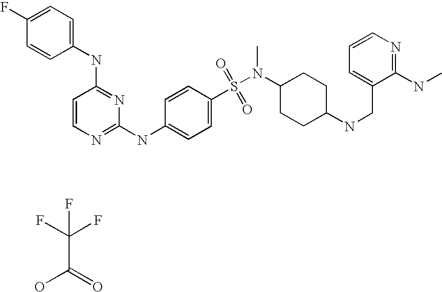

Rt=1.08 min

MH+=591.25

### Example 75

### N-{4-[(5-Cyano-1,2-dimethyl-1H-pyrrol-3-yl-methyl)amino]cyclohexyl}-4-[4-(4-fluorophenylamino)-pyrimidin-2-ylamino]-N-methylbenzenesulfonamide trifluoroacetic acid

The procedure is carried out as indicated above, using compound II, 5-formyl-1,5-dimethyl-1H-pyrrole-2-carbox-aldehyde, and 32.2 mg of expected compound are obtained.

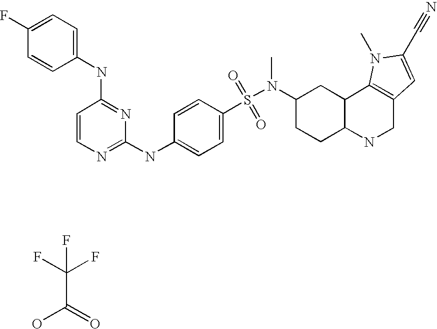

Rt=1.24 min

MH+=603.23

### Example 76

### 4-[4-(4-Fluorophenylamino)pyrimidin-2-yl-amino]-N-methyl-N-{4-[(pyridin-4-ylmethyl)amino]cyclo-hexyl}benzenesulfonamide trifluoroacetic acid

The procedure is carried out as indicated above, using compound II, isonicotinaldehyde, and 19.2 mg of expected compound are obtained.

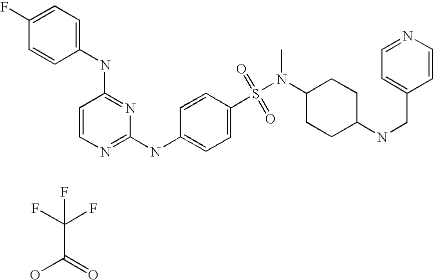

Rt=1.08 min

MH+=562.21

### Example 77

### 4-[4-(4-Fluorophenylamino)pyrimidin-2-yl-amino]-N-methyl-N-[4-(1-pyridin-2-ylethylamino)cyclo-hexyl]benzenesulfonamide trifluoroacetic acid

The procedure is carried out as indicated above, using compound II, 1-pyridin-2-ylethanone, and 16.1 mg of expected compound are obtained.

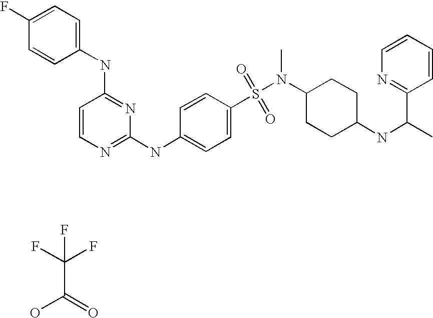

Rt=1.20 min

MH+=576.22

### Example 78

### N-{4-[(2,3-Dihydrobenzofuran-5-ylmethyl)-amino]cyclohexyl}-4-[4-(4-fluorophenylamino)pyrimidin-2-ylamino]-N-methylbenzenesulfonamide trifluoroacetic acid

The procedure is carried out as indicated above, using compound II, 2,3-dihydro-1-benzofuran-5-cardaldehyde, and 26.8 mg of expected compound are obtained.

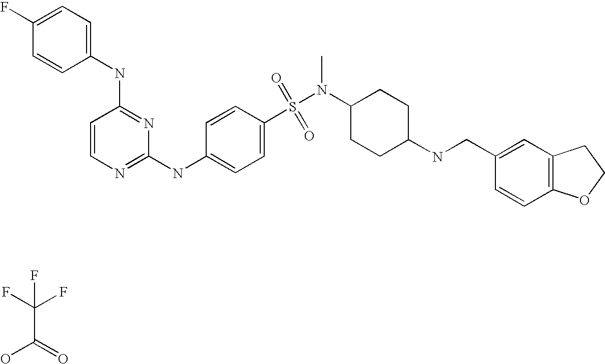

Rt=1.23 min

MH+=603.22

### Example 79

### 4-[4-(4-Fluorophenylamino)pyrimidin-2-yl-amino]-N-methyl-N-{4-[(pyridin-3-ylmethyl)amino]cyclo-hexyl}benzenesulfonamide trifluoroacetic acid

The procedure is carried out as indicated above, using compound II, nicotinaldehyde, and 40.2 mg of expected compound are obtained.

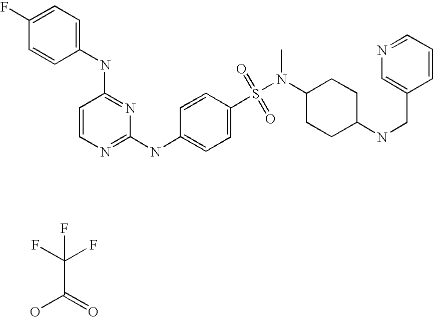

Rt=1.08 min

MH+=562.22

### Example 80

### 4-[4-(4-Fluorophenylamino)pyrimidin-2-yl-amino]-N-methyl-N-{4-[(quinolin-6-ylmethyl)amino]cyclo-hexyl}benzenesulfonamide trifluoroacetic acid

The procedure is carried out as indicated above, using compound II, quinoline-6-carboxaldehyde, and 45.5 mg of expected compound are obtained.

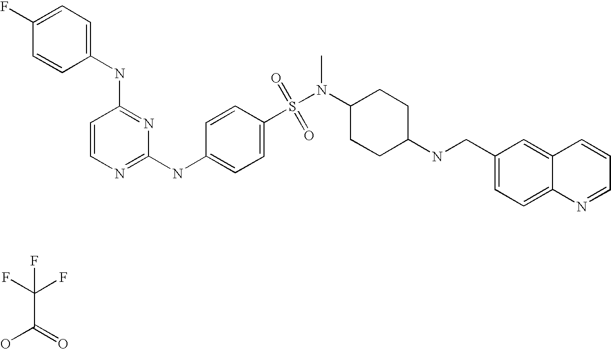

Rt=1.06 min

MH+=612.24

### Example 81

### N-{4-[(2-Aminopyridin-3-ylmethyl)amino]-cyclohexyl}-4-[4-(4-fluorophenylamino)pyrimidin-2-yl-amino]-N-methylbenzenesulfonamide trifluoroacetic acid

The procedure is carried out as indicated above, using compound II, 2-aminonicotinaldehyde, and 43.6 mg of expected compound are obtained.

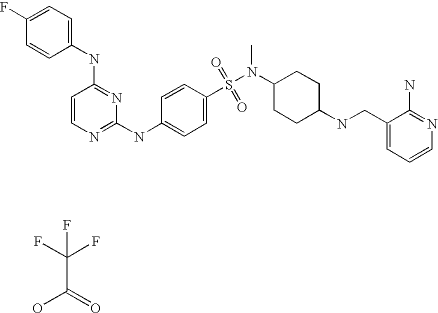

Rt=1.07 min

MH+=577.23

### Example 82

### 4-[4-(4-Fluorophenylamino)pyrimidin-2-yl-amino]-N-{4-[(isoquinolin-4-ylmethyl)amino]cyclohexyl}-N-methylbenzenesulfonamide trifluoroacetic acid

The procedure is carried out as indicated above, using compound II, isoquinoline-4-carbaldehyde, and 42.2 mg of expected compound are obtained.

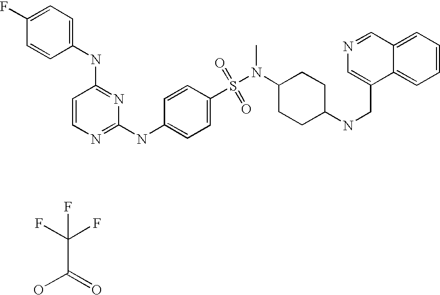

Rt=1.15 min

MH+=612.22

### Example 83

### 4-[4-(4-Fluorophenylamino)pyrimidin-2-yl-amino]-N-methyl-N-{4-[([1,8]naphthyridin-2-ylmethyl)-amino]cyclohexyl}benzenesulfonamide trifluoroacetic acid

The procedure is carried out as indicated above, using compound II, 1,8-naphthyridine-2-carboxaldehyde, and 17.4 mg of expected compound are obtained.

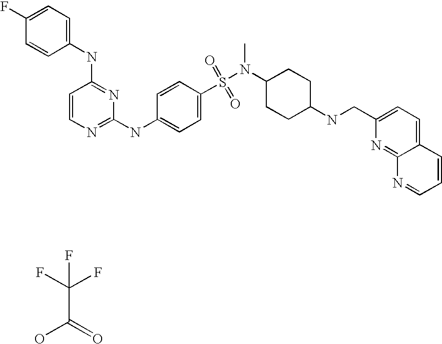

Rt=1.12 min

MH+=613.26

### Example 84

### N-{4-[(Benzo[1,2,5]oxadiazol-5-ylmethyl)-amino]cyclohexyl}-4-[4-(4-fluorophenylamino)pyrimidin-2-ylamino]-N-methylbenzenesulfonamide trifluoroacetic acid

The procedure is carried out as indicated above, using compound II, 2,1,3-benzoxadiazole-5-carboxaldehyde, and 22.8 mg of expected compound are obtained.

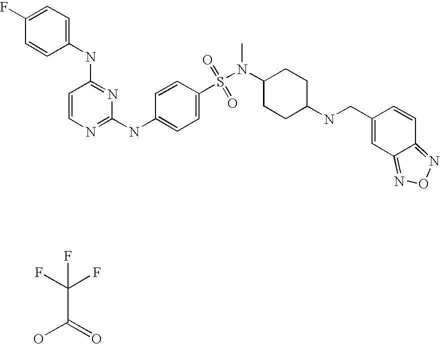

Rt=1.27 min

MH+=603.21

### Example 85

### N-{4-[(2,3-Dihydrobenzofuran-7-ylmethyl)-amino]cyclohexyl}-4-[4-(4-fluorophenylamino)pyrimidin-2-ylamino]-N-methylbenzenesulfonamide trifluoroacetic acid

The procedure is carried out as indicated above, using compound II, 2,3-dihydro-1-benzofuran-6-carboxaldehyde, and 23.1 mg of expected compound are obtained.

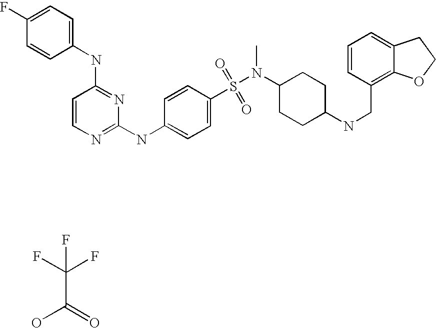

Rt=1.28 min

MH+=603.21

### Example 86

### 4-({4-[(4-fluoro-3-methylphenyl)amino]-pyrimidin-2-yl}amino)-N-methyl-N-(1-methylpiperidin-4-yl)benzenesulfonamide

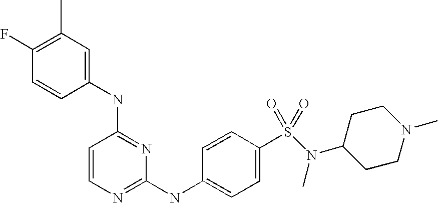

The procedure is carried out as in stage 4 of example 1, starting with 450 mg of 4-({4-[(4-fluoro-3-methyl-phenyl)amino]pyrimidin-2-yl}amino)benzenesulfonyl chloride hydrochloride which are reacted with 170 mg of methyl-(1-methylpiperidin-4-yl)amine. 181 mg of expected product are obtained.

MH+=485.0; Melting point: 110-120° C. (Isopropyl ether/dichloromethane)

### Example 87

### 4-({4-[(4-Fluoro-3-methylphenyl)amino]-pyrimidin-2-yl}amino)-N-(1-methylpiperidin-4-yl)-N-(2-pyrrolidin-1-ylethyl)benzenesulfonamide

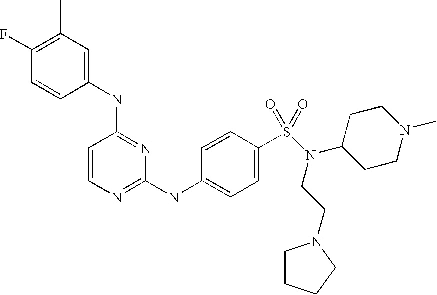

Stage 1: (1-Methylpiperidin-4-yl)(2-pyrrolidin-1-yl-ethyl)amine: As in example A, starting with 3 ml of 1-methylpiperidin-4-one and 3.35 ml of 2-pyrrolidin-1-ylethylamine, 4.4 g of expected product are obtained.

Stage 2: (1-Methylpiperidin-4-yl)(2-pyrrolidin-1-yl-ethyl)carbamic acid tert-butyl ester: A mixture containing 4.4 g of the compound obtained in stage 1 is dissolved in 100 ml of dichloromethane. 4.7 g of Boc2O are added to the reaction medium and the medium is heated at 50° C. for 1 h 30 min. After concentrating to dryness, the crude product is purified on an alumina column (dichloromethane in a gradient up to 2% methanol). 2.35 g of expected compound are obtained in total.

Stage 3: (1-Methylpiperidin-4-yl)(2-pyrrolidin-1-yl-ethyl)amine hydrochloride: Starting with 1.85 g of product obtained in stage 2, 1.65 g of expected product are obtained after a decarboxylation reaction following the procedure of example 8.

Stage 4: 4-({4-[(4-Fluoro-3-methylphenyl)amino]pyrimidin-2-yl}amino)-N-(1-methylpiperidin-4-yl)-N-(2-pyrrolidin-1-ylethyl)benzenesulfonamide: The procedure is carried out as in stage 4 of example 1, starting with 390 mg of 4-({4-[(4-fluoro-3-methylphenyl)amino]pyrimidin-2-yl}amino)benzenesulfonyl chloride hydrochloride and 300 mg of (1-methylpiperidin-4-yl)(2-pyrrolidin-1-ylethyl)amine hydrochloride. 145 mg of expected product are thus obtained.

MH+=568.2; Melting point: 93-103° C. (Isopropyl ether)

1H NMR (DMSO): 1.33 (d, 2); 1.58 (qd, 2); 1.62 to 1.70 (unresolved complex, 4); 1.84 (t, 2); 2.09 (s, 3); 2.25 (s, 3); 2.40 to 2.49 (unresolved complex, 4); 2.57 (t, 2); 2.72 (d, 2); 3.16 (t, 2); 3.48 (m, 1); 6.28 (d, 1); 7.10 (t, 1); 7.46 (m, 1); 7.58 (dd, 1); 7.66 (d, 2); 7.93 (d, 2); 8.08 (d, 1); 9.41 (bs, 1); 9.67 (bs, 1).

### Example 88

### 4-({4-[(4-Fluorophenyl)amino]pyrimidin-2-yl}amino)-N-(2-pyrrolidin-1-ylethyl)-N-(tetrahydro-2H-thiopyran-4-yl)benzenesulfonamide

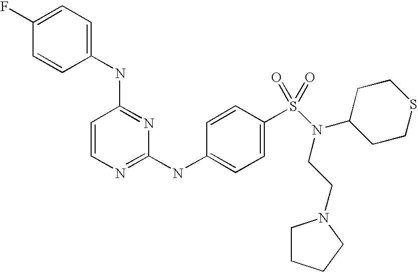

Stage 1: (2-Pyrrolidin-1-ylethyl)(tetrahydrothiopyran-4-yl)amine: As in example A, starting with 5 g of tetrahydrothiopyran-4-one and 5.90 g of 2-pyrrolidin-1-ylethylamine, 3.9 g of (2-pyrrolidin-1-ylethyl)(tetra-hydrothiopyran-4-yl)amine are obtained.

Stage 2: 4-({4-[(4-Fluorophenyl)amino]pyrimidin-2-yl}-amino)-N-(2-pyrrolidin-1-ylethyl)-N-(tetrahydro-2H-thio-pyran-4-yl)benzenesulfonamide: The procedure is carried out as in stage 4 of example 1 starting with 600 mg of 4-({4-[(4-fluorophenyl)amino]pyrimidin-2-yl}amino)-benzenesulfonyl chloride hydrochloride which are reacted with 342 mg of (2-pyrrolidin-1-ylethyl)(tetrahydrothiopyran-4-yl)amine hydrochloride. 180 mg of expected product are thus obtained.

MH+=557.2;

1H NMR (DMSO): 1.42-1.86 (unresolved complex, 8); 2.19-2.79 (unresolved complex, 10); 3.09 (m, 2); 3.55 (m, 1); 6.24 (d, 1); 7.14 (t, 2); 7.54-7.75 (unresolved complex, 4); 7.89 (d, 2); 8.04 (d, 1); 9.44 (s, 1); 9.62 (s, 1)

### Example 89

### 4-({4-[(4-Fluorophenyl)amino]pyrimidin-2-yl}amino)-N-(1-methylpiperidin-4-yl)-N-(2-pyrrolidin-1-ylethyl)benzenesulfonamide

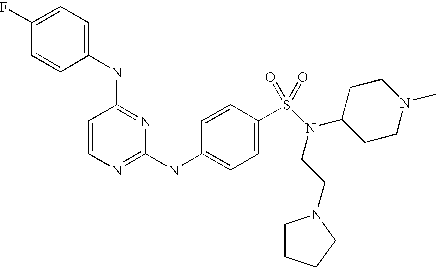

Stage 1: 4-(2-Pyrrolidin-1-ylethylamino)piperidine-1-carboxylic acid tert-butyl ester: As in example A, starting with 3 g of 4-oxopiperidine-1-carboxylic acid tert-butyl ester and 2 g of 2-pyrrolidin-1-ylethylamine, 1.5 of 4-(2-pyrrolidin-1-ylethylamino)piperidine-1-carboxylic acid tert-butyl ester are obtained.

Stage 2: 4-[{4-[4-(4-Fluorophenylamino)pyrimidin-2-yl-amino]benzenesulfonyl}-(2-pyrrolidin-1-ylethyl)amino]-piperidine-1-carboxylic acid tert-butyl ester: The procedure is carried out as in stage 4 of example 1, starting with 720 mg of 4-({4-[(4-fluorophenyl)amino]-pyrimidin-2-yl}amino)benzenesulfonyl chloride hydro-chloride which are reacted with 570 mg of 4-(2-pyrrolidin-1-ylethylamino)piperidine-1-carboxylic acid tert-butyl ester. 230 mg of expected product are thus obtained.

Stage 3: 4-[4-(4-Fluorophenylamino)pyrimidin-2-ylamino]-N-piperidin-4-yl-N-(2-pyrrolidin-1-ylethyl)-benzenesulfonamide: Starting with 230 mg of compound obtained in stage 1, 160 mg of expected product are obtained after a decarboxylation reaction following procedure 2 of example 8.

Stage 4: 4-({4-[(4-Fluorophenyl)amino]pyrimidin-2-yl}-amino)-N-(1-methylpiperidin-4-yl)-N-(2-pyrrolidin-1-yl-ethyl)benzenesulfonamide: As in example A, starting with 160 mg of product obtained in stage 3 and 0.05 ml of formaldehyde, 146 mg of expected product are obtained.

MH+=554.7; Melting point: 250° C. (Isopropyl ether)

1H NMR (DMSO): 1.58 (d, 2); 1.77 to 2.26 (m, 6); 2.66 (d, 3); 2.94 to 3.19 (unresolved complex, 4); 3.25 to 3.71 (unresolved complex, 8); 4.02 (m, 1); 6.54 (d, 1); 7.30 (t, 2); 7.67 (dd, 2); 7.86 (unresolved complex, 4); 8.12 (d, 1); 10.82 (bs, 2); 10.93 to 11.17 (unresolved complex, 2).

### Example 90

### N-(2-Aminoethyl)-N-(1-benzylpiperidin-4-yl)-4-({4-[(4-fluorophenyl)amino]pyrimidin-2-yl}amino)-benzenesulfonamide hydrochloride

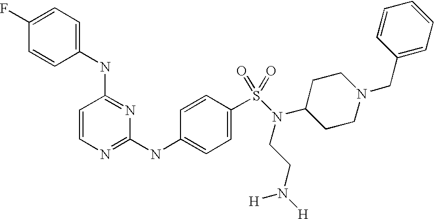

Stage 1: [2-(1-Benzylpiperidin-4-ylamino)ethyl]carbamic acid tert-butyl ester: As in example A, starting with 4 g of 4-benzylpiperidone and 3.4 g of (2-aminoethyl)carbamic acid tert-butyl ester, 3.2 g of [2-(1-benzylpiperidin-4-ylamino)ethyl]carbamic acid tert-butyl ester are obtained.

Stage 2: [2-((1-Benzylpiperidin-4-yl)-{4-[4-(4-fluoro-phenylamino)pyrimidin-2-ylamino]benzenesulfonyl}amino)-ethyl]carbamic acid tert-butyl ester: The procedure is carried out as in stage 4 of example 1, starting with 2.7 g of 4-[4-(4-fluorophenylamino)pyrimidin-2-yl-amino]benzenesulfonyl chloride hydrochloride and 3 g of [2-(1-benzylpiperidin-4-ylamino)ethyl]carbamic acid tert-butyl ester in the presence of triethylamine. 1 g of expected product is thus obtained.

Stage 3: N-(2-Aminoethyl)-N-(1-benzylpiperidin-4-yl)-4-({4-[(4-fluorophenyl)amino]pyrimidin-2-yl}amino)-benzenesulfonamide hydrochloride: Starting with 640 mg of product obtained in stage 2, 630 mg of expected product are obtained after a decarboxylation reaction following procedure 2 of example 8.

MH+=576.3; Melting point: 230-232° C. (Isopropyl ether)

1H NMR (DMSO): 1.34 to 1.76 (unresolved complex, 2); 2 to 2.4 (unresolved complex, 2); 3.03 (m, 4); 3.31 (m, 41); 3.65 to 4.16 (unresolved complex, 1); 4.10 to 4.88 (s, 2); 6.60 (d, 1); 7.27 (t, 2); 7.43 (m, 3); 7.56 to 7.70 (dd, 3); 7.82 (m, 4); 8.18 (d, 1); 8.20 to 8.50 (unresolved complex, 3); 11.00 (bs, 3).

### Example 91

### N-(2-Aminoethyl)-N-azetidin-3-yl-4-({4-[(4-fluorophenyl)amino]pyrimidin-2-yl}amino)benzene-sulfonamide hydrochloride

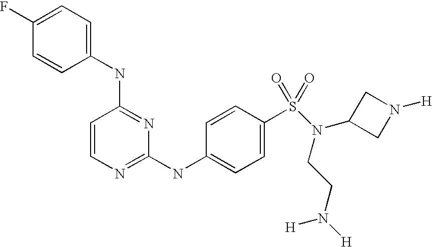

Stage 1: 3-(2-tert-Butoxycarbonylaminoethylamino)-azetidine-1-carboxylic acid tert-butyl ester: As in example A, starting with 1.7 g of 3-oxoazetidine-1-carboxylic acid tert-butyl ester and 1.6 g of (2-amino-ethyl)carbamic acid tert-butyl ester, 2 g of 3-(2-tert-butoxycarbonylaminoethylamino)azetidine-1-carboxylic acid tert-butyl ester are obtained.

Stage 2: 3-((2-tert-Butoxycarbonylaminoethyl)-{4-[4-(4-fluorophenylamino)pyrimidin-2-ylamino]benzenesulfonyl}-amino)azetidine-1-carboxylic acid tert-butyl ester: The procedure is carried out as in stage 4 of example 1, starting with 500 mg of 4-[4-(4-fluorophenylamino)-pyrimidin-2-ylamino]benzenesulfonyl chloride hydro-chloride and 420 mg of 3-(2-tert-butoxycarbonylamino-ethylamino)azetidine-1-carboxylic acid tert-butyl ester. 300 mg of expected product are thus obtained.

Stage 3: N-(2-Aminoethyl)-N-azetidin-3-yl-4-({4-[(4-fluorophenyl)amino]pyrimidin-2-yl}amino)benzene-sulfonamide hydrochloride: Starting with 300 mg of product obtained in stage 2, 255 mg of expected product are obtained after a decarboxylation reaction following procedure 2 of example 8.

MH+=458.1; Melting point>220° C. (Isopropyl ether)

1H NMR (DMSO): 3.07 (m, 2); 3.39 (m, 2); 3.92 to 4.19 (unresolved complex, 4); 4.54 (qui, 1); 6.55 (d, 1); 7.30 (t, 2); 7.67 (m, 2); 7.78 (d, 2); 7.92 (d, 2); 8.12 (d, 1); 8.20 (bs, 3); 9.27 (bs, 2); 10.87 (bs, 1); 11.04 (bs, 1).

### Example 92

### N-(3-Aminopropyl)-4-({4-[(4-fluorophenyl)-amino]pyrimidin-2-yl}amino)-N-piperidin-4-ylbenzene-sulfonamide hydrochloride

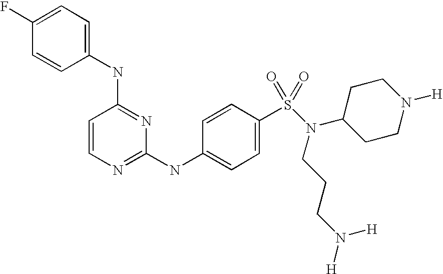

Stage 1: 4-(3-tert-Butoxycarbonylaminopropylamino)-piperidine-1-carboxylic acid tert-butyl ester: As in example A, starting with 3 g of 4-oxopiperidine-1-carboxylic acid tert-butyl ester and 2.62 g of (3-aminopropyl)carbamic acid tert-butyl ester, 4 g of 4-(3-tert-butoxycarbonylaminopropylamino)piperidine-1-carboxylic acid tert-butyl ester are obtained.

Stage 2: N-(3-Aminopropyl)-4-({4-[(4-fluorophenyl)-amino]pyrimidin-2-yl}amino)-N-piperidin-4-ylbenzene-sulfonamide hydrochloride: The procedure is carried out as in stage 4 of example 1, starting with 600 mg of 4-[4-(4-fluorophenylamino)pyrimidin-2-ylamino]benzenesulfonyl chloride hydrochloride and 600 mg of 4-(3-tert-butoxycarbonylaminopropylamino)piperidine-1-carboxylic acid tert-butyl ester. 163 mg of expected product are thus obtained after a decarboxylation reaction following procedure 2 of example 8.

MH+=500; Melting point=245-247° C. (Isopropyl ether)

1H NMR (DMSO): 1.53 (d, 2); 1.91 (qui, 2); 2.02 (qd, 2); 2.83 (sxt, 2); 2.95 (q, 2); 3.17 to 3.28 (unresolved complex, 4); 4.00 (m, 1); 6.52 (d, 1); 7.26 (t, 2); 7.66 (dd, 2); 7.78 to 7.85 (unresolved complex, 4); 7.99 (bs, 3); 8.10 (d, 1); 8.84 to 8.99 (unresolved complex, 2); 10.61 to 11.09 (unresolved complex, 2).

### Example 93

### N-[2-(Ethylamino)ethyl]-4-({4-[(4-fluoro-phenyl)amino]pyrimidin-2-yl}amino)-N-piperidin-4-yl-benzenesulfonamide hydrochloride

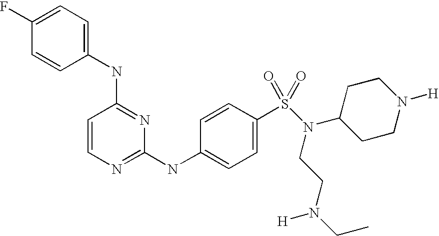

Stage 1: Ethyl-(2-hydroxyethyl)carbamic acid tert-butyl ester: A solution is prepared from 15.2 g of Boc2O and 30 ml of dichloromethane. This solution is added dropwise to a mixture containing 7.36 g of 2-ethylaminoethanol and 30 ml of dichloromethane in the cold state. The reaction medium is allowed to stand for 24 hours at room temperature. After concentrating to dryness, the reaction medium is taken up in an NaCl solution. The medium is extracted three times with ethyl acetate and then washed with a saturated NaCl solution. The organic phase is dried over sodium sulfate and then concentrated to dryness using a vane pump in order to give 12 g of expected product.

Stage 2: [2-(1,3-Dihydroisoindol-2-yl)ethyl]ethylcarbamic acid tert-butyl ester: A solution of 10.2 g of diethyl azodicarboxylate in 60 ml of THF is added dropwise to a mixture containing 11.92 g of ethyl-(2-hydroxyethyl)carbamic acid tert-butyl ester, 15.2 g of isoindole-1,3-dione and 8.6 g of triphenylphosphine in 40 ml of THF. The medium is kept stirring overnight at room temperature and then concentrated to dryness. The medium is taken up in 100 ml of ethyl ether, the precipitate formed is removed by filtration. The filtrate is concentrated and chromatographed on a silica column (heptane in a gradient up to 3% ethyl acetate). 14 g of expected product are obtained.

Stage 3: (2-Aminoethyl)ethylcarbamic acid tert-butyl ester: 3.2 g of hydrazine, 7.6 g of [2-(1,3-dihydro-isoindol-2-yl)ethyl]ethylcarbamic acid tert-butyl ester in 70 ml of ethanol are kept stirring at room temperature overnight. The precipitate formed is removed. The filtrate is concentrated to dryness, taken up in a potassium carbonate solution and extracted with dichloromethane. After drying on sodium sulfate and concentrating to dryness, 3.1 g of expected product are obtained.

Stage 4: 4-[2-(tert-Butoxycarbonylethylamino)ethyl-amino]piperidine-1-carboxylic acid tert-butyl ester: As in example A, starting with 3.3 g of 4-oxopiperidine-1-carboxylic acid tert-butyl ester and 3.1 g of (2-amino-ethyl)ethylcarbamic acid tert-butyl ester, 2.45 g of expected product are obtained.

Stage 5: N-[2-(Ethylamino)ethyl]-4-({4-[(4-fluoro-phenyl)amino]pyrimidin-2-yl}amino)-N-piperidin-4-yl-benzenesulfonamide hydrochloride: The procedure is carried out as in stage 4 of example 1, starting with 650 mg of 4-[4-(4-fluorophenylamino)pyrimidin-2-yl-amino]benzenesulfonyl chloride hydrochloride and 650 mg of 4-[2-(tert-butoxycarbonylethylamino)ethylamino]-piperidine-1-carboxylic acid tert-butyl ester. 254 mg of expected product are thus obtained after a decarboxylation reaction following procedure 2 of example 8.

MH+=514.1; Melting point=250° C. (Isopropyl ether)

1H NMR (DMSO): 1.23 (t, 3); 1.58 (d, 2); 1.93 (m, 2); 2.89 to 3.06 (unresolved complex, 4); 3.07 to 3.17 (unresolved complex, 2); 3.25 (d, 2); 3.42 (t, 3); 4.06 (m, 1); 6.55 (d, 1); 7.28 (t, 2); 7.66 (dd, 2); 7.79 to 7.90 (unresolved complex, 4); 8.11 (d, 1); 8.88 to 9.06 (unresolved complex, 2); 9.16 (bs, 2); 10.78 to 11.33 (bd, 2).

### Example 94

### N-(2-Aminoethyl)-4-({4-[(4-fluorophenyl)-amino]pyrimidin-2-yl}amino)-N-(tetrahydro-2H-pyran-4-yl)benzenesulfonamide hydrochloride

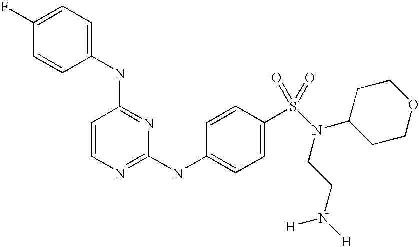

Stage 1: [2-(Tetrahydropyran-4-ylamino)ethyl]carbamic acid tert-butyl ester: As in example A, starting with 2.7 g of tetrahydropyran-4-one and 1.7 g of (2-amino-ethyl)carbamic acid tert-butyl ester, 2 g of expected product are obtained.

Stage 2: N-[2-(Ethylamino)ethyl]-4-({4-[(4-fluoro-phenyl)amino]pyrimidin-2-yl}amino)-N-piperidin-4-yl-benzenesulfonamide hydrochloride: The procedure is carried out as in stage 4 of example 1, starting with 460 mg of 4-[4-(4-fluorophenylamino)pyrimidin-2-yl-amino]benzenesulfonyl chloride hydrochloride and 300 mg of [2-(tetrahydropyran-4-ylamino)ethyl]carbamic acid tert-butyl ester. 254 mg of expected product are thus obtained after a decarboxylation reaction following procedure 2 of example 8.

MH+=487.0; Melting point=200° C. (Isopropyl ether)

1H NMR (DMSO): 1.32 (d, 2); 1.59 (qd, 2); 2.96 (m, 2); 3.25 to 3.39 (unresolved complex, 4); 3.82 (dd, 2); 3.93 (m, 1); 6.47 (d, 1); 7.25 (t, 2); 7.66 (dd, 2); 7.80 (d, 2); 7.86 (d, 2); 7.94 (bs, 3); 8.10 (d, 1); 10.55 (bs, 1); 10.65 (bs, 1).

### Example 95

### N-(2-Aminoethyl)-4-({4-[(4-fluoro-3-methyl-phenyl)amino]pyrimidin-2-yl}amino)-N-(1-methylpiperidin-4-yl)benzenesulfonamide hydrochloride

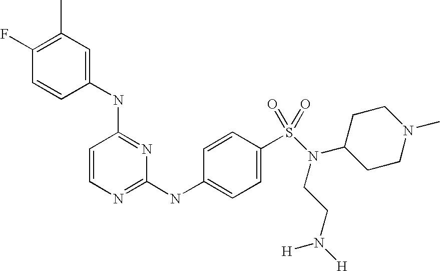

Stage 1: [2-(1-Methylpiperidin-4-ylamino)ethyl]carbamic acid tert-butyl ester: As in example A, starting with 2.7 g of 1-methylpiperidin-4-one and 1.7 g of (2-amino-ethyl)carbamic acid tert-butyl ester, 2.2 g of expected product are obtained.

Stage 2: N-(2-Aminoethyl)-4-({4-[(4-fluoro-3-methyl-phenyl)amino]pyrimidin-2-yl}amino)-N-(1-methylpiperidin-4-yl)benzenesulfonamide hydrochloride: The procedure is carried out as in stage 4 of example 1, starting with 380 mg of 4-({4-[(4-fluoro-3-methylphenyl)amino]-pyrimidin-2-yl}amino)benzenesulfonyl chloride hydro-chloride and 300 mg of [2-(1-methylpiperidin-4-ylamino)ethyl]carbamic acid tert-butyl ester. 168 mg of expected product are thus obtained after a decarboxylation reaction following procedure 2 of example 8.

MH+=514.2; Melting point=220° C. (Isopropyl ether)

1H NMR (DMSO): 1.44 (d, 0.3); 1.63 (d, 1.7); 1.99 (q, 2); 2.23 (d, 3); 2.66 (d, 3); 2.92 to 3.18 (unresolved complex, 4); 3.23 to 3.42 (unresolved complex, 4); 4.00 (m, 1); 6.52 (d, 1); 7.20 (t, 1); 7.44 (m, 1); 7.60 (dd, 1); 7.81 (d, 2); 7.88 (d, 2); 8.11 (d, 1); 8.07 to 8.28 (unresolved complex, 3); 10.74 (bs, 2); 10.95 (bs, 1).

### Example 96

### N-Azetidin-3-yl-4-({4-[(4-fluorophenyl)-amino]pyrimidin-2-yl}amino)-N-(2-pyrrolidin-1-ylethyl)-benzenesulfonamide hydrochloride

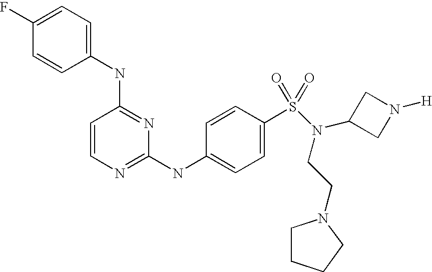

Stage 1: 3-(2-Pyrrolidin-1-ylethylamino)azetidine-1-carboxylic acid tert-butyl ester: As in example A, starting with 3.32 g of 3-oxoazetidine-1-carboxylic acid tert-butyl ester and 2.5 ml of 2-pyrrolidin-1-yl-ethylamine, 1.15 g of expected product are obtained.

Stage 2: N-[2-(Ethylamino)ethyl]-4-({4-[(4-fluoro-phenyl)amino]pyrimidin-2-yl}amino)-N-piperidin-4-yl-benzenesulfonamide hydrochloride: The procedure is carried out as in stage 4 of example 1, starting with 780 mg of 4-[4-(4-fluorophenylamino)pyrimidin-2-ylamino]benzenesulfonyl chloride hydrochloride and 560 mg of 3-(2-pyrrolidin-1-ylethylamino)azetidine-1-carboxylic acid tert-butyl ester. 230 mg of expected product are thus obtained after a decarboxylation reaction following procedure 2 of example 8.

MH+=512.1; Melting point=215° C. (Isopropyl ether)

1H NMR (DMSO): 1.78 to 2.12 (unresolved complex, 4); 3.10 (m, 2); 3.39 to 3.67 (unresolved complex, 6); 3.98 to 4.16 (unresolved complex, 4); 4.64 (qui, 1); 6.54 (d, 1); 7.32 (t, 2); 7.68 (dd, 2); 7.84 (d, 2); 7.91 (d, 2); 8.12 (d, 1); 9.22 (bs, 1); 9.39 (bs, 1); 10.84 (bs, 1); 11.00 (bs, 1); 11.17 (bs, 1).

### Example 97

### N-Azetidin-3-yl-4-({4-[(4-fluoro-3-methyl-phenyl)amino]pyrimidin-2-yl}amino)-N-(2-pyrrolidin-1-ylethyl)benzenesulfonamide hydrochloride

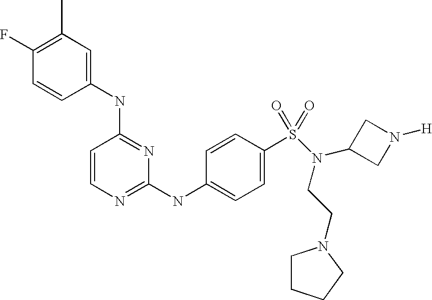

Stage 1: 3-(2-Pyrrolidin-1-ylethylamino)azetidine-1-carboxylic acid tert-butyl ester: As in example A, starting with 3.32 g of 3-oxoazetidine-1-carboxylic acid tert-butyl ester and 2.5 ml of 2-pyrrolidin-1-yl-ethylamine, 1.15 g of expected product are obtained.

Stage 2: N-Azetidin-3-yl-4-({4-[(4-fluoro-3-methyl-phenyl)amino]pyrimidin-2-yl}amino)-N-(2-pyrrolidin-1-ylethyl)benzenesulfonamide hydrochloride: The procedure is carried out as in stage 4 of example 1, starting with 660 mg of 4-({4-[(4-fluoro-3-methylphenyl)amino]-pyrimidin-2-yl}amino)benzenesulfonyl chloride hydro-chloride and 440 mg of 3-(2-pyrrolidin-1-ylethyl-amino)azetidine-1-carboxylic acid tert-butyl ester. 264 mg of expected product are thus obtained after a decarboxylation reaction following procedure 2 of example 8.

MH+=526.2; Melting point=231-235° C. (Isopropyl ether)

1H NMR (DMSO): 1.76 to 2.11 (unresolved complex, 4); 2.23 (d, 3); 3.10 (m, 2); 3.38 to 3.68 (unresolved complex, 6); 4.06 (q, 4); 4.64 (qui, 1); 6.55 (d, 1); 7.25 (t, 1); 7.46 (m, 1); 7.57 (dd, 1); 7.83 (d, 2); 7.91 (d, 2); 8.11 (d, 1); 9.23 (bs, 1); 9.40 (bs, 1); 10.85 (bs, 1); 10.99 to 11.27 (unresolved complex, 2).

### Example 98

### N-(2-Aminoethyl)-N-azetidin-3-yl-4-({4-[(4-fluoro-3-methylphenyl)amino]pyrimidin-2-yl}amino)-benzenesulfonamide hydrochloride

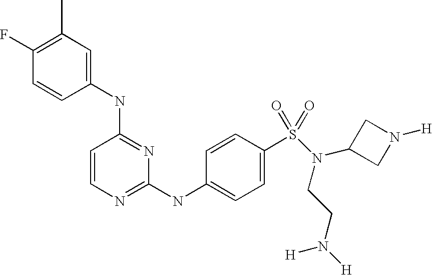

Stage 1: 3-(2-tert-Butoxycarbonylaminoethylamino)-azetidine-1-carboxylic acid tert-butyl ester: As in example A, starting with 1.7 g of 3-oxoazetidine-1-carboxylic acid tert-butyl ester and 1.6 g of (2-amino-ethyl)carbamic acid tert-butyl ester, 2 g of expected product are obtained.

Stage 2: N-(2-Aminoethyl)-N-azetidin-3-yl-4-({4-[(4-fluoro-3-methylphenyl)amino]pyrimidin-2-yl}amino)-benzenesulfonamide hydrochloride: The procedure is carried out as in stage 4 of example 1, starting with 800 mg of 4-({4-[(4-fluoro-3-methylphenyl)amino]-pyrimidin-2-yl}amino)benzenesulfonyl chloride hydro-chloride and 635 mg of 3-(2-tert-butoxycarbonylamino-ethylamino)azetidine-1-carboxylic acid tert-butyl ester. 350 mg of expected product are thus obtained after a decarboxylation reaction following procedure 2 of example 8.

MH+=472.1; Melting point=205° C. (Isopropyl ether)

1H NMR (DMSO): 2.23 (d, 3); 3.07 (m, 2); 3.34 (t, 2); 3.95 to 4.19 (unresolved complex, 4); 4.53 (qui, 1); 6.51 (d, 1); 7.21 (t, 1); 7.46 (m, 1); 7.58 (dd, 1); 7.76 (d, 2); 7.94 (d, 2); 8.11 (d, 1); 8.09 to 8.23 (bs, 3); 9.19 (bs, 2); 10.59 (bs, 1); 10.86 (bs, 1).

### Example 99

### N-(2-Aminoethyl)-4-({4-[(4-fluoro-3-methyl-phenyl)amino]pyrimidin-2-yl}amino)-N-[1-(2,2,2-tri-fluoroethyl)piperidin-4-yl]benzenesulfonamide hydrochloride

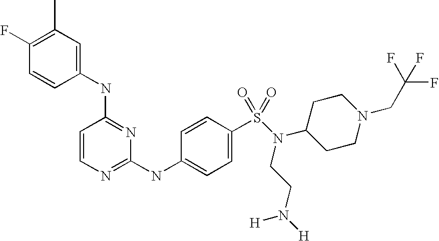

Stage 1:1-(2,2,2-Trifluoroethyl)piperidin-4-one: A mixture containing 1.6 g of piperidin-4-one hydrochloride and 2.6 g of sodium hydrogen carbonate in 15 ml of ethanol is kept stirring for 10 minutes. Nitrogen is bubbled through for 2 minutes and then 2.3 g of trifluoromethanesulfonic acid 2,2,2-trifluoroethyl ester are added. The reaction medium is kept stirring at 80° C. for 6 hours. After concentrating to a third, a potassium carbonate solution is added and then the medium is extracted three times with dichloromethane. The organic phase is dried over sodium sulfate, concentrated under vacuum and chromatographed (1% methanol in dichloromethane) on a silica column. 1.3 g of expected product are obtained.

Stage 2: {2-[1-(2,2,2-Trifluoroethyl)piperidin-4-yl-amino]ethyl}carbamic acid tert-butyl ester: As in example A, starting with 1.3 g of 1-(2,2,2-trifluoro-ethyl)piperidin-4-one and 1.3 g of (2-aminoethyl)carbamic acid tert-butyl ester, 2.2 g of expected product are obtained.

Stage 3: N-(2-Aminoethyl)-4-({4-[(4-fluoro-3-methyl-phenyl)amino]pyrimidin-2-yl}amino)-N-[1-(2,2,2-tri-fluoroethyl)piperidin-4-yl]benzenesulfonamide hydro-chloride: The procedure is carried out as in stage 4 of example 1, starting with 700 mg of 4-({4-[(4-fluoro-3-methylphenyl)amino]pyrimidin-2-yl}amino)benzenesulfonyl chloride hydrochloride and 500 mg of {2-[1-(2,2,2-trifluoroethyl)piperidin-4-ylamino]ethyl}carbamic acid tert-butyl ester. 135 mg of expected product are thus obtained after a decarboxylation reaction following procedure 2 of example 8.

MH+=582.1; Melting point=225° C. (Isopropyl ether)

1H NMR (DMSO): 1.40 (d, 2); 1.76 (m, 2); 2.22 (s, 3); 2.59 (m, 2); 2.88 to 3.09 (unresolved complex, 4); 3.28 to 3.52 (unresolved complex, 4); 3.78 (m, 1); 6.61 (d, 1); 7.22 (t, 1); 7.41 (m, 1); 7.58 (bd, 1); 7.73 to 7.88 (unresolved complex, 4); 7.98 to 8.15 (unresolved complex, 4); 11.25 (bs, 1); 11.38 (bs, 1).

### Example 100

### N-(2-Aminoethyl)-4-({4-[(4-fluorophenyl)-amino]pyrimidin-2-yl}amino)-N-[1-(2,2,2-trifluoro-ethyl)piperidin-4-yl]benzenesulfonamide hydrochloride

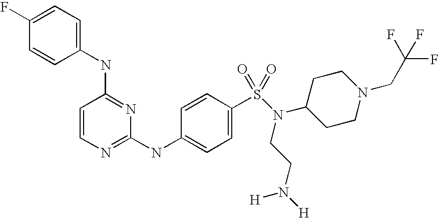

The procedure is carried out as in stage 4 of example 1, starting with 600 mg of 4-({4-[(4-fluorophenyl)-amino]pyrimidin-2-yl}amino)benzenesulfonyl chloride hydrochloride and 515 mg of {2-[1-(2,2,2-trifluoro-ethyl)piperidin-4-ylamino]ethyl}carbamic acid tert-butyl ester. 193 mg of expected product are thus obtained after a decarboxylation reaction following procedure 2 of example 8.

MH+=568; Melting point=240-245° C. (Isopropyl ether)

1H NMR (DMSO): 1.40 (d, 2); 1.75 (q, 2); 2.48 to 2.71 (unresolved complex, 2); 2.87 to 3.09 (unresolved complex, 4); 3.20 to 3.55 (unresolved complex, 4); 3.77 (m, 1); 6.60 (d, 1); 7.29 (t, 2); 7.64 (m, 2); 7.78 (d, 2); 7.85 (d, 2); 8.07 (bs, 2); 8.12 (d, 2); 11.27 (bs, 1); 11.35 (bs, 1).

### Example 101

### N-(1-Benzylpiperidin-4-yl)-4-({4-[(4-fluorophenyl)amino]pyrimidin-2-yl}amino)-N-{2-[(2-hydroxy-2-methylpropyl)amino]ethyl}benzenesulfonamide hydrochloride

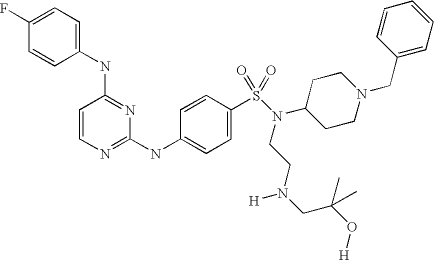

220 mg of the product of example 90, regenerated as a base, are reacted in the presence of 0.05 ml of 2,2-dimethyloxirane in a microwave (150° C., 200 W) for 15 minutes. The reaction medium is taken up in a potassium carbonate solution and extracted with dichloromethane. After drying over sodium sulfate but concentrating under vacuum, the crude product is chromatographed on a silica column (dichloromethane in a gradient up to 8% methanol). A product is thus obtained which is triturated in 2N hydrochloric ether in order to give 128 mg of expected hydrochloride.

MH+=648.2; Melting point: 200° C. (Ethyl ether)

1H NMR (DMSO): 1.22 (s, 5); 1.27 (s, 1); 1.44 (d, 0.4); 1.62 (d, 1.6); 2.21 (q, 2); 2.92 to 3.10 (unresolved complex, 4); 3.16 (m, 2); 3.29 (d, 2); 3.49 (t, 2); 4.05 (m, 1); 4.22 (d, 2); 6.51 (d, 1); 7.26 (t, 2); 7.39 to 7.48 (unresolved complex, 3); 7.61 (dd, 2); 7.67 (dd, 2); 7.75 to 7.90 (unresolved complex, 4); 8.11 (d, 1); 8.74 (bs, 1.6); 9.15 (bs, 0.4); 10.58 to 11.01 (unresolved complex, 2); 11.20 (bs, 1).

### Example 102

### 4-({4-[(4-Fluorophenyl)amino]pyrimidin-2-yl}amino)-N-{2-[(2-hydroxy-2-methylpropyl)amino]ethyl}-N-piperidin-4-ylbenzenesulfonamide

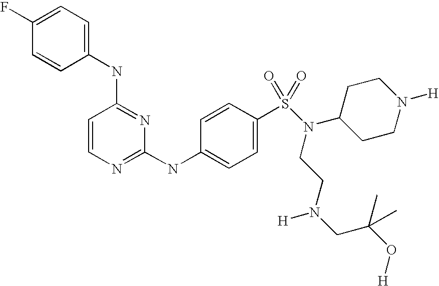

A mixture containing 230 mg of N-(1-benzylpiperidin-4-yl)-4-({4-[(4-fluorophenyl)amino]pyrimidin-2-yl}amino)-N-{2-[(2-hydroxy-2-methylpropyl)amino]ethyl}benzene-sulfonamide hydrochloride (example 101), 50 mg of palladium on carbon (10%) in 20 ml ethanol is left at 35° C. under a hydrogen atmosphere for 24 hours. After filtration, concentration of the filtrate and trituration in isopropyl ether, 132 mg of expected product are obtained.

MH+=558.2; Melting point: 120° C. (Isopropyl ether)

1H NMR (DMSO): 1.09 (s, 6); 1.36 (d, 2); 1.46 to 1.59 (m, 2); 2.43 (bs, 2); 2.46 to 2.55 (unresolved complex, 2); 2.73 (t, 2); 2.96 (d, 2); 3.15 (t, 2); 3.66 (m, 1); 4.14 (bs, 1); 6.30 (d, 1); 7.17 (t, 2); 7.63 to 7.74 (unresolved complex, 4); 7.94 (d, 2); 8.08 (d, 1); 9.49 (bs, 1); 9.64 (bs, 1).

### Example 103

### N-(1-Benzylpiperidin-4-yl)-4-({4-[(4-fluorophenyl)amino]pyrimidin-2-yl}amino)-N-{2-[(2,2,2-trifluoroethyl)amino]ethyl}benzenesulfonamide

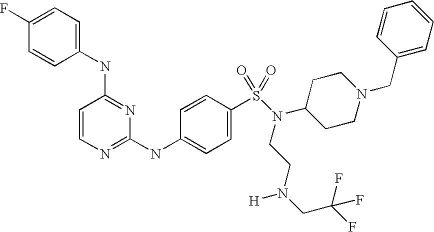

Following the process described in stage 1 of example 99, the reaction uses 420 mg of N-(2-aminoethyl)-N-(1-benzylpiperidin-4-yl)-4-({4-[(4-fluorophenyl)amino]pyrimidin-2-yl}amino)benzene-sulfonamide hydrochloride (example 90) and 170 mg of trifluoromethanesulfonic acid 2,2,2-trifluoroethyl ester. 140 mg of expected product are obtained after recrystallization.

MH+=658.1; Melting point: 137-138° C. (Isopropyl ether/dichloromethane)

1H NMR (DMSO): 1.31 (d, 2); 1.59 (q, 2); 1.91 (t, 2); 2.47 (m, 1); 2.69 to 2.86 (unresolved complex, 4); 3.12 (t, 2); 3.26 (dd, 2); 3.40 (bs, 2); 3.52 (m, 1); 6.29 (d, 1); 7.11 to 7.36 (unresolved complex, 7); 7.62 to 7.76 (unresolved complex, 4); 7.94 (d, 2); 8.08 (d, 1); 9.49 (bs, 1); 9.66 (bs, 1).

### Example 104

### N-(1-Ethylazetidin-3-yl)-4-({4-[(4-fluoro-3-methylphenyl)amino]pyrimidin-2-yl}amino)-N-(2-pyrrolidin-1-ylethyl)benzenesulfonamide

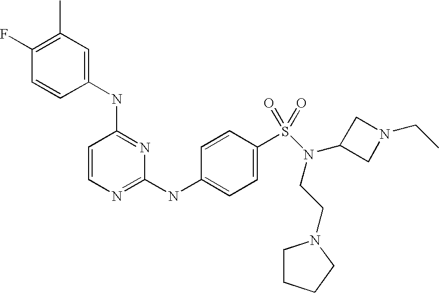

As in example A, starting with 160 mg of regenerated N-azetidin-3-yl-4-({4-[(4-fluoro-3-methylphenyl)amino]-pyrimidin-2-yl}amino)-N-(2-pyrrolidin-1-ylethyl)benzene-sulfonamide (example 96) and 0.02 ml of acetaldehyde, 75 mg of expected product are obtained.

MH+=554.2; Melting point=195° C. (Isopropyl ether)

1H NMR (DMSO): 1.08 (t, 3); 1.81 to 2.10 (unresolved complex, 4); 2.24 (s, 3); 2.92 to 3.31 (unresolved complex, 5); 3.33 to 3.99 (unresolved complex, 5); 4.08 to 4.28 (unresolved complex, 4); 4.45 (m, 0.7); 4.90 (m, 0.3); 6.55 (d, 1); 7.24 (t, 1); 7.46 (bs, 1); 7.56 (d, 1); 7.83 (d, 2); 7.90 (d, 2); 8.11 (d, 1); 10.86 (bs, 1); 11.07 (bs, 2); 11.25 (bs, 0.4); 11.50 (bs, 0.6).

### Example 105

### 4-({4-[(3,4-Difluorophenyl)amino]pyrimidin-2-yl}amino)-N-methyl-N-piperidin-4-ylbenzenesulfonamide hydrochloride

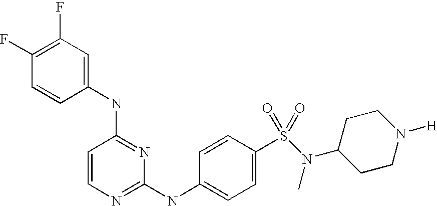

The procedure is carried out as in stage 4 of example 1, starting with 1.95 g of 4-({4-[(3,4-di-fluorophenyl)amino]pyrimidin-2-yl}amino)benzenesulfonyl chloride hydrochloride and 1.1 g of 4-methylamino-piperidine-1-carboxylic acid tert-butyl ester. 1.25 g of expected product are thus obtained after a decarboxylation reaction following procedure 2 of example 8.

MH+=475.0; Melting point=275° C. (Isopropyl ether)

1H NMR (DMSO): 1.45 (d, 2); 1.84 (qd, 2); 2.68 (s, 3); 2.97 (q, 2); 3.24 (d, 2); 4.08 (m, 1); 6.51 (d, 1); 7.33 (m, 1); 7.44 (q, 1); 7.77 (d, 2); 7.86 (d, 2); 8.01 (dd, 1); 8.13 (d, 1); 8.54 (ql, 1); 8.77 (bd, 1); 10.64 (bs, 2).

### Example 106

### 4-({4-[(4-Fluoro-3-methylphenyl)amino]-pyrimidin-2-yl}amino)-N-methyl-N-piperidin-4-ylbenzene-sulfonamide hydrochloride

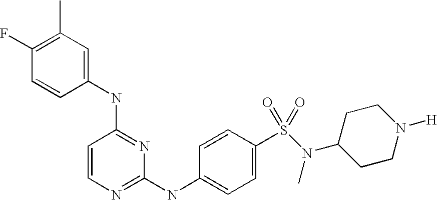

The procedure is carried out as in stage 4 of example 1, starting with 3 g of 4-({4-[(4-fluoro-3-methylphenyl)amino]pyrimidin-2-yl}amino)benzenesulfonyl chloride hydrochloride and 1.8 g of 4-methylamino-piperidine-1-carboxylic acid tert-butyl ester. 1.86 g of expected product are thus obtained after a decarboxylation reaction following procedure 2 of example 8.

MH+=471.1; Melting point=215-220° C. (Isopropyl ether)

1H NMR (DMSO): 1.42 (d, 2); 1.87 (qd, 2); 2.22 (s, 3); 2.67 (s, 3); 2.95 (q, 2); 3.23 (d, 2); 4.09 (m, 1); 6.58 (d, 1); 7.21 (t, 1); 7.42 (m, 1); 7.59 (dd, 2); 7.73 to 7.86 (unresolved complex, 4); 8.11 (d, 1); 8.79 (bd, 1); 8.93 (bd, 1); 11.09 (bs, 1); 11.23 (bs, 1).

### Example 107

### 4-({4-[(4-Fluorophenyl)amino]pyrimidin-2-yl}amino)-N-methyl-N-[1-(3,3,3-trifluoropropyl)piperidin-4-yl]benzenesulfonamide

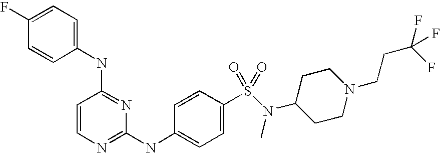

As in example A, starting with 460 mg of regenerated 4-({4-[(4-fluorophenyl)amino]pyrimidin-2-yl}amino)-N-methyl-N-piperidin-4-ylbenzenesulfonamide (example 8) and 160 mg of 3,3,3-trifluoropropionaldehyde, 113 mg of expected product are obtained.

MH+=553; Melting point=195-196° C. (Isopropyl ether/dichloromethane)

1H NMR (DMSO): 1.22 (d, 2); 1.54 (m, 2); 1.96 (t, 2); 2.30 to 2.48 (unresolved complex, 4); 2.65 (s, 3); 2.84 (d, 2); 3.63 (m, 1); 6.29 (d, 1); 7.18 (t, 2); 7.64 (d, 2); 7.71 (dd, 2); 7.96 (d, 2); 8.09 (d, 1); 9.49 (bs, 1); 9.68 (bs, 1).

### Example 108

### 4-({4-[(3,4-Difluorophenyl)amino]pyrimidin-2-yl}amino)-N-methyl-N-[1-(4,4,4-trifluorobutyl)-piperidin-4-yl]benzenesulfonamide

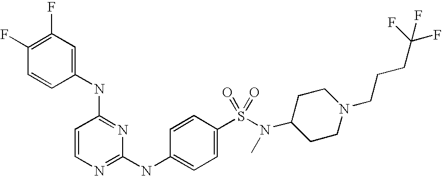

As in example A, starting with 360 mg of regenerated 4-({4-[(3,4-difluorophenyl)amino]pyrimidin-2-yl}amino)-N-methyl-N-piperidin-4-ylbenzenesulfonamide (example 105) and 90 mg of 4,4,4-trifluorobutyraldehyde, 205 mg of expected product are obtained.

MH+=585; Melting point=192° C. (Isopropyl ether/dichloromethane)

1H NMR (DMSO): 1.24 (d, 2); 1.50 to 1.64 (unresolved complex, 4); 1.90 (t, 2); 2.15 to 2.25 (unresolved complex, 2); 2.28 (t, 2); 2.67 (s, 3); 2.79 (d, 2); 3.62 (m, 1); 6.32 (d, 1); 7.29 (m, 1); 7.38 (q, 1); 7.66 (d, 2); 7.94 (d, 2); 8.01 (m, 1); 8.12 (d, 1); 9.66 (bs, 1); 9.75 (bs, 1).

### Example 109

### 4-({4-[(3,4-Difluorophenyl)amino]pyrimidin-2-yl}amino)-N-methyl-N-[1-(3,3,3-trifluoro-propyl)piperidin-4-yl]benzenesulfonamide

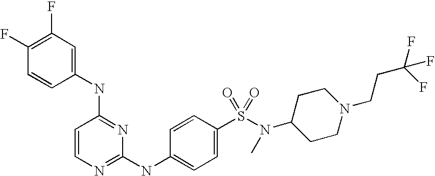

As in example A, starting with 360 mg of regenerated 4-({4-[(3,4-difluorophenyl)amino]pyrimidin-2-yl}amino)-N-methyl-N-piperidin-4-ylbenzenesulfonamide (example 105) and 80 mg of 3,3,3-trifluoropropionaldehyde, 100 mg of expected product are obtained.

MH+=571; Melting point=202-203° C. (Isopropyl ether/dichloromethane)

1H NMR (DMSO): 1H NMR (DMSO): 1.23 (d, 2); 1.55 (qd, 2); 1.95 (t, 2); 2.26-2.53 (unresolved complex, 4); 2.66 (s, 3); 2.83 (d, 2); 3.62 (m, 1); 6.33 (d, 1); 7.24-7.46 (unresolved complex, 2); 7.67 (d, 2); 7.96 (d, 2); 8.06-8.20 (unresolved complex, 2); 9.70 (s, 1); 9.78 (s, 1).

### Example 110

### 4-({4-[(4-Fluoro-3-methylphenyl)amino]-pyrimidin-2-yl}amino)-N-methyl-N-{1-[(1-methyl-1H-pyrrol-2-yl)methyl]piperidin-4-yl}benzenesulfonamide

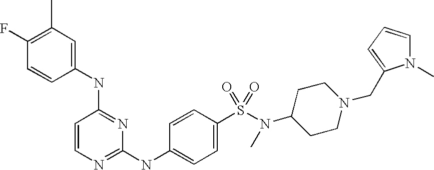

As in example A, starting with 400 mg of regenerated 4-({4-[(4-fluoro-3-methylphenyl)amino]pyrimidin-2-yl}-amino)-N-methyl-N-piperidin-4-ylbenzenesulfonamide (example 106) and 90 mg of 1-methyl-1H-pyrrole-2-carbaldehyde, 208 mg of expected product are obtained.

MH+=564.1; Melting point=159-160° C. (Isopropyl ether/dichloromethane)

1H NMR (DMSO): 1.23 (d, 2); 1.52 (qd, 2); 1.87 (t, 2); 2.25 (s, 3); 2.64 (s, 3); 2.78 (d, 2); 3.31 (s, 2); 3.53 (s, 3); 3.61 (m, 1); 5.79-5.86 (unresolved complex, 2); 6.28 (d, 1); 6.62 (t, 1); 7.11 (t, 1); 7.46 (m, 1); 7.58 (dd, 1); 7.62 (d, 2); 7.95 (d, 2); 8.08 (d, 1); 9.41 (s, 1); 9.67 (s, 1).

### Example 111

### 4-({4-[(4-Fluoro-3-methylphenyl)amino]-pyrimidin-2-yl}amino)-N-methyl-N-{1-[(1H-pyrrol-3-yl)-methyl]piperidin-4-yl}benzenesulfonamide

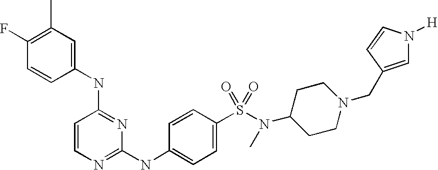

As in example A, starting with 500 mg of regenerated 4-({4-[(4-fluoro-3-methylphenyl)amino]pyrimidin-2-yl}-amino)-N-methyl-N-piperidin-4-ylbenzenesulfonamide (example 106) and 120 mg of 1H-pyrrole-3-carbaldehyde, 144 mg of expected product are obtained.

MH+=550.1; Melting point=134-139° C. (Isopropyl ether/dichloromethane)

1H NMR (DMSO): 1.18 (d, 2); 1.52 (m, 2); 1.80 (t, 2); 2.23 (d. 3); 2.63 (s, 3); 2.79 (d, 2); 3.22 (s, 2); 3.54 (m, 1), 5.88 (m, 1); 6.27 (d, 1); 6.56 (m, 1); 6.62 (m, 1); 7.10 (t, 1); 7.45 (m, 1); 7.52-7.67 (unresolved complex, 3), 7.93 (d, 2); 8.06 (d, 1); 9.41 (s, 1); 9.67 (s, 1); 10.54 (bs, 1).

### Example 112

### 4-({4-[(4-Fluorophenyl)amino]pyrimidin-2-yl}amino)-N-methyl-N-[1-(2,2,2-trifluoroethyl)piperidin-4-yl]benzenesulfonamide

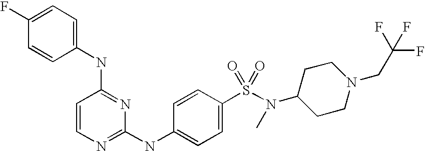

Following the procedure described in stage 1 of example 99, the reaction uses 456 mg of 4-({4-[(4-fluorophenyl)amino]pyrimidin-2-yl}amino)-N-methyl-N-piperidin-4-ylbenzenesulfonamide hydrochloride and 240 mg of trifluoromethanesulfonic acid 2,2,2-trifluoroethyl ester. 404 mg of expected product are obtained after recrystallization.

MH+=539; Melting point: 201-202° C. (Isopropyl ether/dichloromethane)

1H NMR (DMSO): 1.21 (d, 2); 1.59 (qd, 2); 2.34 (t, 2); 2.66 (s, 3); 2.87 (d, 2); 3.11 (q, 2); 3.65 (m, 1); 6.29 (d, 1); 7.18 (t, 2); 7.65 (d, 2); 7.70 (dd, 2); 7.96 (d, 2); 8.09 (d, 1); 9.49 (bs, 1); 9.67 (bs, 1).

### Example 113

### 4-({4-[(4-Fluorophenyl)amino]pyrimidin-2-yl}amino)-N-methyl-N-(tetrahydro-2H-thiopyran-4-yl)-benzenesulfonamide

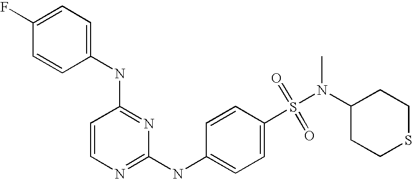

Stage 1: Methyl-(tetrahydrothiopyran-4-yl)amine: As in example A, starting with 5 g of tetrahydrothiopyran-4-one, 43 ml of methylamine as a 2M solution in THF, 600 mg of expected product are obtained.

Stage 2: The procedure is carried out as in stage 4 of example 1, starting with 600 mg of 4-({4-[(4-fluoro-phenyl)amino]pyrimidin-2-yl}amino)benzenesulfonyl chloride hydrochloride and 600 mg of methyl-(tetrahydrothiopyran-4-yl)amine. 340 mg of expected product are thus obtained.

MH+=474.1; Melting point: 181.5° C. (Isopropyl ether)

1H NMR (DMSO): 1.70 (m, 4); 2.60 to 2.88 (unresolved complex, 4); 2.74 (s, 3); 3.77 (m, 1); 6.37 (d, 1); 7.26 (t, 2); 7.74 (d, 2); 7.79 (m, 2); 8.04 (d, 2); 8.17 (d, 1); 9.58 (bs, 1); 9.77 (bs, 1).

### Example 114

### N-[2-(Dimethylamino)ethyl]-N-(1,1-dioxido-tetrahydro-3-thienyl)-4-({4-[(4-fluorophenyl)amino]-pyrimidin-2-yl}amino)benzenesulfonamide

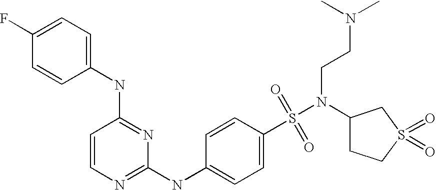

The procedure is carried out as in stage 4 of example 1 starting with 600 mg of 4-({4-[(4-fluorophenyl)amino]-pyrimidin-2-yl}amino)benzenesulfonyl chloride hydro-chloride which are reacted with 450 mg of [2-(dimethyl-amino)ethyl]-N-(1,1-dioxidotetrahydro-3-thienyl). 90 mg of expected product are thus obtained.

MH+=549.0; Melting point: 162-165° C. (Isopropyl ether)

1H NMR (DMSO): 2.09 (m, 2); 2.31 (bs, 6); 2.53-3.38 (unresolved complex, 8); 4.66 (q, 1); 6.30 (d, 1); 7.18 (t, 2); 7.62-7.79 (unresolved complex, 4); 8.00 (d, 2); 8.10 (d, 1); 9.53 (s, 1); 9.75 (s, 1).

### Example 115

### N-1,4-Dioxaspiro[4.5]dec-8-yl-4-({4-[(4-fluorophenyl)amino]pyrimidin-2-yl}amino)-N-methyl-benzenesulfonamide

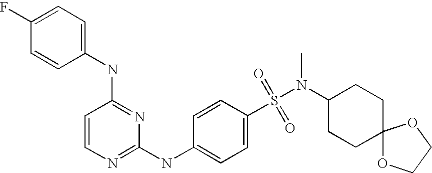

Stage 1: Benzyl-(1,4-dioxaspiro[4.5]dec-8-yl)methylamine: As in example A, starting with 10 g of 1,4-dioxaspiro[4.5]decan-8-one and 8.5 g of N-benzylmethyl-amine as a 2M solution in THF, 10 g of expected product are obtained.

Stage 2: N-Methyl-1,4-dioxaspiro[4.5]decan-8-amine: To a solution of 10 g of benzyl-(1,4-dioxaspiro[4.5]dec-8-yl)methylamine in 280 ml of ethanol containing 6.6 ml of acetic acid are added under an argon atmosphere 10 g of palladium on carbon and 18 ml of 1,4-cyclohexanediene. The medium is kept stirring for 18 hours at room temperature. After filtration on celite and concentration under vacuum, the medium is taken up in a saturated sodium carbonate solution and extracted with ethyl acetate. The organic phase is dried over sodium sulfate. 4.7 g of product are obtained after concentrating to dryness.

Stage 3: N-1,4-Dioxaspiro[4.5]dec-8-yl-4-({4-[(4-fluorophenyl)amino]pyrimidin-2-yl}amino)-N-methyl-benzenesulfonamide: The procedure is carried out as in stage 4 of example 1 starting with 4-({4-[(4-fluoro-phenyl)amino]pyrimidin-2-yl}amino)benzenesulfonyl chloride hydrochloride which is reacted with 4.5 g of N-methyl-1,4-dioxaspiro[4.5]decan-8-amine. 7 g of expected product are thus obtained.

1H NMR (DMSO): 1.20 (m, 2); 1.52 (m, 6); 2.57 (s, 3); 3.71 (m, 1); 3.75 (s, 4); 6.24 (d, 1); 7.10 (t, 2); 7.61 (d, 2); 7.64 (d, 1); 7.67 (d, 1); 7.89 (d, 2); 8.04 (d, 1); 9.49 (bs, 1); 9.66 (bs, 1).

### Example 116

### N-1,4-Dioxaspiro[4.5]dec-8-yl-4-({4-[(4-fluorophenyl)amino]pyrimidin-2-yl}amino)-N-(2-pyrrolidin-1-ylethyl)benzenesulfonamide

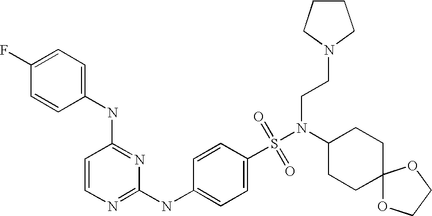

Stage 1: (2-Pyrrolidin-1-ylethyl)(1,4-dioxaspiro[4.5]dec-8-yl)amine: As in example A, starting with 10 g of 1,4-dioxaspiro[4.5]decan-8-one and 8 g of 2-pyrrolidin-1-ylethylamine, 12 g of expected product are obtained.

Stage 2: N-1,4-dioxaspiro[4.5]dec-8-yl-4-({4-[(4-fluorophenyl)amino]pyrimidin-2-yl}amino)-N-(2-pyrrolidin-1-ylethyl)benzenesulfonamide: The procedure is carried out as in stage 4 of example 1 starting with 2 g of 4-({4-[(4-fluorophenyl)amino]pyrimidin-2-yl}-amino)benzenesulfonyl chloride hydrochloride and 740 mg of (2-pyrrolidin-1-yl-ethyl)(1,4-dioxaspiro[4.5]dec-8-yl)amine. 1.3 g of expected product are thus obtained.

MH+=572.2; Melting point: 155° C. (Isopropyl ether)

1H NMR (DMSO): 1.29-1.77 (unresolved complex, 12); 2.45 (m, 4); 2.57 (t, 2); 3.13 (t, 2); 3.67 (m, 1); 3.81 (s, 4); 6.29 (d, 1); 7.17 (t, 2); 7.60-7.77 (unresolved complex, 4); 7.93 (d, 2); 8.08 (d, 1); 9.48 (s, 1); 9.66 (s, 1).

### Example 117

### 4-({4-[(4-Fluorophenyl)amino]pyrimidin-2-yl}amino)-N-(4-oxocyclohexyl)-N-(2-pyrrolidin-1-yl-ethyl)benzenesulfonamide

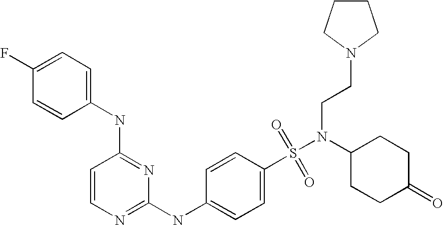

To a solution containing 1 g of N-1,4-dioxaspiro[4.5]dec-8-yl-4-({4-[(4-fluorophenyl)amino]pyrimidin-2-yl}amino)-N-(2-pyrrolidin-1-ylethyl)benzenesulfonamide (example 116) in 10 ml of THF are added 15 ml of a 3N hydrochloric acid solution. The reaction medium is left for 18 hours at room temperature. The medium is neutralized with a dilute sodium hydroxide solution, extracted with ethyl acetate and then dried over sodium sulfate. Purification be chromatography on an alumina column (eluent: 2% methanol in dichloromethane) makes it possible to obtain 630 mg of the expected compound.

Melting point: 125° C. (Isopropyl ether)

1H NMR (DMSO): 1.40 to 1.68 (unresolved complex, 6); 1.80 (m, 2); 2.07 (m, 2); 2.33 to 2.5 (unresolved complex, 6); 2.53 (t, 2); 3.13 (t, 2); 4.13 (t, 1); 6.23 (d, 1); 7.15 (t, 2); 7.55 to 7.75 (unresolved complex, 4); 7.91 (d, 2); 8.04 (d, 1); 9.47 (s, 1); 9.65 (s, 1).

### Example 118

### 4-({4-[(4-Fluorophenyl)amino]pyrimidin-2-yl}amino)-N-methyl-N-(4-oxocyclohexyl)benzenesulfonamide

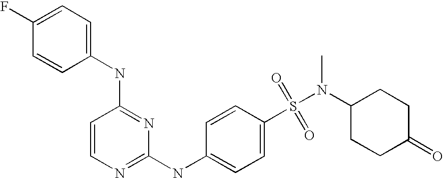

Following the procedure described in example 117, starting with 6.4 g of N-1,4-dioxaspiro[4.5]dec-8-yl-4-({4-[(4-fluorophenyl)amino]pyrimidin-2-yl}amino)-N-methylbenzenesulfonamide, 5.2 g of expected product are obtained.

MH+=470; Melting point: 203° C. (Isopropyl ether)

1H NMR (DMSO): 1.48 (m, 2); 1.76 (qd, 2); 2.06 (bd, 2); 2.50 (m, 2); 2.60 (s, 3); 4.21 (tt, 1); 6.24 (d, 1); 7.13 (t, 2); 7.65 (d, 2); 7.67 (m, 2); 7.93 (d, 2); 8.04 (d, 1); 9.45 (bs, 1); 9.65 (bs, 1).

### Example 119

### 4-({4-[(4-Fluorophenyl)amino]pyrimidin-2-yl}amino)-N-(trans-4-hydroxycyclohexyl)-N-(2-pyrrolidin-1-ylethyl)benzenesulfonamide

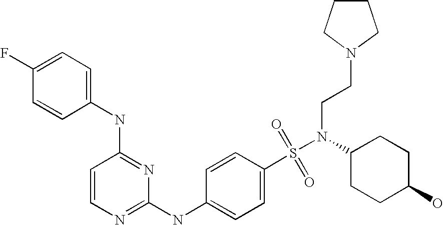

To a solution of 300 mg of 4-({4-[(4-fluorophenyl)-amino]pyrimidin-2-yl}amino)-N-(4-oxocyclohexyl)-N-(2-pyrrolidin-1-ylethyl)benzenesulfonamide (example 117) in 10 ml of methanol are added 40 mg of sodium borohydride and the reaction is kept stirring at room temperature for 3 hours. The medium is taken up in dichloromethane and washed with a sodium carbonate solution. After drying and concentrating to dryness, the crude reaction product is purified by chromatography on an alumina column (eluent: 20% methanol in dichloromethane) makes it possible to obtain 200 mg of the expected compound.

MH+=555.1; Melting point: 135.2° C. (Isopropyl ether)

1H NMR (DMSO): 1-1.8 (unresolved complex, 12); 2.4-2.6 (unresolved complex, 6); 3.1 (m, 2); 3.3 (m, 1); 3.5 (m, 1); 4.5 (d, 1); 6.25 (d, 1); 7.1 (t, 2); 7.6 (unresolved complex, 4); 7.9 (d, 2); 8.1 (d, 1); 9.6 (s, 1); 9.65 (s, 1)

### Example 120

### 4-({4-[(4-Fluorophenyl)amino]pyrimidin-2-yl}amino)-N-(4-hydroxycyclohexyl)-N-methylbenzene-sulfonamide

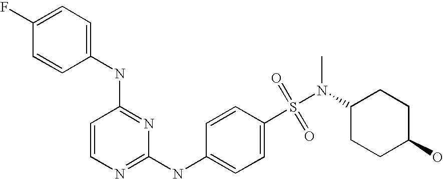

Following the procedure described in example 119, starting with 500 g of 4-({4-[(4-fluorophenyl)-amino]pyrimidin-2-yl}amino)-N-methyl-N-(4-oxocyclo-hexyl)benzenesulfonamide (example 118) 338 g of expected product are obtained.

MH+=472.1; Melting point: 203° C. (Isopropyl ether)

1H NMR (DMSO): 1.70 (unresolved complex, 4); 2.60-2.88 (unresolved complex, 4); 2.74 (s, 3); 3.77 (m, 1); 6.37 (d, 1); 7.26 (t, 2); 7.74 (d, 2); 7.79 (m, 2); 8.04 (d, 2); 8.17 (d, 1); 9.58 (bs, 1); 9.77 (bs, 1).

### Example 121

### 4-({4-[(4-Fluorophenyl)amino]pyrimidin-2-yl}amino)-N-methyl-N-[4-cis(methylamino)cyclohexyl]-benzenesulfonamide

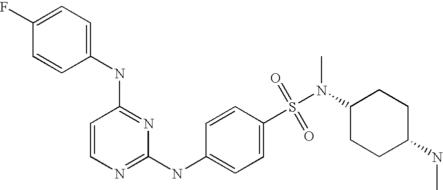

To a solution containing 600 mg of 4-({4-[(4-fluoro-phenyl)amino]pyrimidin-2-yl}amino)-N-methyl-N-(4-oxo-cyclohexyl)benzenesulfonamide, 0.1 ml of acetic acid in 10 ml of a dichloromethane/THF (50/50) mixture are added 1.3 ml of a 2N solution of methylamine in THF, and then 380 mg of sodium triacetoxyborohydride. the reaction medium is kept stirring for 3 hours at room temperature. The medium is taken up in dichloromethane, washed with a sodium carbonate solution and the organic phase is dried over sodium sulfate. After concentrating to dryness, the crude reaction product, containing two isomers (cis and trans), is chromatographed on a silica column (eluent: chloromethane-methanol-aqueous ammonia: 89-10-1). Two fractions are obtained including 283 mg of the expected cis isomer (least polar compound) which crystallizes from isopropyl ether.

MH+=485.1; Melting point: 192° C. (Isopropyl ether)

1H NMR (DMSO): 0.77-1.84 (unresolved complex, 8); 2.57 (s, 3); 3.2 (m, 1); 3.55 (m, 1); 4.49 (d, 1); 6.25 (d, 1); 7.13 (t, 2); 7.59 (d, 2); 7.65 (m, 2); 7.91 (d, 2); 8.05 (d, 1); 9.46 (s, 1); 9.64 (s, 1)

### Example 122

### 4-({4-[(4-Fluorophenyl)amino]pyrimidin-2-yl}amino)-N-methyl-N-[4-trans(methylamino)cyclohexyl]-benzenesulfonamide

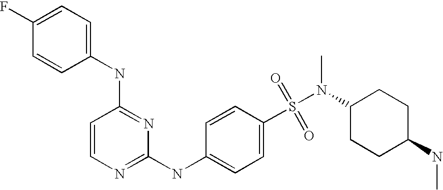

During the chromatography step of example 88, both fractions are obtained including 213 mg of the expected trans isomer (most polar compound) which crystallizes from isopropyl ether.

MH+=485.1; Melting point: 195° C. (Isopropyl ether)

1H NMR (DMSO): 0.94 (m, 2); 1.14-1.65 (unresolved complex, 4); 1.76 (m, 2); 2.04 (m, 1); 2.17 (s, 3); 2.58 (s, 3); 3.57 (m, 1); 6.24 (d, 1); 7.13 (t, 2); 7.59 (d, 2) 7.65 (m, 2); 7.91 (d, 2); 8.04 (d, 1); 9.46 (s, 1); 9.64 (s, 1)

### Example 123

### 4-({4-[(4-Fluorophenyl)amino]pyrimidin-2-yl}amino)-N-(1-methylpiperidin-4-yl)-N-[(2S)-pyrrolidin-2-ylmethyl]benzenesulfonamide

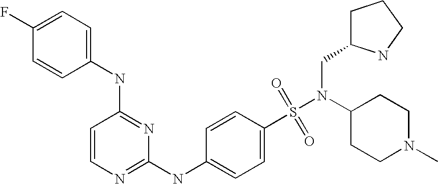

Stage 1: 2-[(1-Methylpiperidin-4-ylamino)methyl]pyrrol-idine-1-carboxylic acid tert-butyl ester: As in example A, starting with 1 g of 1-methylpiperidin-4-one and 2.2 g of 2-S-aminomethylpyrrolidine-1-carboxylic acid tert-butyl ester, 2 g of expected product are obtained [α] (20,589).=+48° (C=0.18, MeOH)

Stage 2: The procedure is carried out as in stage 4 of example 1, starting with 600 mg of 4-({4-[(4-fluoro-phenyl)amino]pyrimidin-2-yl}amino)benzenesulfonyl chloride hydrochloride and 474 mg of 2-[(1-methyl-piperidin-4-ylamino)methyl]pyrrolidine-1-carboxylic acid tert-butyl ester. 300 mg of hydrochloride of the expected product are obtained after a decarboxylation reaction following procedure 2 of example 8 [α] (20,589).=+34° (C=0.11, MeOH).

MH+=540.1; Melting point: 200° C. (Isopropyl ether)

1H NMR (DMSO): 1.4-2.4 (unresolved complex; 8); 2.65-2.9 (2d, 3); 3-4.2 (unresolved complex, 10); 6.5 (d, 1); 7.3 (t, 2); 7.65 (m, 2), 7.85 (d, 2); 7.9 (d, 2); 8.1 (d, 1); 9 (bs, 1); 9.45 (bs, 1); 10.8 (bs, 1); 10.9-11.4 (unresolved complex, 2)

### Example 124

### 4-({4-[(4-Fluorophenyl)amino]pyrimidin-2-yl}amino)-N-(1-methylpiperidin-4-yl)-N-[(2R)-pyrrolidin-2-ylmethyl]benzenesulfonamide

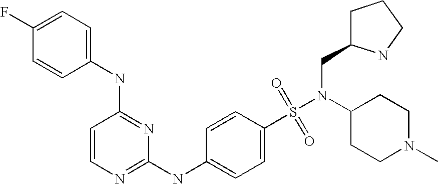

Stage 1: 2-[(1-Methylpiperidin-4-ylamino)methyl]pyrrol-idine-1-carboxylic acid tert-butyl ester: As in example A, starting with 1 g of 1-methylpiperidin-4-one and 1.9 g of 2-R-aminomethylpyrrolidine-1-carboxylic acid tert-butyl ester, 1.9 g of expected product are obtained [α] (20,589).=+48° (C=0.18, MeOH)

Stage 2: The procedure is carried out as in stage 4 of example 1, starting with 600 mg of 4-({4-[(4-fluoro-phenyl)amino]pyrimidin-2-yl}amino)benzenesulfonyl chloride hydrochloride and 474 mg of 2-[(1-methyl-piperidin-4-ylamino)methyl]pyrrolidine-1-carboxylic acid tert-butyl ester. 250 mg of hydrochloride of the expected product are obtained after a decarboxylation reaction following procedure 2 of example 8. [α] (20,589).=−36° (C=0.31, MeOH).

MH+=540.1; Melting point: 200° C. (Isopropyl ether)

1H NMR (DMSO): 1.12-2.26 (unresolved complex, 8); 2.53-2.84 (2d, 3); 2.85-3.59 (unresolved complex, 8); 3.71 (m, 1); 3.95 (m, 1); 6.50 (d, 1); 7.24 (t, 2); 7.54-7.69 (unresolved complex, 2); 7.72-7.92 (unresolved complex, 4); 8.07 (d, 1); 8.90 (bs, 1), 9.40 (bs, 1); 10.70 (bs, 1); 10.98 (bd, 2).

### Example 125

### N-{1-[2-(Ethylthio)ethyl]piperidin-4-yl}-4-({4-[(4-fluorophenyl)amino]pyrimidin-2-yl}amino)-benzenesulfonamide

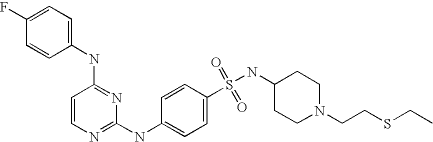

Stage 1: [1-(2-Ethylsulfanylethyl)piperidin-4-yl]carbamic acid tert-butyl ester: A mixture of 6 g of piperidin-4-ylcarbamic acid tert-butyl ester, 4.3 g of 1-bromo-2-ethylsulfanylethane and 6.2 g of potassium carbonate is heated under reflux in 50 ml of ethanol for 48 hours. After filtration of the solid and concentration under vacuum, the medium is taken up in water and extracted with dichloromethane. The organic phase is dried over sodium sulfate. The crude reaction product obtained after concentrating to dryness is chromatographed on a silica column (5% methanol in dichloromethane). 5 g of expected product are thus obtained.

Stage 2: 1-(2-Ethylsulfanylethyl)piperidin-4-ylamine hydrochloride: A decarboxylation reaction following procedure 2 of example 8 makes it possible, starting with 5 g of [1-(2-ethylsulfanylethyl)piperidin-4-yl]carbamic acid tert-butyl ester, to obtain 4.4 g of expected product.

Stage 3: The procedure is carried out as in stage 4 of example 1, starting with 600 mg of 4-({4-[(4-fluoro-phenyl)amino]pyrimidin-2-yl}amino)benzenesulfonyl chloride hydrochloride and 320 mg of 1-(2-ethyl-sulfanylethyl)piperidin-4-ylamine hydrochloride. 190 mg of expected product are thus obtained.

MH+=531.1; Melting point: 194° C. (Isopropyl ether)

1H NMR (DMSO): 1.08 (t, 3); 1.29 (m, 2); 1.46 (m, 2); 1.81 (m, 2); 2.24 to 2.54 (unresolved complex, 6); 2.65 (m, 2); 2.82 (m, 1); 6.23 (dm, 1); 7.13 (t, 2); 7.420 (d, 1); 7.256 to 7.73 (unresolved complex, 4); 7.87 (d, 2); 8.04 (d, 1); 9.47 (s, 1); 9.58 (s, 1).

### Example 126

### 4-({4-[(4-Fluorophenyl)amino]pyrimidin-2-yl}amino)-N-{1-[2-(methylsulfonyl)ethyl]piperidin-4-yl}benzenesulfonamide

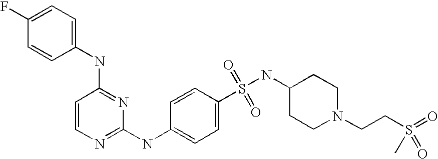

Stage 1: [1-(2-Methanesulfonylethyl)piperidin-4-yl]car-bamic acid tert-butyl ester: To a solution of 4.3 g of piperidin-4-ylcarbamic acid tert-butyl ester, 10.3 g of triethylamine in 50 ml of ethanol are added dropwise 5.4 g of methanesulfonylethene and the medium is kept stirring at room temperature for 4 hours. The medium is concentrated under vacuum and purified by chromatography on a silica column (6% methanol in dichloromethane). 5 g of expected product are thus obtained.

Stage 2: 1-(2-Methanesulfonylethyl)piperidin-4-ylamine hydrochloride: 5 g of [1-(2-methanesulfonylethyl)-piperidin-4-yl]carbamic acid tert-butyl ester in solution in 10 ml of dioxane are treated with 15 ml of a 2M solution of hydrogen chloride in dioxane. The reaction medium is kept stirring for 18 hours. The medium is concentrated under vacuum and taken up in isopropyl ether before the filtration step which makes it possible to obtain 4.3 g of expected compound.

Stage 3: The procedure is carried out as in stage 4 of example 1 starting with 1 g of 4-({4-[(4-fluorophenyl)-amino]pyrimidin-2-yl}amino)benzenesulfonyl chloride hydrochloride which is reacted with 740 mg of 1-(2-methanesulfonylethyl)piperidin-4-ylamine hydrochloride obtained in stage 2. 180 mg of expected product are thus obtained.

MH+=549; Melting point: 183° C. (Isopropyl ether)

1H NMR (DMSO): 1.28 (m, 2); 1.46 (m, 2); 1.86 (m, 2); 2.55 (t, 2); 2.66 (m, 2); 2.83 (m, 1); 2.91 (s, 3); 3.14 (t, 2); 6.23 (d, 1); 7.13 (t, 2); 7.42 (d, 1); 7.54 to 7.72 (unresolved complex, 4); 7.87 (d, 2); 8.04 (d, 1); 9.43 (s, 1); 9.59 (s, 1).

### Example 127

### 4-[[4-[(4-Fluorophenyl)amino]-2-pyrimidin-yl]amino]-N-(2-methoxyethyl)-N-(tetrahydro-1,1-dioxido-3-thienyl)benzenesulfonamide

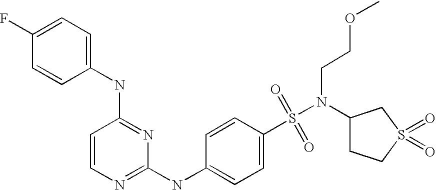

The procedure is carried out as in stage 4 of example 1, starting with 600 mg of 4-({4-[(4-fluoro-phenyl)amino]pyrimidin-2-yl}amino)benzenesulfonyl chloride hydrochloride which are reacted with 400 mg of (tetrahydro-1,1-dioxido-3-thienyl)(2-methoxyethyl)amine. 30 mg of expected product are thus obtained.

MH+=536.1; Melting point: 150° C. (Isopropyl ether)

1H NMR (DMSO): 2.09 (m, 2); 2.87 (dd, 1); 2.98-3.11 (unresolved complex, 2); 3.20 (m, 1); 3.24 (m, 2); 3.26 (s, 3); 3.51 (t, 2); 4.67 (qui, 1); 6.31 (d, 1); 7.20 (t, 2); 7.66-7.78 (unresolved complex, 4); 8.00 (d, 2); 8.10 (d, 1); 9.53 (bs, 1); 9.76 (bs, 1).

### Example 128

### 4-({4-[(4-Fluorophenyl)amino]pyrimidin-2-yl}amino)-N-allyl-N-(1-methylpiperidin-4-yl)benzene-sulfonamide

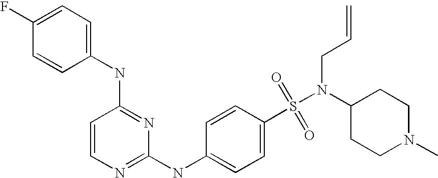

The procedure is carried out as in stage 4 of example 1, starting with 600 mg of 4-({4-[(4-fluoro-phenyl)amino]pyrimidin-2-yl}amino)benzenesulfonyl chloride hydrochloride which are reacted with 245 mg of allyl-(1-methylpiperidin-4-yl)amine. 260 mg of expected product are thus obtained.

MH+=497.1; Melting point: 167° C. (Isopropyl ether)

1H NMR (DMSO): 1.32 (m, 2); 1.65 (m, 2); 1.83 (m, 2); 2.09 (s, 3); 2.71 (m, 2); 3.55 (m, 1); 3.81 (d, 2); 5.09 (d, 1); 5.22 (d, 1); 5.79 (m, 1); 6.29 (d, 1); 7.17 (t, 2); 7.66 (m, 2); 7.69 (m, 2); 7.92 (m, 2); 8.08 (d, 1); 9.48 (s, 1); 9.66 (s, 1).

### Example 129

### 4-({4-[(4-Fluoro-3-methylphenyl)amino]-pyrimidin-2-yl}amino)-N-methyl-N-{1-[2-(methyl-sulfonyl)ethyl]piperidin-4-yl}benzenesulfonamide

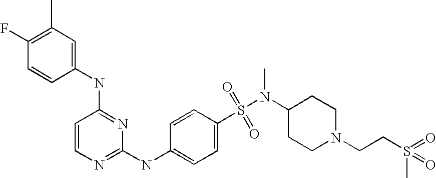

500 mg of 4-({4-[(4-fluoro-3-methylphenyl)amino]pyrim-idin-2-yl}amino)-N-methyl-N-piperidin-4-ylbenzenesul-fonamide hydrochloride obtained in example 106 are mixed with 10 ml of a methanol-dichloromethane (4-1) solution. 400 mg of triethylamine are added, followed by 160 mg of methyl vinyl sulfone. The reaction medium is kept stirring for 3 hours at room temperature. After concentrating to dryness, taking up in dichloromethane and washing with a sodium carbonate solution, the organic phase is dried and concentrated under vacuum. Purification by column chromatography (eluent: dichloromethane-methanol 95-5) makes it possible to obtain 240 mg of desired compound which crystallizes from an isopropyl ether-dichloromethane mixture.

MH+=577.1; Melting point: 114.5° C. (Isopropyl ether-dichloromethane)

1H NMR (DMSO): 1.22 (d, 2); 1.54 (q, 2); 1.96 (t, 2); 2.25 (s, 3); 2.57-2.72 (unresolved complex, 5); 2.86 (d, 2); 2.97 (s, 3); 3.21 (t, 2); 3.62 (m, 1); 6.28 (d, 1); 7.11 (t, 1); 7.46 (m, 1); 7.54-7.70 (unresolved complex, 3); 7.96 (d, 2); 8.08 (d, 1); 9.43 (bs, 1); 9.69 (bs, 1).

### Example 130

### 4-({4-[(4-Fluorophenyl)amino]pyrimidin-2-yl}amino)-N-propyl-N-(1-methylpiperidin-4-yl)benzene-sulfonamide

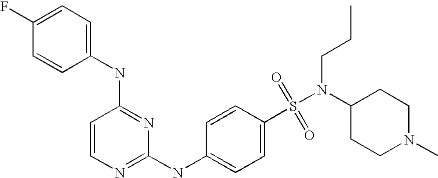

Stage 1: (1-Methylpiperidin-4-yl)propylamine: As in example A, starting with 2 g of 1-methylpiperidin-4-one and 1.2 g of 2-propylamine, 1.38 g of expected product are obtained.

Stage 2: The procedure is carried out as in stage 4 of example 1, starting with 600 mg of 4-({4-[(4-fluoro-phenyl)amino]pyrimidin-2-yl}amino)benzenesulfonyl chloride hydrochloride which are reacted with 250 mg of (1-methylpiperidin-4-yl)propylamine. 130 mg of expected product are thus obtained.

MH+=499.1; Melting point: 232° C. (Isopropyl ether)

### Example 131

### 4-({4-[(4-Fluoro-3-methylphenyl)amino]-pyrimidin-2-yl}amino)-N-methyl-N-{1-[(1-thiazol-2-yl)-methyl]piperidin-4-yl}benzenesulfonamide

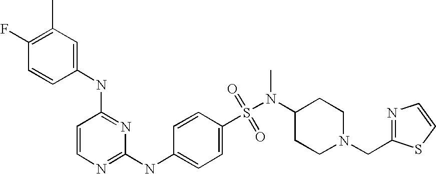

As in example A, starting with 360 mg of regenerated 4-({4-[(4-fluoro-3-methylphenyl)amino]pyrimidin-2-yl}-amino)-N-methyl-N-piperidin-4-ylbenzenesulfonamide (example 106) and 90 mg of thiazole-4-carbaldehyde, 260 mg of expected product are obtained.

MH+=568.1; Melting point=176-177° C. (Isopropyl ether/dichloromethane)

1H NMR (DMSO): 1.20 (d, 2); 1.57 (m, 2); 1.99 (t, 2); 2.23 (d, 3); 2.64 (s, 3); 2.82 (d, 2); 3.47-3.71 (unresolved complex, 3); 6.27 (d, 1); 7.10 (t, 1); 7.39-7.51 (unresolved complex, 2); 7.52-7.67 (unresolved complex, 3); 7.93 (d, 2); 8.06 (d, 1), 9.01 (d, 1); 9.41 (bs, 1); 9.67 (bs, 1).

### Example 132

### 4-({4-[(4-Fluoro-3-methylphenyl)amino]-pyrimidin-2-yl}amino)-N-methyl-N-{1-[(1-thiazol-5-yl)-methyl]piperidin-4-yl}benzenesulfonamide

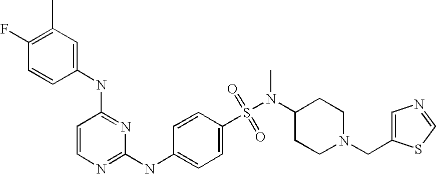

As in example A, starting with 360 mg of regenerated 4-({4-[(4-fluoro-3-methylphenyl)amino]pyrimidin-2-yl}-amino)-N-methyl-N-piperidin-4-ylbenzenesulfonamide obtained in example 106 and 90 mg of thiazole-5-carbaldehyde, 182 mg of expected product are obtained.

MH+=568.1; Melting point=196-197° C. (Isopropyl ether/dichloromethane)

1H NMR (DMSO): 1.22 (d, 2); 1.55 (m, 2); 1.97 (t, 2); 2.23 (d, 3); 2.64 (s, 3); 2.78 (d, 2); 3.61 (m, 1); 3.68 (s, 2), 6.27 (d, 1); 7.10 (t, 1); 7.45 (m, 1); 7.52-7.67 (unresolved complex, 3); 7.72 (s, 1); 7.93 (d, 2); 8.06 (d, 1), 9 (s, 1); 9.41 (bs, 1); 9.67 (bs, 1).

### Example 133

### 4-({4-[(4-Fluoro-3-methylphenyl)amino]-pyrimidin-2-yl}amino)-N-methyl-N-(1-{[1-(1,2,3)thia-diazol-4-yl]methyl}piperidin-4-yl)benzenesulfonamide

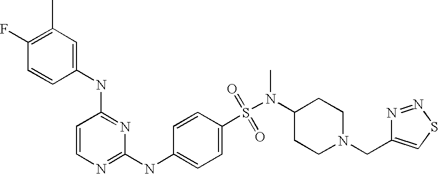

As in example A, starting with 450 mg of regenerated 4-({4-[(4-fluoro-3-methylphenyl)amino]pyrimidin-2-yl}-amino)-N-methyl-N-piperidin-4-ylbenzenesulfonamide obtained in example 106 and 110 mg of 1,2,3-thiadiazole-4-carbaldehyde, 296 mg of expected product are obtained.

MH+=568.1; Melting point=168° C. (Isopropyl ether/dichloromethane)

1H NMR (DMSO): 1.18 (m, 2); 1.53 (m, 2); 2.00 (m, 2); 2.19 (s, 3); 2.29 (s, 3); 2.79 (m, 2); 3.56 (m, 1); 3.94 (s, 2); 6.247 (d, 1); 7.05 (t, 1); 7.41 (m, 1); 7.48 to 7.63 (unresolved complex, 3); 7.88 (d, 2); 8.026 (d, 1); 8.96 (s, 1); 9.37 (s, 1); 9.61 (s, 1); 12.6 (s, 1)

### Example 134

### 4-({4-[(4-Fluoro-3-methylphenyl)amino]-pyrimidin-2-yl}amino)-N-methyl-N-{1-[(1H-pyrazol-4-yl)methyl]piperidin-4-yl}benzenesulfonamide

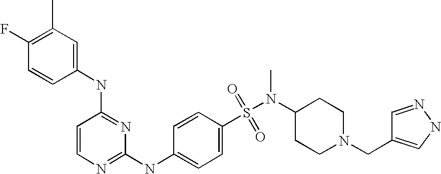

As in example A, starting with 450 mg of regenerated 4-({4-[(4-fluoro-3-methylphenyl)amino]pyrimidin-2-yl}-amino)-N-methyl-N-piperidin-4-ylbenzenesulfonamide obtained in example 106 and 100 mg of 1H-pyrazole-4-carbaldehyde, 228 mg of expected product are obtained.

MH+=551.2; Melting point=224° C. (Isopropyl ether/dichloromethane)

1H NMR (DMSO): 1.25 (m, 2); 1.54 (m, 2); 1.85 (t, 2); 2.24 (s, 3); 2.65 (s, 3); 2.77 (m, 2); 3.30 (s, 2); 3.57 (m, 1); 6.27 (d, 1); 7.10 (t, 1); 7.21 to 7.67 (unresolved complex, 6); 7.93 (d, 2); 8.06 (d, 1); 9.41 (s, 1); 9.66 (s, 1); 12.6 (s, 1)

### Example 135

### 4-({4-[(4-Fluoro-3-methylphenyl)amino]-pyrimidin-2-yl}amino)-N-methyl-N-{1-[(1-methyl-1H-pyrazol-4-yl)methyl]piperidin-4-yl}benzenesulfonamide

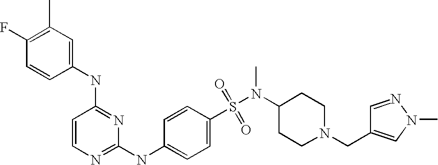

As in example A, starting with 450 mg of regenerated 4-({4-[(4-fluoro-3-methylphenyl)amino]pyrimidin-2-yl}-amino)-N-methyl-N-piperidin-4-ylbenzenesulfonamide obtained in example 106 and 110 mg of 1-methyl-1H-pyrazole-4-carbaldehyde, 154 mg of expected product are obtained.

MH+=565.2; Melting point=125-150° C. (Isopropyl ether/dichloromethane)

1H NMR (DMSO): 1.21 (d, 2); 1.54 (qd, 2); 1.86 (t, 2); 2.24 (s, 3); 2.64 (s, 3); 2.77 (d, 2); 3.27 (s, 2); 3.59 (m, 1); 3.76 (s, 3); 6.28 (d, 1); 7.11 (t, 1); 7.24 (s, 1); 7.46 (m, 1); 7.51 (s, 1); 7.58 (dd, 1); 7.62 (d, 2); 7.94 (d, 2); 8.07 (d, 1); 9.41 (s, 1); 9.67 (s, 1).

### Example 136

### 4-({4-[(4-Fluoro-3-methylphenyl)amino]-pyrimidin-2-yl}amino)-N-methyl-N-{1-[(5-methyl-2H-pyrazol-3-yl)methyl]piperidin-4-yl}benzenesulfonamide

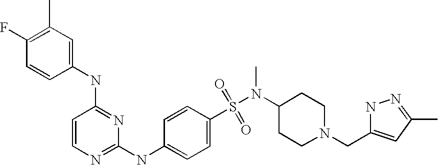

As in example A, starting with 450 mg of regenerated 4-({4-[(4-fluoro-3-methylphenyl)amino]pyrimidin-2-yl}-amino)-N-methyl-N-piperidin-4-ylbenzenesulfonamide obtained in example 106 and 110 mg of 5-methyl-2H-pyrazole-3-carbaldehyde, 136 mg of expected product are obtained.

MH+=564.2; Melting point=130° C. (Isopropyl ether/dichloromethane)

1H NMR (DMSO): 1.21 (d, 2); 1.55 (q, 2); 1.90 (t, 2); 2.14 (s, 3); 2.24 (s, 3); 2.65 (s, 3); 2.76 (d, 2); 3.31 (bs, 2); 3.55 (m, 1); 5.83 (s, 1); 6.28 (d, 1); 7.11 (t, 1); 7.46 (m, 1); 7.58 (dd, 1); 7.62 (d, 2); 7.94 (d, 2); 8.08 (d, 1); 9.41 (s, 1); 9.67 (bs, 1); 12.17 (s, 1).

### Example 137

### N-(4,4-Difluorocyclohexyl)-4-[4-(4-fluoro-3-methylphenylamino)pyrimidin-2-ylamino]-N-methyl-benzenesulfonamide

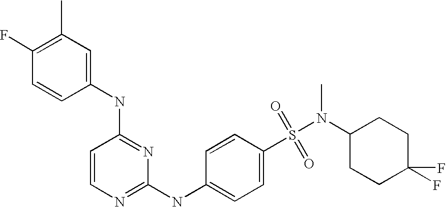

Stage 1: tert-Butoxy-(4,4-difluorocyclohexylamino)-methanol: 2.5 g of 4,4-difluorocyclohexylamine hydrochloride in the presence of 3.2 g of (t-BuOCO)2O and 2.5 ml of triethylamine in 50 ml of dichloromethane. After one night at room temperature, the reaction medium is left at 50° C. for 5 hours. The medium is evaporated to dryness and then extracted with ethyl acetate. The organic phase is washed with a saturated NaCl solution and then dried over sodium sulfate. 3.3 g of expected product are obtained after chromatography (dichloromethane as a gradient up to 1% methanol) on silica.

Stage 2: tert-Butoxy-[(4,4-difluorocyclohexyl)methyl-amino]methanol: 1 g of tert-butoxy-(4,4-difluorocyclo-hexylamino)methanol obtained in stage 2 is added to a solution, kept at 0° C., containing 190 mg of sodium hydride (60%) in 40 ml of tetrahydrofuran. The reaction medium is kept stirring at this temperature for 30 minutes. 0.3 ml of methyl iodide is added and then the medium is kept stirring at room temperature overnight. After the usual treatment, 400 mg of expected product are obtained.

Stage 3: (4,4-Difluorocyclohexyl)methylamine hydro-chloride: Following the decarboxylation reaction described in procedure 2 of example 8, 300 mg of expected product are obtained from 400 mg of tert-butoxy-[(4,4-difluorocyclohexyl)methylamino]methanol obtained in stage 3.

Stage 4: The procedure is carried out as in stage 4 of example 1, starting with 600 mg of 4-({4-[(4-fluoro-3-methylphenyl)amino]pyrimidin-2-yl}amino)benzenesulfonyl chloride hydrochloride and 300 mg of (4,4-difluoro-cyclohexyl)methylamine. 367 mg of expected product are obtained.

MH+=505.1; Melting point=164-165° C. (Isopropyl ether/dichloromethane)

1H NMR (DMSO): 1.38 (m, 2); 1.78 to 2.12 (unresolved complex, 4); 2.25 (s, 3); 2.65 (s, 3); 3.92 (m, 1); 6.28 (d, 1); 7.10 (t, 1); 7.46 (m, 1); 7.55 (m, 1); 7.66 (d, 2); 7.95 (d, 2); 8.07 (d, 1); 9.41 (s, 1); 9.67 (s, 1).

### Example 138

### 4-({4-[(4-Fluoro-3-methylphenyl)amino]-pyrimidin-2-yl}amino)-N-methyl-N-{1-[(5-methylisoxazol-3-yl)methyl]piperidin-4-yl}benzenesulfonamide

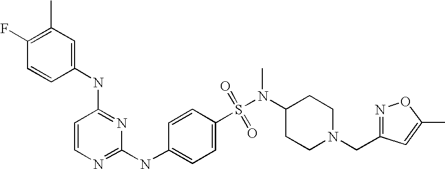

As in example A, starting with 200 mg of regenerated 4-({4-[(4-fluoro-3-methylphenyl)amino]pyrimidin-2-yl}-amino)-N-methyl-N-piperidin-4-ylbenzenesulfonamide obtained in example 106 and 50 mg of 5-methyl-3-isoxazolecarbaldehyde, 148 mg of expected product are obtained.

MH+=566.3; Melting point=172-173° C. (Isopropyl ether/dichloromethane)

1H NMR (DMSO): 1.24 (d, 2); 1.57 (qd, 2); 2.01 (t, 2); 2.24 (s, 3); 2.36 (s, 3); 2.65 (s, 3); 2.76 (d, 2); 3.44 (s, 2); 3.61 (m, 1); 6.12 (s, 1); 6.27 (d, 1); 7.10 (t, 1); 7.45 (m, 1); 7.57 (d, 1); 7.62 (d, 2); 7.94 (d, 2); 8.07 (d, 1); 9.40 (s, 1); 9.67 (s, 1).

### Example 139

### 4-({4-[(4-Fluoro-3-methylphenyl)amino]-pyrimidin-2-yl}amino)-N-methyl-N-(1-methylpiperidin-3-yl)benzenesulfonamide

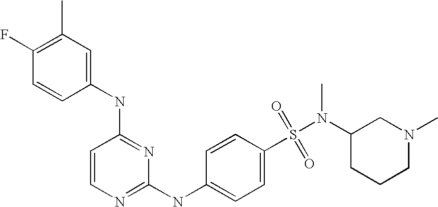

Stage 1: 4-({4-[(4-Fluoro-3-methylphenyl)amino]pyrimidin-2-yl}amino)-N-methyl-N-(1-H-piperidin-3-yl)-benzenesulfonamide hydrochloride: The procedure is carried out as in stage 4 of example 1, starting with 800 mg of 4-[4-(3-methyl-4-fluorophenylamino)pyrimidin-2-ylamino]benzenesulfonyl chloride hydrochloride which are reacted with 485 mg of 3-methylaminopiperidine-1-carboxylic acid tert-butyl ester. 320 mg of expected product are thus obtained after an additional step using a decarboxylation reaction following procedure 2 of example 8.

Stage 2: As in example A, starting with 320 mg of 4-({4-[(4-fluoro-3-methylphenyl)amino]pyrimidin-2-yl}amino)-N-methyl-N-(1-H-piperidin-3-yl)benzenesulfonamide obtained in stage 1 and 0.06 ml of formaldehyde, 142 mg of expected product are obtained.

MH+=485.2; Melting point=188° C. (Isopropyl ether/dichloromethane)

1H NMR (DMSO): 1.15 to 1.72 (unresolved complex, 5); 1.848 (t, 1); 2.08 (s, 3); 2.25 (s, 3); 2.46 (m, 1); 2.60 (bd, 1); 2.68 (s, 3); 3.72 (m, 1); 6.28 (d, 1); 7.11 (t, 1); 7.47 (m, 1); 7.58 (m, 1); 7.63 (d, 2); 7.96 (d, 2); 8.08 (d, 1); 9.44 (s, 1); 9.70 (s, 1).

### Example 140

### 4-({4-[(4-Fluorophenyl)amino]pyrimidin-2-yl}amino)-N-methyl-N-[4-cis(dimethylamino)cyclohexyl]-benzenesulfonamide

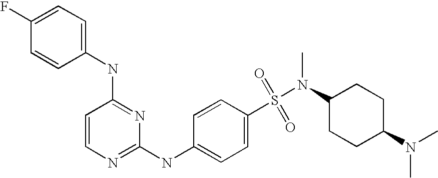

Following the procedure described in example 121, starting with 600 mg of 4-({4-[(4-fluorophenyl)amino]-pyrimidin-2-yl}amino)-N-methyl-N-(4-oxocyclohexyl)-benzenesulfonamide (example 118) and 1.28 ml of dimethylamine in solution in THF, two fractions are obtained including 283 mg of the expected cis isomer (least polar compound) 4-({4-[(4-fluorophenyl)amino]-pyrimidin-2-yl}amino)-N-methyl-N-[4-cis(dimethyl-amino)cyclohexyl]benzenesulfonamide.

MH+=499.1; Melting point: 222° C. (Isopropyl ether)

1H NMR (DMSO): 1.02 (m, 2); 1.32 (m, 2); 1.64 (m, 2); 1.80-1.94 (unresolved complex, 3); 2.07 (s, 6); 2.64 (s, 3); 3.72 (m, 1); 6.30 (d, 1); 7.17 (t, 2); 7.63 (d, 2); 7.70 (m, 2); 7.94 (d, 2); 8.01 (d, 1); 9.48 (s, 1); 9.66 (s, 1)

### Example 141

### 4-({4-[(4-Fluorophenyl)amino]pyrimidin-2-yl}amino)-N-methyl-N-[4-trans(dimethylamino)cyclo-hexyl]benzenesulfonamide

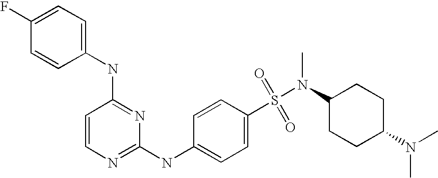

During the chromatography step of example 140, both fractions are obtained including 290 mg of the expected trans isomer (most polar compound) which crystallizes from isopropyl ether.

MH+=499.1; Melting point: 218° C. (Isopropyl ether)

1H NMR (DMSO): 1.08-1.5 (unresolved complex, 6); 1.72 (m, 2); 2.03 (m, 1); 2.10 (s, 6); 2.63 (s, 3); 3.6 (m, 1); 6.29 (d, 1); 7.17 (t, 2); 63 (d, 2); 7.71 (m, 2); 7.94 (d, 2); 8.08 (d, 1); 9.49 (s, 1); 9.57 (s, 1)

### Example 142

### 4-({4-[(4-Fluorophenyl)amino]pyrimidin-2-yl}amino)-N-methyl-N-[4-cis(ethylamino)cyclohexyl]-benzenesulfonamide

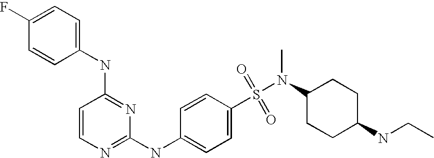

Following the procedure described in example 121, starting with 600 mg of 4-({4-[(4-fluorophenyl)amino]-pyrimidin-2-yl}amino)-N-methyl-N-(4-oxocyclohexyl)-benzenesulfonamide (example 118) and 1.28 ml of ethyl-amine in solution in THF, both fractions are obtained including 310 mg of the expected cis isomer (least polar compound) 4-({4-[(4-fluorophenyl)amino]pyrimidin-2-yl}amino)-N-methyl-N-[4-cis(ethylamino)cyclohexyl]-benzenesulfonamide.

MH+=499.1; Melting point: 188° C. (Isopropyl ether)

1H NMR (DMSO): 0.82-1.08 (unresolved complex, 5); 1.28 (ls, 1); 1.37 (m, 2); 1.67 (m, 4); 2.41 (q, 2); 2.63 (m, 1); 2.66 (s, 3); 3.62 (m, 1); 6.28 (d, 1); 7.17 (t, 2); 7.62 (d, 2); 7.70 (m, 2); 7.94 (d, 2); 8.08 (d, 1); 9.49 (s, 1); 9.66 (s, 1).

### Example 143

### 4-({4-[(4-Fluorophenyl)amino]pyrimidin-2-yl}amino)-N-methyl-N-[4-trans(ethylamino)cyclohexyl]-benzenesulfonamide

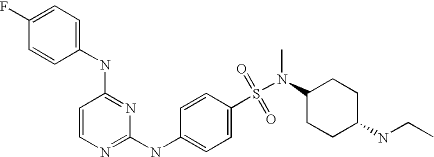

During the chromatography step of example 142, both fractions are obtained including 171 mg of the expected trans isomer (most polar compound) which crystallizes from isopropyl ether.

MH+=499.1; Melting point: 178° C. (Isopropyl ether)

1H NMR (DMSO): 0.85-1.15 (unresolved complex, 5); 1.19-1.51 (unresolved complex, 4); 1.82 (m, 2); 2.2 (m, 1); 2.48 (q, 2); 2.64 (s, 3); 3.6 (m, 1); 6.29 (d, 1); 7.17 (t, 2); 7.62 (d, 2); 7.70 (m, 2); 7.96 (d, 2); 8.08 (d, 1); 9.48 (s, 1); 9.66 (s, 1)

### Example 144

### 4-({4-[(4-Fluorophenyl)amino]pyrimidin-2-yl}amino)-N-methyl-N-[4-cis(2-methylsulfanylethyl-amino)cyclohexyl]benzenesulfonamide

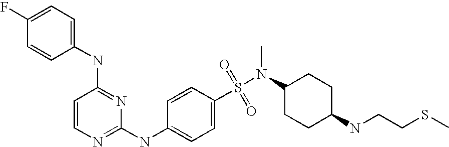

Following the procedure described in example 121, starting with 600 mg of 4-({4-[(4-fluorophenyl)amino]-pyrimidin-2-yl}amino)-N-methyl-N-(4-oxocyclohexyl)-benzenesulfonamide (example 118) and 250 mg of 2-methylsulfanylethylamine, both fractions are obtained including 280 mg of the expected cis isomer (least polar compound) 4-({4-[(4-fluorophenyl)amino]pyrimidin-2-yl}amino)-N-methyl-N-[4-cis(2-methylsulfanylethyl-amino)cyclohexyl]benzenesulfonamide.

MH+=545.3; Melting point: 146° C. (Isopropyl ether)

1.01 (q, 2); 1.28 (m, 2); 1.40 (q, 2); 1.53 (bs, 1); 1.81 (d, 2); 2.02 (s, 3); 2.24 (m, 1); 2.49 (t, 2); 2.63 (s, 3); 2.65 (t, 2); 3.62 (m, 1); 6.29 (d, 1); 7.17 (t, 2); 7.63 (d, 2); 7.70 (dd, 2); 7.94 (d, 2); 8.08 (d, 1); 9.49 (s, 1); 9.67 (s, 1).

### Example 145

### 4-({4-[(4-Fluorophenyl)amino]pyrimidin-2-yl}amino)-N-methyl-N-[4-trans(2-methylsulfanylethyl-amino)cyclohexyl]benzenesulfonamide

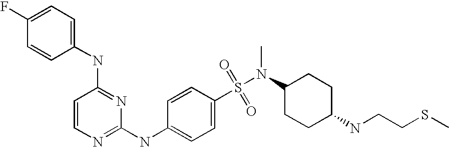

During the chromatography step of example 144, both fractions are obtained including 230 mg of the expected trans isomer (most polar compound) which crystallizes from isopropyl ether.

MH+=545.3; Melting point: 171° C. (Isopropyl ether)

1H NMR (DMSO): 1.00 (d, 2); 1.40 (t, 2); 1.52 (s, 1); 1.66 (m, 4); 2.03 (s, 3); 2.51 (t, 2); 2.59 (m, 2); 2.66 (s, 3); 2.67 (m, 1); 3.63 (m, 1); 6.29 (d, 1); 7.17 (t, 2); 7.63 (d, 2); 7.70 (m, 2); 7.94 (d, 2); 8.08 (d, 1); 9.48 (s, 1); 9.66 (s, 1).

### Example 146

### 4-({4-[(4-Fluorophenyl)amino]pyrimidin-2-yl}amino)-N-(4-cis(methylamino)cyclohexyl)-N-(2-pyrrolidin-1-ylethyl)benzenesulfonamide

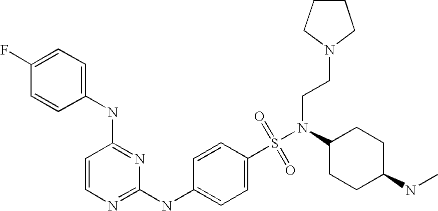

Following the procedure described in example 121, starting with 600 mg of 4-({4-[(4-fluorophenyl)amino]-pyrimidin-2-yl}amino)-N-(4-oxocyclohexyl)-N-(2-pyrrol-idin-1-ylethyl)benzenesulfonamide (example 117) and 1 ml of a 2N solution of methylamine, both fractions are obtained including 260 mg of the expected cis isomer (least polar compound) 4-({4-[(4-fluorophenyl)-amino]pyrimidin-2-yl}amino)-N-(4-cis(methylamino)-cyclohexyl)-N-(2-pyrrolidin-1-ylethyl)benzenesulfonamide.

MH+=568.3; Melting point: 189° C. (Isopropyl ether)

1H NMR (DMSO): 1.1 (d, 2); 1.35 (t, 2); 1.49 (bs, 1); 1.60-1.74 (m, 8); 2.17 (s, 3); 2.45 (m, 4); 2.50 (m, 1); 2.58 (m, 2); 3.17 (m, 2); 3.53 (td, 1); 6.28 (d, 1); 7.71 (t, 2); 7.67 (d, 2); 7.70 (dd, 2); 7.92 (d, 2); 8.08 (d, 1); 9.48 (s, 1); 9.66 (s, 1).

### Example 147

### 4-({4-[(4-Fluorophenyl)amino]pyrimidin-2-yl}amino)-N-(4-trans(methylamino)cyclohexyl)-N-(2-pyr-rolidin-1-ylethyl)benzenesulfonamide

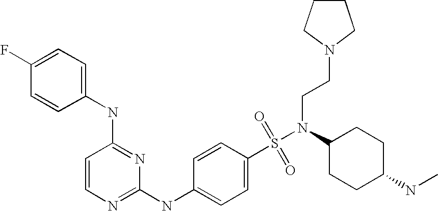

During the chromatography step of example 146, both fractions are obtained including 89 mg of the expected trans isomer (most polar compound) which crystallizes from isopropyl ether.

MH+=568.3; Melting point: 128° C. (Isopropyl ether)

1H NMR (DMSO): 0.97 (m, 2); 1.41 (m, 4); 1.66 (m, 4); 1.84 (d, 2); 2.13 (m, 1); 2.21 (s, 3); 2.45 (t, 4); 2.578 (m, 2); 3.15 (m, 2); 3.52 (m, 1); 6.29 (d, 1); 7.17 (t, 2); 7.66 (d, 2); 7.70 (dd, 2); 7.93 (d, 2); 8.08 (d, 1); 9.49 (s, 1); 9.66 (s, 1).

### Example 148

### 4-({4-[(4-Fluorophenyl)amino]pyrimidin-2-yl}amino)-N-propargyl-N-(1-methylpiperidin-4-yl)-benzenesulfonamide

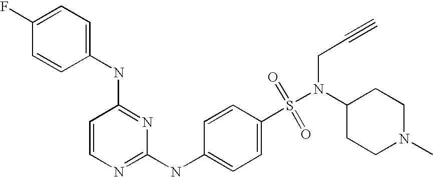

Stage 1: (1-Methylpiperidin-4-yl)propargylamine: As in example A, starting with 2 g of 1-methylpiperidin-4-one and 1.15 g of 2-propargylamine, 2.3 g of expected product are obtained.

Stage 2: The procedure is carried out as in stage 4 of example 1 starting with 600 mg of 4-({4-[(4-fluoro-phenyl)amino]pyrimidin-2-yl}amino)benzenesulfonyl chloride hydrochloride and 250 mg of (1-methylpiperidin-4-yl)propargylamine. 80 mg of expected product are thus obtained.

MH+=495.2; Melting point: 187° C. (Isopropyl ether)

1H NMR (DMSO): 1.35 (d, 2); 1.65-1.95 (m, 4); 2.08 (s, 3); 2.72 (d, 2); 3.11 (s, 1); 3.49 (m, 1); 4.02 (d, 2); 6.24 (d, 1); 7.13 (t, 2); 7.60-7.70 (m, 4); 7.89 (d, 2); 8.04 (d, 1); 9.45 (bs, 1); 9.63 (bs, 1).

### Example 149

### 4-({4-[(4-Fluorophenyl)amino]pyrimidin-2-yl}amino)-N-ethyl-N-(1-methylpiperidin-4-yl)benzene-sulfonamide

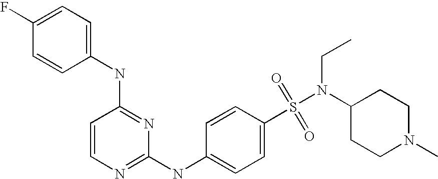

Stage 1: Benzylethyl(1-methylpiperidin-4-yl)amine: As in example A, starting with 2 g of 1-methylpiperidin-4-one and 2.8 g of N-benzylethylamine, 1.3 g of expected product are obtained.

Stage 2: Following the process described in stage 2 of example 30, 700 mg of ethyl(1-methylpiperidin-4-yl)amine are obtained from 1.7 g of benzylethyl(1-methylpiperidin-4-yl)amine.

Stage 3: The procedure is carried out as in stage 4 of example 1, starting with 600 mg of 4-({4-[(4-fluoro-phenyl)amino]pyrimidin-2-yl}amino)benzenesulfonyl chloride hydrochloride which are reacted with 225 mg of ethyl(1-methylpiperidin-4-yl)amine. 100 mg of expected product are thus obtained.

MH+=485.1; Melting point: 154° C. (Isopropyl ether)

1H NMR (DMSO): 1.14 (t, 3); 1.35 (d, 2); 1.62 (qd, 2); 1.86 (t, 2); 2.11 (s, 3); 2.73 (d, 2); 3.16 (q, 2); 3.51 (m, 1); 6.28 (d, 1); 7.17 (t, 2); 7.65 (d, 2); 7.70 (dd, 2); 7.92 (d, 2); 8.08 (d, 1); 9.48 (s, 1); 9.65 (s, 1).

### Example 150

### N-(1-Cyclopropylpiperidin-4-yl)-4-({4-[(4-fluorophenyl)amino]pyrimidin-2-yl}amino)-N-methyl-benzenesulfonamide

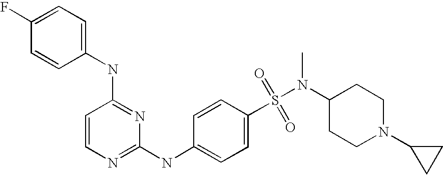

500 mg of 4-({4-[(4-fluorophenyl)amino]pyrimidin-2-yl}-amino)-N-methyl-N-piperidin-4-ylbenzenesulfonamide hydrochloride (example 8) are reacted with 1.4 g of (1-ethoxycyclopropoxy)trimethylsilane in the presence of 530 mg of sodium cyanoborohydride in 20 ml of methanol. The reaction medium is left at 60° C. for 38 hours. After concentrating to dryness, taken up in a 10% sodium carbonate solution, the medium is extracted three times with dichloromethane. The organic phase is dried over magnesium sulfate and concentrated to dryness. 280 mg of expected product are obtained after chromatography on a silica column.

MH+=497.1; Melting point=125-128° C. (Isopropyl ether/dichloromethane)

1H NMR (DMSO): 1.18 (d, 2); 1.54 (m, 2); 1.96 (t, 2); 2.30 to 2.48 (unresolved complex, 4); 2.65 (s, 3); 2.84 (d, 2); 3.63 (m, 1); 6.27 (d, 1); 7.19 (t, 2); 7.66 (d, 2); 7.72 (dd, 2); 7.93 (d, 2); 8.07 (d, 1); 9.51 (bs, 1); 9.69 (bs, 1).

### Example 151

### N-(5-Aminopentyl)-4-({2-[(4-fluorophenyl)-amino]pyrimidin-4-yl}amino)-N-piperidin-4-ylbenzene-sulfonamide hydrochloride

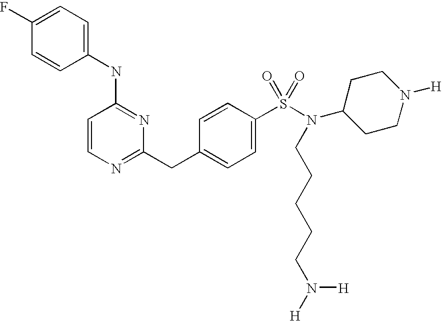

Stage 1: 4-(3-tert-Butoxycarbonylaminopentylamino)-piperidine-1-carboxylic acid tert-butyl ester: As in example A, starting with 3 g of 4-oxopiperidine-1-carboxylic acid tert-butyl ester and 2.97 g of (3-aminopentyl)carbamic acid tert-butyl ester, 4 g of expected product are obtained.

Stage 2: N-(3-Aminopentyl)-4-({4-[(4-fluorophenyl)-amino]pyrimidin-2-yl}amino)-N-piperidin-4-ylbenzene-sulfonamide hydrochloride: The procedure is carried out as in stage 4 of example 1 starting with 600 mg of 4-[4-(4-fluorophenylamino)pyrimidin-2-ylamino]benzenesulfonyl chloride hydrochloride and 600 mg of 4-(3-tert-butoxycarbonylaminopentylamino)piperidine-1-carboxylic acid tert-butyl ester. 163 mg of expected product are thus obtained after an additional step using a decarboxylation reaction following procedure 2 of example 8.

MH+=542; Melting point=264-267° C. (Isopropyl ether)

1H NMR (DMSO): 1.53 (d, 2); 1.91 (qui, 4); 2.02 (qd, 2); 2.83 (sxt, 2); 2.95 (q, 2); 3.17 to 3.28 (unresolved complex, 4); 4.00 (m, 1); 6.52 (d, 1); 7.26 (t, 2); 7.66 (dd, 2); 7.78 to 7.85 (unresolved complex, 4); 7.99 (bs, 3); 8.10 (d, 1); 8.84 to 8.99 (unresolved complex, 2); 10.61 to 11.09 (unresolved complex, 2).

### Example 152

### 4-({2-[(4-Fluoro-3-methoxyphenyl)amino]-pyrimidin-4-yl}amino)-N-methyl-N-(1-methylpiperidin-4-yl)benzenesulfonamide

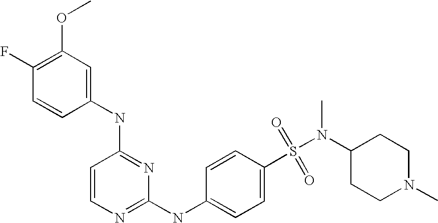

This product is prepared according to the scheme below which is therefore an illustration of the synthesis scheme 2 above.

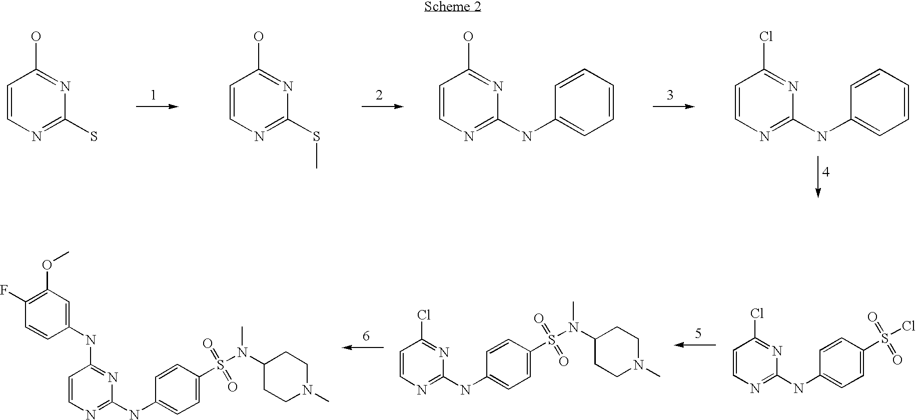

Stage 1: 2-(Methylthio)pyrimidin-4-ol: To a mixture containing 100 g of commercial 2-thiopyrimidin-4-ol, 60 g of sodium hydroxide in 800 ml of water, are added dropwise 38 ml of methyl iodide. The reaction medium is kept stirring at room temperature for 24 hours. The solution is acidified with 135 ml of acetic acid and left for 24 hours in a refrigerator. The white precipitate is filtered and washed several times with cold water. After drying, 60 g of expected compound are obtained.

Stage 2: 2-Anilinopyrimidin-4-ol: 39 g of 2-(methyl-thio)pyrimidin-4-ol are dissolved in 500 ml of DMF containing 30 ml of aniline. The reaction medium is kept stirring under reflux for 24 hours. After the usual treatment, 35.81 g of expected compound are obtained.

Stage 3: 4-Chloro-N-phenylpyrimidin-2-amine: A solution containing 15 g of 2-anilinopyrimidin-4-ol in 75 ml of POCl3 is heated at 110° C. for 2 hours. After evaporation of POCl3, the crude reaction product is poured into an ice-cold Na2CO3 solution. 16.3 g of expected product are obtained by filtration of the precipitate.

Stage 4: 2-[(2-Chloropyrimidin-4-yl)amino]benzenesulfonyl chloride: Following the procedure described in stage 3 of example 1, starting with 16.2 g of 4-chloro-N-phenylpyrimidin-2-amine, 7.6 g of expected product are obtained.

Stage 5: 4-[(4-Chloropyrimidin-2-yl)amino]-N-methyl-N-(1-methylpiperidin-4-yl)benzenesulfonamide: The procedure is carried out as in stage 4 of example 1, starting with 2 g of 4-[(4-Chloropyrimidin-2-yl)amino]benzenesulfonyl chloride and 0.96 ml of methyl(1-methylpiperidin-4-yl)amine. 1.88 g of expected product are thus obtained.

Stage 6: Following the procedure described in stage 2, starting with 1 g of 4-(2-chloropyrimidin-4-ylamino)-N-methyl-N-(1-methylpiperidin-4-yl)benzenesulfonamide and 0.428 g of 4-fluoro-3-methoxyphenylamine, 400 mg of expected product are obtained.

MH+=501.2

### Example 153

### 4-({4-[(4-Fluorophenyl)amino]pyrimidin-2-yl}amino)-N-(1-methylpiperidin-4-yl)-N-{2-[(2R)-pyrrol-idin-2-yl]ethyl}benzenesulfonamide

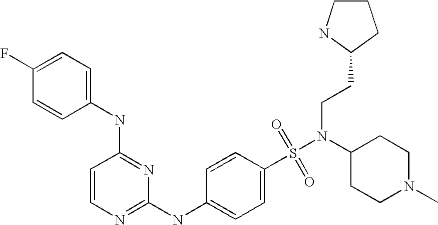

Stage 1: 2-[2-(1-Methylpiperidin-4-ylamino)ethyl]-pyrrolidine-1-carboxylic acid tert-butyl ester: As in example A, starting with 1 g of 1-methylpiperidin-4-one in the presence of 1.2 g of 2-(2-aminoethyl)pyrrolidine-1-carboxylic acid tert-butyl ester, 1.3 g of expected product are thus obtained ([α] (20,589).=−15.7° (C=0.18, MeOH)).

Stage 2: The procedure is carried out as in stage 4 of example 1, starting with 600 mg of 4-[4-(4-fluoro-phenylamino)pyrimidin-2-ylamino]benzenesulfonyl chloride hydrochloride and 497 mg of 2-[2-(1-methylpiperidin-4-ylamino)ethyl]pyrrolidine-1-carboxylic acid tert-butyl ester, 490 mg of an intermediate are isolated. After an additional step using a decarboxylation reaction following procedure 2 of example 8, 163 mg of expected product are obtained ([α] (20,589).=−13.9° (C=0.10, MeOH)).

MH+=554.2; Melting point=85° C. (Isopropyl ether/dichloromethane)

### Example 154

### 4-({4-[(4-Fluorophenyl)amino]pyrimidin-2-yl}amino)-N-(1-methylpiperidin-4-yl)-N-[2-(tetrahydro-furan-2-ylmethyl)ethyl]benzenesulfonamide

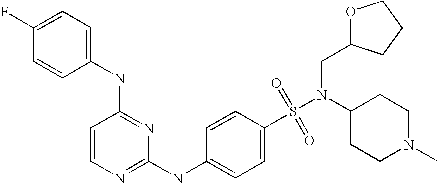

Stage 1: (1-Methylpiperidin-4-yl)(tetrahydrofuran-2-ylmethyl)amine: As in example A, starting with 1 g of 1-methylpiperidin-4-one in the presence of 1.08 ml of C-(tetrahydrofuran-2-yl)methylamine, 1.7 g of expected product are thus obtained.

Stage 2: The procedure is carried out as in stage 4 of example 1, starting with 600 mg of 4-[4-(4-fluoro-phenylamino)pyrimidin-2-ylamino]benzenesulfonyl chloride hydrochloride and 317 mg of (1-methylpiperidin-4-yl)(tetrahydrofuran-2-ylmethyl)amine, 300 mg of expected compound are obtained in the form of a racemic mixture.

MH+=541.2; Melting point=189° C. (Isopropyl ether/dichloromethane)

### Example 155

### N-(1,1-Dioxidotetrahydro-2H-thiopyran-4-yl)-4-({2-[(4-fluorophenyl)amino]pyrimidin-4-yl}amino)-N-methylbenzenesulfonamide

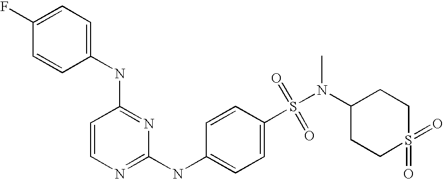

Stage 1: Benzyl(1,1-dioxidotetrahydro-2H-thiopyran-4-yl)methylamine: As in example A, starting with 2 g of benzyl(1,1-dioxohexahydro-1λ⁶-thiopyran-4-yl)amine in the presence of 1.3 ml of a 37% solution of formaldehyde, 2.1 g of expected product are thus obtained.

Stage 2: (1,1-Dioxidotetrahydro-2H-thiopyran-4-yl)-methylamine: 2.1 g of the compound described in stage 1 undergoes a hydrogenolysis reaction according to the process described in stage 4 of example 55 in order to give 1.5 g of expected product.

Stage 3: The procedure is carried out as in stage 4 of example 1, starting with 1.2 g of 4-[4-(4-fluoro-phenylamino)pyrimidin-2-ylamino]benzenesulfonyl chloride hydrochloride and 774 mg of (1,1-dioxidotetrahydro-2H-thiopyran-4-yl)methylamine, 140 mg of expected compound are obtained.

MH+=506.1; Melting point=251.7° C. (Isopropyl ether/dichloromethane)

### Example 156

### N-(1-Cyclopropylpiperidin-4-yl)-4-{[4-(4-fluoro-3-methoxyphenyl)pyrimidin-2-yl]amino}-N-(2-pyrrolidin-1-ylethyl)benzenesulfonamide

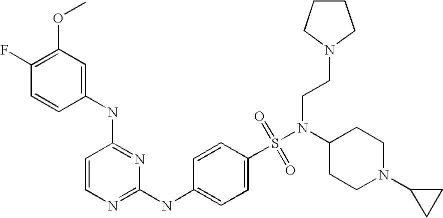

Stage 1: tert-Butyl 4-[({4-[(4-chloropyrimidin-2-yl)-amino]phenyl}sulfonyl)(2-pyrrolidin-1-ylethyl)amino]-piperidine-1-carboxylate: The procedure is carried out as in stage 4 of example 1, starting with 4 g of 4-[(2-chloropyrimidin-4-yl)amino]benzenesulfonyl chloride in stage 4 of example 152 and 3.92 g of 4-(2-pyrrolidin-1-ylethylamino)piperidine-1-carboxylic acid tert-butyl ester obtained in stage 1 of example 89. 6.67 g of expected compound are isolated.

Stage 2: 4-({4-[(4-Fluoro-3-methoxyphenyl)amino]-pyrimidin-2-yl}amino)-N-piperidin-4-yl-N-(2-pyrrolidin-1-ylethyl)benzenesulfonamide: Following the procedure described in stage 2 of example 152, starting with 1 g of tert-butyl 4-[({4-[(4-chloropyrimidin-2-yl)amino]-phenyl}sulfonyl)(2-pyrrolidin-1-ylethyl)amino]piperidine-1-carboxylate obtained in stage 1 and 300 mg of 4-fluoro-3-methoxyphenylamine, 822 mg of an intermediate are isolated. After an additional step using a decarboxylation reaction according to procedure 2 of example 8, 720 mg of expected product are obtained.

Stage 3: The procedure is carried out starting with 1 g of 4-({4-[(4-fluoro-3-methoxyphenyl)amino]pyrimidin-2-yl}amino)-N-piperidin-4-yl-N-(2-pyrrolidin-1-ylethyl)-benzenesulfonamide obtained in stage 2 and 920 mg of (1-ethoxycyclopropoxy)trimethylsilane. 21 mg of expected product are thus obtained.

MH+=610.1; Melting point=139° C. (Isopropyl ether/dichloromethane)

1H NMR (DMSO): 0.23 (bs, 2); 0.38 (m, 2); 1.34 (d, 2); 1.43-1.62 (unresolved complex, 4); 1.78 (bs, 4); 2.15 (t, 2); 2.90 (d, 2); 2.62-3.45 (unresolved complex, 6); 3.55 (t, 1); 3.81 (s, 3); 6.31 (d, 1); 7.16 (dd, 1); 7.20 (t, 1); 7.26 (m, 1); 7.36 (dd, 1); 7.68 (d, 2); 7.94 (d, 2); 8.09 (d, 1); 9.49 (bs, 1); 9.68 (bs, 1).

### Example 157

### 4-({4-[(4-Fluorophenyl)amino]pyrimidin-2-yl}amino)-N-methyl-N-(4-cis{1-[(1-methyl-1H-pyrrol-3-yl)methyl]amino}cyclohexyl)benzenesulfonamide

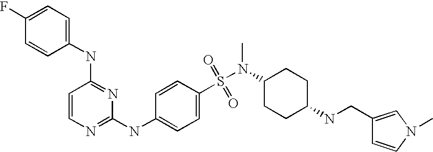

Following the procedure described in example 65, starting with 2 g of the compound of example 47 (60/40 trans and cis mixture) and 410 mg of methylpyrrole-3-carboxaldehyde makes it possible to have a mixture of two compounds. During the chromatography, both fractions are obtained including 310 mg of the expected cis isomer (least polar compound).

MH+=564.1

### Example 158

### 4-({4-[(4-Fluorophenyl)amino]pyrimidin-2-yl}amino)-N-methyl-N-(4-trans{1-[(1-methyl-1H-pyrrol-3-yl)methyl]amino}cyclohexyl)benzenesulfonamide

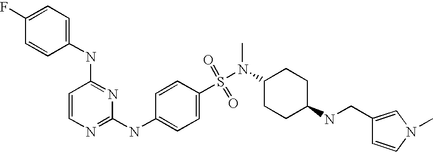

During the chromatography step of example 157, both fractions are obtained including 290 mg of the trans isomer.

MH+=564.1

### Example 159

### 4-({4-[(4-Fluorophenyl)amino]pyrimidin-2-yl}amino)-N-methyl-N-(8-methyl-8azabicyclo[3.2.1]oct-3-yl)benzenesulfonamide

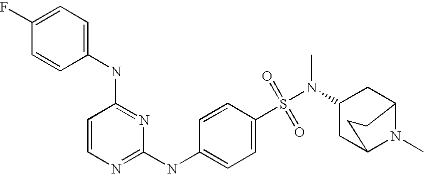

Stage 1:1-Methyl-(8-methyl-8-azabicyclo[3.2.1]oct-3-yl)amine: As in example A, starting with 3 g of 8-methyl-8-azabicyclo[3.2.1]octa-3-one in the presence of 21.55 ml of a 2N solution of methylamine in THF, 1.2 g of expected product are thus obtained.

Stage 2: The procedure is carried out as in stage 4 of example 1, starting with 600 mg of 4-[4-(4-fluoro-phenylamino)pyrimidin-2-ylamino]benzenesulfonyl chloride hydrochloride and 270 mg of 1-methyl-(8-methyl-8-azabicyclo[3.2.1]oct-3-yl)amine. 70 mg of expected compound are thus obtained.

MH+=497.2; Melting point=184.5° C. (Isopropyl ether/dichloromethane)

### Example 160

### 4-({2-[(3-Methyl-4-fluorophenyl)amino]-pyrimidin-2-yl}amino)-N-{1-[(1-methyl-1H-pyrrol-3-yl)-methyl]piperidin-4-yl}benzenesulfonamide

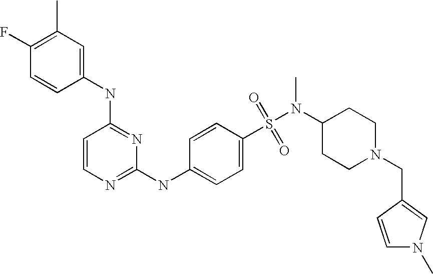

As in example A, starting with 270 mg of regenerated 4-({4-[(4-fluoro-3-methylphenyl)amino]pyrimidin-2-yl}-amino)-N-methyl-N-piperidin-4-ylbenzenesulfonamide (example 106) and 70 mg of methylpyrrole-3-carbox-aldehyde, 111 mg of expected product are obtained.

MH+=564.2

1H NMR (DMSO): 1.14 (m, 2); 1.76 (m, 2); 2.18 (s, 3); 2.59 (s, 3); 2.73 (m, 2); 3.13 (s, 2); 3.47 (s, 3); 3.53 (m, 1); 5.78 (m, 1); 6.22 (d, 1); 6.46 (m, 1); 6.51 (t, 1); 7.06 (t, 1); 7.0 (m, 1); 7.47-7.63 (d unresolved complex, 3); 7.88 (d, 2); 8.02 (d, 1); 9.37 (s, 1); 9.63 (s, 1).

### Example 161

### 4-({4-[(4-Fluorophenyl)amino]pyrimidin-2-yl}amino)-N-(1-methylpiperidin-4-yl)-N-[(1-methyl-1H-pyrrol-2-yl)methyl]benzenesulfonamide

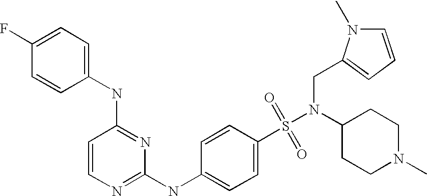

Stage 1: (1-Methylpiperidin-4-yl)(1-methyl-1H-pyrrol-2-ylmethyl)amine: As in example A, starting with 3 g of 1-methylpiperidin-4-one in the presence of 21.16 g of C-(1-methyl-1H-pyrrol-2-yl)methylamine, 2 g of expected product are thus obtained.

Stage 2: The procedure is carried out as in stage 4 of example 1 starting with 600 mg of 4-[4-(4-fluoro-phenylamino)pyrimidin-2-ylamino]benzenesulfonyl chloride hydrochloride and 330 mg of (1-methylpiperidin-4-yl)(1-methyl-1H-pyrrol-2-ylmethyl)amine. 80 mg of expected compound are thus obtained.

MH+=550.2; Melting point=129° C. (Isopropyl ether/dichloromethane)

### Example 162

### 4-[4-(3,4,5-Trifluorophenylamino)pyrimidin-2-ylamino]-N-methyl-N-(1-methylpiperidin-4-yl)benzenesulfonamide

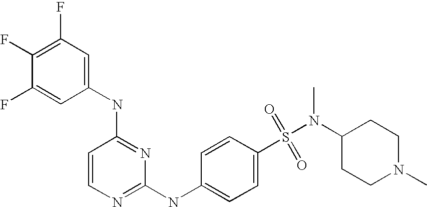

Following the procedure described in stage 6 of example 152, starting with 2 g of 4-(2-chloropyrimidin-4-yl-amino)-N-methyl-N-(1-methylpiperidin-4-yl)benzene-sulfonamide and 0.950 g of 3,4,5-trifluorophenylamine, 800 mg of expected product are obtained.

MH+=507.2; Melting point=226.9° C. (Isopropyl ether/dichloromethane)

### Example 163

### 4-({4-[(3,4-Difluorophenyl)amino]pyrimidin-2-yl}amino)-N-methyl-N-{1-[2-(methyl-sulfonyl)ethyl]piperidin-4-yl}benzenesulfonamide

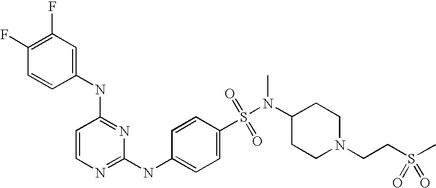

Following the procedure described in example 129, starting with 800 mg of 4-({4-[(3,4-difluorophenyl)-amino]pyrimidin-2-yl}amino)-N-methyl-N-piperidin-4-yl-benzenesulfonamide hydrochloride obtained in example 106 and 230 mg of methyl vinyl sulfone, 540 mg of desired compound are obtained, which compound crystallizes from an isopropyl ether-dichloromethane mixture.

MH+=581.2; Melting point=204° C. (Isopropyl ether/dichloromethane)

### Example 164

### 4-({4-[(3,4-Difluorophenyl)amino]pyrimidin-2-yl}amino)-N-methyl-N-{1-[(1-methyl-1H-pyrrol-2-yl)methyl]piperidin-4-yl)benzenesulfonamide

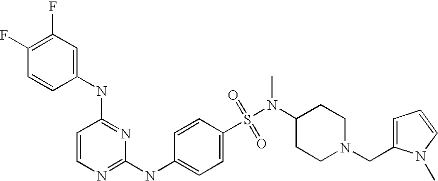

As in example A starting with 340 mg of regenerated 4-({4-[(3,4-difluorophenyl)amino]pyrimidin-2-yl}amino)-N-methyl-N-piperidin-4-ylbenzenesulfonamide (example 106) and 90 mg of 1-methyl-1H-pyrrole-3-carbaldehyde, 216 mg of expected product are obtained.

MH+=568.2; Melting point=149-152° C. (Isopropyl ether/dichloromethane)

### Example 165

### 4-({4-[(3,4-Difluorophenyl)amino]pyrimidin-2-yl}amino)-N-methyl-N-(1-{[1-(1,2,3)thiadiazol-4-yl]methyl}piperidin-4-yl)benzenesulfonamide

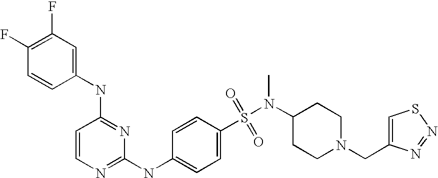

As in example A, starting with 350 mg of regenerated 4-({4-[(3,4-difluorophenyl)amino]pyrimidin-2-yl}amino)-N-methyl-N-piperidin-4-ylbenzenesulfonamide (example 106) and 100 mg of 1,2,3-thiadiazole-4-carbaldehyde, 271 mg of expected product are obtained.

MH+=573.1; Melting point=172-173° C. (Isopropyl ether/dichloromethane)

### Example 166

### 4-({4-[(3,4-Difluorophenyl)amino]pyrimidin-2-yl}amino)-N-methyl-N-{1-[(5-methylisoxazol-3-yl)methyl]piperidin-4-yl}benzenesulfonamide

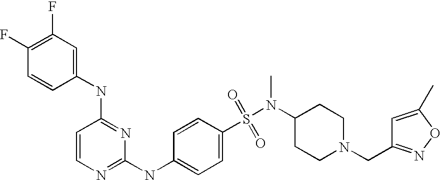

As in example A, starting with 350 mg of regenerated 4-({4-[(3,4-difluorophenyl)amino]pyrimidin-2-yl}amino)-N-methyl-N-piperidin-4-ylbenzenesulfonamide (example 106) and 90 mg of 5-methyl-3-isoxazole carbaldehyde, 244 mg of expected product are obtained.

MH+=570.0; Melting point=204-206° C. (Isopropyl ether/dichloromethane)

### Example 167

### 4-[4-(3,4-Difluorophenylamino)pyrimidin-2-ylamino]-N-[1-(2-hydroxy-2-methylpropyl)piperidin-4-yl]-N-methylbenzenesulfonamide

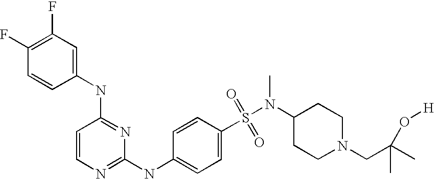

The procedure is carried out as in example 49, starting with 200 mg of 4-[4-(3,4-difluorophenylamino)pyrimidin-2-ylamino]-N-methyl-N-piperidin-4-ylbenzenesulfonamide (example 106) which are reacted with 0.05 ml of 1,2-epoxy-2-methylpropane in a microwave reactor (power: 200 W, temperature: 140° C.).

140 mg of expected product are thus obtained.

MH+=547.1; Melting point=234-235° C. (Isopropyl ether/dichloromethane)

### Example 168

### 4-({4-[(3-Methyl-4-fluorophenyl)amino]-pyrimidin-2-yl}amino)-N-(1-methylpiperidin-4-yl)-N-[(furan-2-yl)methyl]benzenesulfonamide

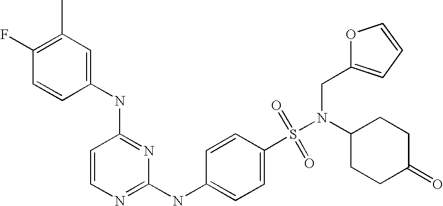

Stage 1: (1,4-Dioxaspiro[4.5]dec-8-yl)furan-2-ylmethyl-amine: As in example A, starting with 3 g of 1,4-dioxa-spiro[4.5]decan-8-one and 2.23 g of furan-2-methylamine, 4.3 g of expected product are obtained.

Stage 2: N-1,4-Dioxaspiro[4.5]dec-8-yl-4-({4-[(4-fluorophenyl)amino]pyrimidin-2-yl}amino)-N-(furan-2-yl-methyl)benzenesulfonamide: The procedure is carried out as in stage 4 of example 1, starting with 4 g of 4-({4-[(3-methyl-4-fluorophenyl)amino]pyrimidin-2-yl}amino)-benzenesulfonyl chloride hydrochloride and 2.85 g of (1,4-dioxaspiro[4.5]dec-8-yl)furan-2-ylmethylamine. 500 mg of expected product are thus obtained.

Stage 3: Following the procedure described in example 117, starting with 750 mg of N-1,4-dioxaspiro[4.5]dec-8-yl-4-({4-[(4-fluorophenyl)amino]pyrimidin-2-yl}amino)-N-(furan-2-ylmethyl)benzenesulfonamide. 670 mg of expected product are thus obtained.

MH+=550.2; Melting point=184° C. (Isopropyl ether/dichloromethane)

### Example 169

### 4-({4-[(3-Methyl-4-fluorophenyl)amino]-pyrimidin-2-yl}amino)-N-(1-methylpiperidin-4-yl)-N-[(tetrahydrofuran-2-yl)methyl]benzenesulfonamide

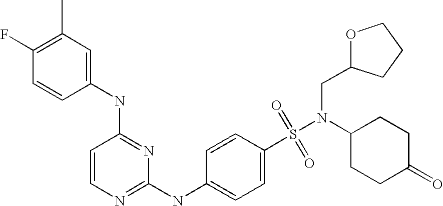

Stage 1: (1,4-Dioxaspiro[4.5]dec-8-yl)(tetrahydrofuran-2-ylmethyl)amine: As in example A, starting with 3 g of 1,4-dioxaspiro[4.5]decan-8-one and 2.33 g of tetrahydrofuran-2-methylamine, 4.2 g of expected product are obtained.

Stage 2: N-1,4-Dioxaspiro[4.5]dec-8-yl-4-({4-[(4-fluorophenyl)amino]pyrimidin-2-yl}amino)-N-(tetrahydro-furan-2-yl-methyl)benzenesulfonamide: The procedure is carried out as in stage 4 of example 1, starting with 3 g of 4-({4-[(3-methyl-4-fluorophenyl)amino]pyrimidin-2-yl}amino)benzenesulfonyl chloride hydrochloride and 2.21 mg of (1,4-dioxaspiro[4.5]dec-8-yl)(tetrahydrofuran-2-ylmethyl)amine. 1.8 g of expected product are thus obtained.

Stage 3: Following the procedure described in example 117, starting with 1.5 g of N-1,4-dioxaspiro-[4.5]dec-8-yl-4-({4-[(4-fluorophenyl)amino]pyrimidin-2-yl}amino)-N-(tetrahydrofuran-2-ylmethyl)benzenesulfon-amide. 1.1 g of expected product are thus obtained.

MH+=554.0; Melting point=189° C. (Isopropyl ether/dichloromethane)

### Example 170

### Pharmaceutical Composition

Tablets having the following formula were prepared:

| | |
|---|---|
| Product of example 3 | 0.2 g |
| Excipient for a finished tablet at | 1 g |
| (details of the excipient: lactose, talc, | |
| starch, magnesium stearate). | |

### Example 171

### Pharmaceutical Composition

Tablets having the following formula were prepared:

| | |
|---|---|
| Product of example 12 | 0.2 g |
| Excipient for a finished tablet at | 1 g |
| (details of the excipient: lactose, talc, | |
| starch, magnesium stearate). | |

### Example 172

### Pharmaceutical Composition

Tablets having the following formula were prepared:

| | |
|---|---|
| Product of example 25 | 0.2 g |
| Excipient for a finished tablet at | 1 g |
| (details of the excipient: lactose, talc, | |
| starch, magnesium stearate). | |

Examples 3, 12 and 25 are taken as examples in the pharmaceutical preparations which constitute examples 29 to 31 above, it being possible for this pharmaceutical preparation to be made differently as indicated above and if desired with other products in the examples in the present application.

### Pharmacological Part:

### Protocols for Biochemical Assays on IKK.

### I) Evaluation of the Compounds on IKK1 and IKK2:

The compounds are tested for the inhibition of IKK1 and IKK2 using a kinase test on a flash plate support. The test compounds are dissolved in 10 mM in DMSO and then diluted in kinase buffer (50 mM Tris, pH 7.4 containing 0.1 mM EGTA, 0.1 mM sodium orthovanadate and 0.1% p-mercaptoethanol).

Three-fold serial dilutions are made from this solution. 10 μl of each dilution are added to the wells of a 96-well plate in duplicate. 10 μl of kinase buffer are added to the control wells which will serve as 0% inhibition and 10 μl of 0.5 mM EDTA are added to the control wells (100% inhibition). 10 μl of the mixture IKK1 or IKK2 (0.1 μg/well), substrate peptide 25-55 IKB-biotinylated and BSA (5 μg) are added to each well. In order to initiate the kinase reaction, 10 μl of the mixture of 10 mM magnesium acetate, 1 μM cold ATP and 0.1 μCi 33P-ATP are added to each well for a final volume of 30 μl. The reaction is incubated at 30° C. for 90 min and then stopped by the addition of 40 μl of 0.5 mM EDTA. After stirring, 50 μl are transferred to a streptavidin-coated flash plate.

30 min later, the wells are washed twice with a solution of 50 mM Tris-EDTA pH 7.5 and the radioactivity is determined on a microbeta counter.

The compounds of the invention tested in this assay show an IC50 of less than 10 μM, which shows that they can be used for their therapeutic activity.

### II) Evaluation of the Compounds on the Viability and the Proliferation of Tumor Cells:

The compounds according to the invention were the subject of pharmacological trials which make it possible to determine their anticancer activity.

The compounds of formula (I) according to the present invention were tested in vitro on a panel of tumor lines of human origin obtained from:
breast cancer: MDA-MB231 (American Type culture collection, Rockville, Md., USA, ATCC-HTB26), MDA-A1 or MDA-ADR (termed multi-drug resistant MDR line, and described by E. Collumb et al., in Cytometry, 12 (1):15-25, 1991), and MCF7 (ATCC-HTB22),
prostate cancer: DU145 (ATCC-HTB81) and PC3 (ATCC-CRL1435),
colon cancer: HCT116 (ATCC-CCL247) and HCT15 (ATCC-CCL225),
lung cancer: H460 (described by Carmichael in Cancer Research 47 (4):936-942, 1987 and provided by National Cancer Institute, Frederick Cancer Research and Development Center, Frederick, Md., USA),
glioblastoma (SF268 described by Westphal in Biochemical & Biophysical Research Communications 132 (1): 284-289, 1985 and provided by National Cancer Institute, Frederick Cancer Research and Development Center, Frederick, Md., USA),
leukemia (CMLT1 described by Kuriyama et al. in Blood, 74: 1989, 1381-1387, by Soda et al. in British Journal of Haematology, 59: 1985, 671-679 and by Drexler, in Leukemia Research, 18: 1994, 919-927 and provided by the company DSMZ, Mascheroder Weg 1b, 38124 Brunswick, Germany).

The cell proliferation and viability were determined in a test using 3-(4,5-dimethylthiazol-2-yl)-5-(3-carboxy-methoxyphenyl)-2-(4-sulfophenyl)-2H-tetrazolium (MTS) according to Fujishita T. et al., Oncology, 2003, 64 (4), 399-406. In this test, the mitochondrial capacity of living cells to convert MTS to a colored compound after 72 hours of incubation of a compound of formula (I) according to the invention is measured. The concentrations of compound according to the invention which lead to a 50% loss of cell proliferation and viability (IC50) are less than 10 μM, depending on the tumor line and the test compound.

Thus, according to the present invention, it is apparent that the compounds of formula (I) cause a loss of tumor cell proliferation and viability with an IC50 of less than 10 μM.

What is claimed is:

1) A compound of formula (I): 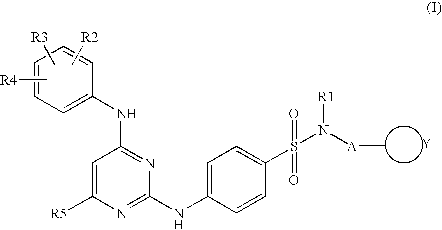
in which:
R2, R3 and R4, which are identical or different, are such that one represents a halogen atom and the other two, which are identical or different, represent a hydrogen atom or a halogen atom or an alkyl radical or an alkoxy radical;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9, the alkyl radicals which R1 represents being further optionally substituted with a 5-membered saturated or unsaturated heterocyclic radical attached by a carbon atom and optionally substituted with one or more radicals chosen from halogen atoms and alkyl or alkoxy radicals;
A represents a single bond or a radical —CH2-CO—NR6-, and R6, which is identical to or different from R1, is chosen from the values of R1;
the ring containing Y (or ring (Y)) consists of 4 to 8 members and is saturated or partially saturated, with Y representing an oxygen atom O, a sulfur atom S optionally oxidized with one or two oxygen atoms or a radical chosen from N—R7, C═O or its dioxolane as protecting group for the carbonyl functional group, CF2, CH—OR8 or CH—NR8R9;
it being understood that the ring containing Y (or ring (Y)), when Y represents NR7, may contain a carbon bridge consisting of 1 to 3 carbons,
R7 represents a hydrogen atom, a cycloalkyl radical or an alkyl, CH2-alkenyl or CH2-alkynyl radical, all optionally substituted with a naphthyl radical or with one or more radicals, which are identical or different, chosen from halogen, hydroxyl, alkoxy, phenyl and heteroaryl radicals, the alkyl radicals which R7 represents being further optionally substituted with a phosphonate radical, with an alkylthio radical optionally oxidized to a sulfone or with an optionally substituted heterocycloalkyl radical,
R8 represents a hydrogen atom or a alkyl, cycloalkyl or heterocycloalkyl radical which are themselves optionally substituted with one or more radicals chosen from halogen, hydroxyl, alkoxy, NH2, NHalkyl or N(alkyl)2 radicals, the alkyl radicals which R8 represents being further optionally substituted with an alkylthio radical, with an optionally substituted phenyl radical or with an optionally substituted saturated or unsaturated heterocyclic radical,
NR8R9 is such that either R8 and R9, which are identical or different, are chosen from the values of R8, or R8 and R9 form with the nitrogen atom to which they are attached, a cyclic amine which may optionally contain one or two other heteroatoms chosen from O, S, N or NR10, the cyclic amine thus formed being itself optionally substituted with one or more alkyl radicals;
all the above heterocyclic, heterocycloalkyl and heteroaryl radicals consisting of 4 to 10 members (unless specified otherwise) and containing 1 to 3 heteroatoms chosen, where appropriate, from O, S, N and NR10;
all the above naphthyl, phenyl, heterocyclic, heterocycloalkyl and heteroaryl radicals being themselves optionally substituted with one or more radicals, which are identical or different, chosen from halogen atoms and hydroxyl, alkoxy, alkyl, hydroxyalkyl, alkoxyalkyl, CN, CF3, NH2, NHalk and N(alk)2 radicals;
R10 represents a hydrogen atom or an alkyl radical;
said compound being in all the possible racemic, enantiomeric and diastereoisomeric isomer forms;
or an addition salt with an inorganic or organic acid of said compound.

2) The compound according to claim 1, in which R2, R3, R4, R5, A and ring (Y) have the meanings indicated in claim 1;
R1 represents a hydrogen atom or an alkyl radical containing from 1 to 5 carbon atoms, which is linear or branched, or alternatively R1 represents an alkyl radical containing from 1 to 5 carbon atoms and substituted with a 5-membered saturated or unsaturated heterocycle which is itself optionally substituted as indicated in claim 1;
said compound being in all the possible racemic, enantiomeric and diastereoisomeric isomer forms;
or an addition salt with an inorganic and organic acid of said compound.

3) The compound according to claim 1, in which R2, R3, R4, R5 and A have the meanings indicated in claim 1;
R1 represents a hydrogen atom or an optionally substituted linear or branched alkyl radical containing from 1 to 4 carbon atoms;
ring (Y) is such that Y represents NR7 with R7 representing a linear or branched alkyl radical containing from 1 to 6 carbon atoms substituted with a radical chosen from hydroxyl, CF3, phosphonate, sulfone, phenyl and saturated or unsaturated monocyclic or bicyclic heterocyclic radicals, the phenyl and heterocyclic radicals being themselves optionally substituted as indicated in claim 1;
said compound being in all the possible racemic, enantiomeric and diastereoisomeric isomer forms;
or an addition salt with an inorganic and organic acid of said compound.

4) A compound of formula (I): 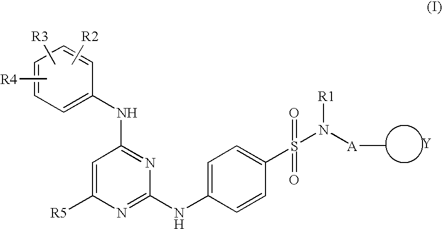
in which:
R2, R3 and R4, which are identical or different, are such that one represents a halogen atom and the other two, which are identical or different, represent a hydrogen atom or a halogen atom or an alkyl radical or an alkoxy radical;
R5 represents a hydrogen atom or a halogen atom;
R1 represents a linear or branched alkyl radical containing from 1 to 4 carbon atoms;
A represents a single bond or a radical —CH2-CO—NR6-, and R6, which is identical to or different from R1, is chosen from the values of R1;
ring (Y) is such that Y represents CH—NR8R9 in which R8 represents a hydrogen atom or CH3 and R9 represents a linear or branched alkyl radical containing from 1 to 6 carbon atoms substituted with a radical chosen from hydroxyl, CF3, phosphonate, sulfone, phenyl and saturated or unsaturated monocyclic or bicyclic heterocyclic radicals, the phenyl and heterocyclic radicals being themselves optionally substituted;
all the above heterocyclic radicals consisting of 4 to 10 members (unless specified) and containing 1 to 3 heteroatoms chosen, where appropriate, from O, S, N and NR10;
all the above phenyl and heterocyclic radicals being themselves optionally substituted with one or more radicals, which are identical or different, chosen from halogen atoms and hydroxyl, alkoxy, alkyl, hydroxyalkyl, alkoxyalkyl, CN, CF3, NH2, NHalk and N(alk)2 radicals;
R10 represents a hydrogen atom or an alkyl radical;
said compound being in all the possible racemic, enantiomeric and diastereoisomeric isomer forms;
or an addition salt with an inorganic and organic acid of said compound.

5) The compound according to claim 1, in which:
R2, R3 and R4, which are identical or different, are such that one represents a halogen atom and the other two, which are identical or different, represent a hydrogen atom or a halogen atom or an alkyl radical;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9;
A represents a single bond or a radical —CH2-CO—NR6-, and R6, which is identical to or different from R1, is chosen from the values of R1;
the ring containing Y consisting of 4 to 8 members and is saturated or partially saturated, with Y representing an oxygen atom O, a sulfur atom S optionally oxidized with one or two oxygen atoms or a radical chosen from N—R7, C═O, CF2, CH—OR8 or CH—NR8R9;
R7 represents a hydrogen atom or an alkyl, CH2-alkenyl or CH2-alkynyl radical, all optionally substituted with a naphthyl radical or with one or more radicals which are identical or different, chosen from halogen atoms and hydroxyl, phenyl and heteroaryl radicals, all these naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals;
the heteroaryl radicals consisting of 5 to 10 members and containing 1 to 3 heteroatoms chosen from O, S, N and NR10;
R8 represents a hydrogen atom or alkyl, cycloalkyl or heterocycloalkyl radicals which are themselves optionally substituted with one or more radicals chosen from hydroxyl, alkoxy, NH2, Nalkyl or N(alkyl)2 radicals;
NR8R9 is such that either R8 and R9, which are identical or different, are chosen from the values of R8 or R8 and R9 form with the nitrogen atom to which they are attached a cyclic amine which may optionally contain one or two other heteroatoms chosen from O, S, N or NR10;
R10 represents a hydrogen atom or an alkyl radical;
said compound being in all the possible racemic, enantiomeric and diastereoisomeric isomer forms;
or an addition salt with an inorganic and organic acid of said compound.

6) The compound according to claim 1, in which:
R2, R3 and R4, which are identical or different, are such that one represents a halogen atom and the other two, which are identical or different, represent a hydrogen atom or a halogen atom;
R5 represents a hydrogen atom or a halogen atom;
R1 represents a hydrogen atom, a cycloalkyl radical or an alkyl, alkenyl or alkynyl radical, all optionally substituted with one or more radicals, which are identical or different, chosen from halogen atoms, OR8 and NR8R9;
A represents a single bond or a radical —CH2-CO—NR6-, and R6, which is identical to or different from R1, is chosen from the values of R1;
the ring containing Y consists of 4 to 8 members and is saturated or partially saturated, with Y representing an oxygen atom O, a sulfur atom S optionally oxidized with one or two oxygen atoms, or a radical chosen from N—R7, C═O, CF2, CH—OR8 and CH—NR8R9;
R7 represents a hydrogen atom or an alkyl, CH2-alkenyl or CH2-alkynyl radical, all optionally substituted with a naphthyl radical or with one or more radicals, which are identical or different, chosen from halogen atoms and phenyl and heteroaryl radicals, the naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk and N(alk)2 radicals;
the heteroaryl radicals consisting of 5 to 10 members and containing 1 to 3 heteroatoms chosen from O, S, N and NR10;
R8 represents a hydrogen atom or alkyl, cycloalkyl or heterocycloalkyl radical, which are themselves optionally substituted with one or more radicals chosen from hydroxyl, alkoxy, NH2, Nalkyl or N(alkyl)2 radicals;
NR8R9 is such that either R8 and R9, which are identical or different, are chosen from the values of R8, or R8 and R9 form with the nitrogen atom to which they are attached a cyclic amine which may optionally contain one or two other heteroatoms chosen from O, S, N and NR10;
R10 represents a hydrogen atom or an alkyl radical;
said compound being in all the possible racemic, enantiomeric and diastereoisomeric isomer forms;
or an addition salt with an inorganic and organic acid of said compound.

7) The compound according to claim 1, in which:
R2, R3 and R4, which are identical or different, are such that one represents a fluorine or chlorine atom and the other two, which are identical or different, represent a hydrogen atom or a fluorine or chlorine atom;
R5 represents a hydrogen atom or a fluorine or chlorine atom;
R1 represents a hydrogen atom, a cycloalkyl radical or an alkyl radical optionally substituted with one or more radicals, which are identical or different, chosen from a fluorine atom, OR8 and NR8R9;
A represents a single bond or a radical —CH2-CO—NR6-, and R6 represents a hydrogen atom or a linear or branched alkyl radical containing 1 to 4 carbon atoms;
the ring containing Y consists of 4 to 7 members and is saturated or partially saturated, with Y representing an oxygen atom O, a sulfur atom S optionally oxidized with one or two oxygen atoms or a radical chosen from N—R7, C═O, CF2, CH—OR8 or CH—NR8R9;
R7 represents a hydrogen atom or an alkyl radical optionally substituted with one or more radicals, which are identical or different, chosen from halogen atoms and phenyl and heteroaryl radicals, the phenyl and heteroaryl radicals themselves being optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals;
the heteroaryl radicals consisting of 5 to 7 members and containing 1 to 3 heteroatoms chosen from O, S, N and NR10;
R8 represents a hydrogen atom, a linear or branched alkyl radical containing 1 to 4 carbon atoms or a cycloalkyl radical containing 3 to 6 members, the alkyl and cycloalkyl radicals are themselves optionally substituted with a hydroxyl radical;
NR8R9 is such that either R8 and R9, which are identical or different, are chosen from the values of R8, or R8 and R9 form with the nitrogen atom to which they are attached a cyclic amine chosen from pyrrolyl, piperidyl, morpholinyl, pyrrolidinyl, azetidinyl and piperazinyl radicals, optionally substituted on its second atom with an alkyl radical;
said compound being in all the possible racemic, enantiomeric and diastereoisomeric isomer forms;
or an addition salt with an inorganic and organic acid of said compound.

8) The compound according to claim 1, in which:
R2, R3 and R4, which are identical or different, are such that one represents a fluorine atom and the other two, which are identical or different, represent a hydrogen atom or a fluorine or chlorine atom;
R5 represents a hydrogen atom or a chlorine atom;
R1 represents a hydrogen atom, a cycloalkyl radical or an alkyl radical optionally substituted with one or more radicals chosen from a fluorine atom and a hydroxyl, amino, methylamino, dimethylamino, piperidinyl, morpholinyl, azetidinyl and piperazinyl radical;
A represents a single bond or a radical —CH2-CO—NR6-, and R6 represents a hydrogen atom or an alkyl radical containing 1 or 2 carbon atoms;
the ring containing Y consists of 4 to 7 members, being saturated, with Y representing an oxygen atom O, a sulfur atom S optionally oxidized with one or two oxygen atoms, or a radical chosen from N—R7, CH—NH2, CH—NHalk and CH—N(alk)2;
R7 represents a hydrogen atom or an alkyl radical optionally substituted with a phenyl, pyridyl, thienyl, thiazolyl, pyrazinyl, furyl or imidazolyl radical which are themselves optionally substituted with one or more radicals chosen from halogen atoms and hydroxyl, methoxy, methyl, hydroxymethyl, methoxymethyl, trifluoromethyl, amino, methylamino and dimethylamino radicals;
said compound being in all the possible racemic, enantiomeric and diastereoisomeric isomer forms;
or an addition salt with an inorganic and organic acid of said compound.

9) The compound according to claim 1, in which:
R2, R3 and R4, which are identical or different, are such that one represents a fluorine atom and the other two represent, one a hydrogen atom and the other a fluorine or chlorine atom or a methyl radical;
R5 represents a hydrogen atom or a chlorine atom;
R1 represents a hydrogen atom; a cyclopropyl radical; a methyl radical; or an ethyl, propyl or butyl radical optionally substituted with a fluorine atom or a hydroxyl radical or an amino, alkylamino, dialkylamino or pyrrolidinyl radical;
A represents a single bond, —CH2-CO—NH— or —CH2-CO—NCH3- and the ring containing Y is chosen from a cyclohexyl radical, itself substituted with amino; tetrahydropyran; dioxidothienyl; and a pyrrolidinyl, piperidinyl and azepinyl radical optionally substituted on their nitrogen atom with a methyl, propyl, isopropyl, isobutyl, isopentyl or ethyl radical, themselves optionally substituted with one or more radicals chosen from halogen atoms and a hydroxyl radical, a phenyl radical which is itself optionally substituted with one or more halogen atoms, a quinolyl radical, a pyridyl radical which is optionally oxidized on its nitrogen atom, a thienyl radical, a thiazolyl radical, a pyrazinyl radical, a furyl radical and an imidazolyl radical which is itself optionally substituted with alkyl;
said compound being in all the possible racemic, enantiomeric and diastereoisomeric isomer forms;
or an addition salt with an inorganic and organic acid of said compound.

10) The compound according to claim 1, in which:
R2, R3 and R4, which are identical or different, are such that one represents a fluorine atom and the other two represent, one a hydrogen atom and the other a fluorine or chlorine atom or a methyl radical;
R5 represents a hydrogen atom;
R1 represents a methyl radical; or an ethyl radical, optionally substituted with an amino, alkylamino, dialkylamino or pyrrolidinyl radical;
A represents a single bond and the ring containing Y represents a cyclohexyl radical which is itself substituted with amino or Y is a piperidinyl radical optionally substituted on its nitrogen atom with a methyl, propyl, isopropyl, isobutyl, isopentyl or ethyl radical, which are themselves optionally substituted with one or more halogen atoms or a radical chosen from hydroxyl; phenyl which is itself substituted with halogen; quinolyl; pyridyl optionally oxidized on its nitrogen atom; furyl; and imidazolyl which is itself optionally substituted with alkyl;
said compound being in all the possible racemic, enantiomeric and diastereoisomeric isomer forms;
or an addition salt with an inorganic and organic acid of said compound.

11) The compound according to claim 1, in which:
R2, R3 and R4, which are identical or different, are such that one represents a fluorine atom and the other two represent, one a hydrogen atom and the other a fluorine or chlorine atom;
R5 represents a hydrogen atom or a chlorine atom;
R1 represents a hydrogen atom; a cyclopropyl radical; a methyl radical; or an ethyl, propyl or butyl radical optionally substituted with a fluorine atom or a hydroxyl radical or a dialkylamino radical;
A represents a single bond, —CH2-CO—NH— or —CH2-CO—NCH3- and the ring containing Y is chosen from tetrahydropyran and dioxidothienyl radicals and pyrrolidinyl, piperidinyl and azepinyl radicals optionally substituted on their nitrogen atom with a methyl or ethyl radical, which are themselves optionally substituted with a phenyl, pyridyl, thienyl, thiazolyl, pyrazinyl, furyl or imidazolyl radical;
said compound being in all the possible racemic, enantiomeric and diastereoisomeric isomer forms;
or an addition salt with an inorganic and organic acid of said compound.

12) The compound according to claim 1 selected from the group consisting of:
2-{4-[4-(4-fluorophenylamino)pyrimidin-2-ylamino]-benzenesulfonylamino}-N-(tetrahydropyran-4-yl)-acetamide;
4-[4-(4-fluorophenylamino)pyrimidin-2-ylamino]-N-methyl-N-(1-pyridin-2-ylmethylpiperidin-4-yl)benzenesulfonamide;
N-(2-dimethylaminoethyl)-4-[4-(4-fluorophenylamino)-pyrimidin-2-ylamino]-N-(1-methylpiperidin-4-yl)benzene-sulfonamide;
4-[4-(4-fluorophenylamino)pyrimidin-2-ylamino]-N-(2-hydroxyethyl)-N-(1-methylpiperidin-4-yl)benzene-sulfonamide;
4-[4-(4-fluorophenylamino)pyrimidin-2-ylamino]-N-methyl-N-(1-pyridyl-3-ylmethylpiperidin-4-yl)benzenesulfonamide;
4-[4-(4-fluorophenylamino)pyrimidin-2-ylamino]-N-piperidin-4-yl-N-(2-pyrrolidin-1-ylethyl)benzenesulfon-amide hydrochloride; and
N-(2-aminoethyl)-4-[4-(3-chloro-4-fluorophenylamino)-pyrimidin-2-ylamino]-N-piperidin-4-ylbenzenesulfonamide hydrochloride;
said compound being in all the possible racemic, enantiomeric and diastereoisomeric isomer forms;
or an addition salt with an inorganic or organic acid of said compound.

13) The compound according to claim 1 selected from the group consisting of:
2-{4-[4-(4-fluorophenylamino)pyrimidin-2-ylamino]-benzenesulfonylamino}-N-(tetrahydropyran-4-yl)acetamide;
4-[4-(4-fluorophenylamino)pyrimidin-2-ylamino]-N-methyl-N-(1-pyridin-2-ylmethylpiperidin-4-yl)benzenesulfonamide;
N-(2-dimethylaminoethyl)-4-[4-(4-fluorophenylamino)-pyrimidin-2-ylamino]-N-(1-methylpiperidin-4-yl)benzene-sulfonamide;
4-[4-(4-fluorophenylamino)pyrimidin-2-ylamino]-N-(2-hydroxyethyl)-N-(1-methylpiperidin-4-yl)benzenesulfon-amide; and
4-[4-(4-fluorophenylamino)pyrimidin-2-ylamino]-N-methyl-N-(1-pyridyl-3-ylmethylpiperidin-4-yl)benzenesulfonamide;
said compound in all the possible racemic, enantiomeric and diastereoisomeric isomer forms;
or an addition salt with an inorganic or organic acid of said compound.

14) A process for preparing the compound of formula (I) according to claim 1, said process comprising reacting a compound of formula (II): 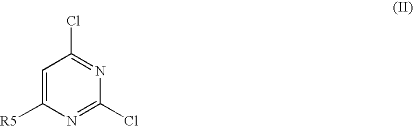
in which R5 has the meaning indicated in claim 1,
with a compound of formula (III): 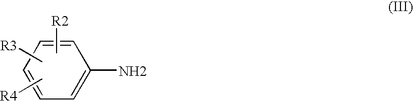
in which R2, R3 and R4 have the meanings indicated in claim 1,
in order to obtain a compound of formula (IV), 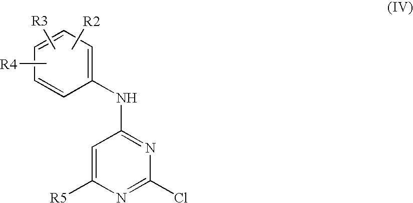
in which R2, R3, R4 and R5 have the meanings indicated above,
which compound of formula (IV) is reacted with the aniline of formula (V): 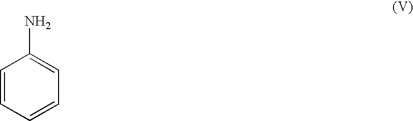
in order to obtain a compound of formula (VI): 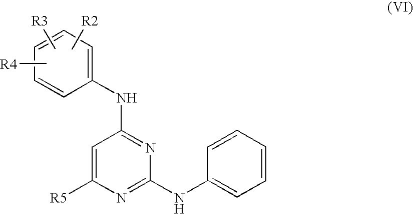
in which R2, R3, R4 and R5 have the meanings indicated above,
which compound of formula (VI) is reacted with chlorosulfonic acid SO2(OH)Cl in order to obtain the corresponding compound of formula (VII): 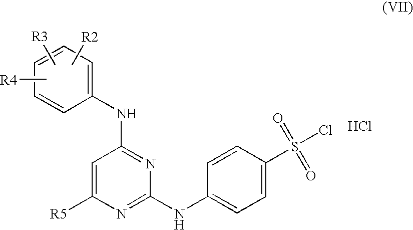
in which R2, R3, R4 and R5 have the meanings indicated above, which compound of formula (VII) is reacted with an amine or formula (VIII): 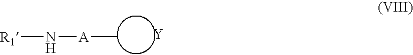
in which A and Y have the meanings indicated in claim 1 and R₁′ has the meaning indicated in claim 1 for R1, in which the reactive functional groups which may be present are optionally protected with protecting groups,
in order to obtain a compound of formula (I₁): 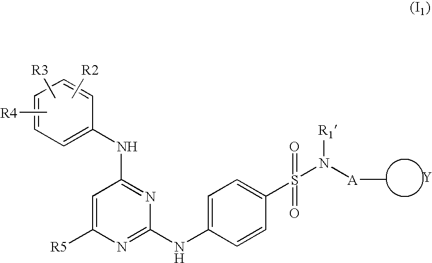
in which R₁′, R2, R3, R4, R5, A, and Y have the meanings indicated above,
which compound of formula (I₁) may be a compound of formula (I) and which, in order to obtain a compound of formula (I) or another compound of formula (I), may be subjected, if desired and if necessary, to one or more of the following conversion reactions in any order:
a) a reaction for oxidation of an alkylthio group to the corresponding sulfoxide or sulfone,
b) a reaction for conversion of an alkoxy functional group to a hydroxyl functional group, or alternatively of a hydroxyl functional group to an alkoxy functional group,
c) a reaction for oxidation of an alcohol functional group to an aldehyde or ketone functional group,
d) a reaction for removal of the protecting groups which the protected reactive functional groups may carry,
e) a reaction for salification with an inorganic or organic acid in order to obtain the corresponding salt,
f) a reaction for resolution of the racemic forms to resolved compounds,
said compound of formula (I) thus obtained being in all the possible racemic, enantiomeric and diastereoisomeric isomer forms.

15) A process for preparing a compound of formula (I) as defined in claim 1, in which Y represents the radical NR7 as defined in claim 1, R7 represents CH2-RZ and RZ represents an alkyl, alkenyl or alkynyl radical, all optionally substituted with a naphthyl radical or with one or more radicals, which are identical or different, chosen from halogen atoms and phenyl and heteroaryl radicals, the naphthyl, phenyl and heteroaryl radicals being themselves optionally substituted with one or more radicals, which are identical or different, chosen from halogen atoms and hydroxyl, alkoxy, alkyl, hydroxyalkyl, alkoxyalkyl, CF3, NH2, NHalk or N(alk)2 radicals, which process comprises subjecting the compound of formula (A): 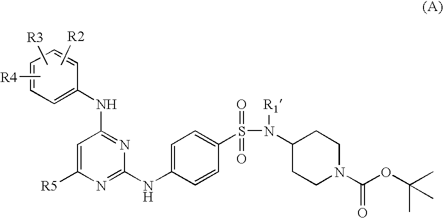
in which R2, R3, R4 and R5 have the meanings indicated in claim 1, and R₁′ has the meaning indicated in claim 1 for R1, in which the reactive functional groups which may be present are optionally protected with protecting groups, to a reaction for deprotection of the carbamate functional group in order to obtain a compound of formula (IX): 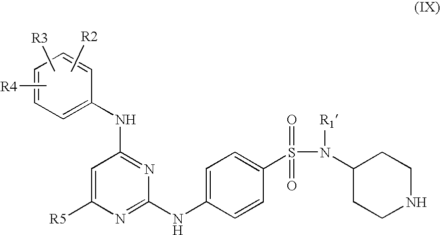
in which R₁′, R2, R3, R4 and R5 have the meanings indicated above,
which compound of formula (IX) is subjected to reductive amination conditions
in the presence of the aldehyde of formula (X):
RZ′-CHO (X)
in which RZ′ has the meaning indicated above for RZ, in which the reactive functional groups which may be present are optionally protected with protecting groups, in order to obtain a compound of formula (I₂): 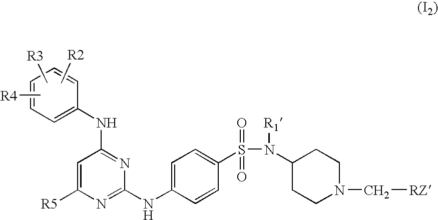
in which R₁′, R2, R3, R4, R5 and RZ′ have the meanings indicated above,
which compound of formula (I₂) may be a compound of formula (I) and which, in order to obtain a compound of formula (I) or another compound of formula (I), may be subjected, if desired and if necessary, in any order, to one or more conversion reactions a) to f) as defined above,
said compound of formula (I₂) thus obtained being in all the possible racemic, enantiomeric and diastereoisomeric isomer forms.

16) A pharmaceutical composition comprising, as active ingredient, at least one compound of formula (I) according to claim 1 or a pharmaceutically acceptable salt of said compound or a prodrug of said compound, and a pharmaceutically acceptable carrier.

17) A pharmaceutical composition comprising, as active ingredient, at least one compound of formula (I) according to claim 12 or a pharmaceutically acceptable salt of said compound or a prodrug of said compound, and a pharmaceutically acceptable carrier.

18) A method for treating or preventing a disease or disorder by inhibiting the protein kinase IKK, comprising administering to a patient in need thereof a therapeutically effective amount of a compound of formula (I) according to claim 1 or a pharmaceutically acceptable salt thereof.

19) The method according to claim 18, wherein the protein kinase IKK is in a mammal.

20) The method according to claim 18 wherein the disease or disorder is chosen from the group consisting of inflammatory diseases, diabetes and cancers.

21) The method according to claim 18 wherein the disease or disorder is an inflammatory disease.

22) The method according to claim 18 wherein the disease or disorder is diabetes.

23) The method for treating a disease or disorder by inhibiting the protein kinase IKK according to claim 18, wherein the disease or disorder is cancer.

24) The method according to claim 23 wherein the compound is administered in combination with one or more anticancer active ingredients.

25) The method according to claim 23 wherein the cancer is resistant to cytotoxic agents.

26) The method for treating a disease or disorder by inhibiting the protein kinase IKK according to claim 18, wherein the disease or disorder is a solid or liquid tumor.
